(19) United States
(12) Patent Application Publication
(10) Pub. No.: US 2024/0043883 A1
(43) Pub. Date: Feb. 8, 2024

(54) Synthesis Of 3-Hydroxypropionic Acid Via Hydration Of Acetylenecarboxylic Acid

(71) Applicant: Board of Trustees of Michigan State University, East Lansing, MI (US)

(72) Inventors: Karen F. DRATHS, Okemos, MI (US); James H. GEIGER, Webberville, MI (US); Amaya Nethmini Sirinimal MATHES HEWAGE, Lansing, MI (US); Hadi NAYEBI GAVGANI, Lansing, MI (US)

(21) Appl. No.: 18/485,646

(22) Filed: Oct. 12, 2023

Related U.S. Application Data
(63) Continuation of application No. PCT/US2022/025756, filed on Apr. 21, 2022.
(60) Provisional application No. 63/178,821, filed on Apr. 23, 2021.

(51) Int. Cl. C12P 7/42; C12N 9/04; C12N 9/90

(52) U.S. Cl. CPC C12P 7/42; C12N 9/0006; C12N 9/90; C12Y 503/02006

(57) ABSTRACT

An in vitro and/or in vivo method of producing malonic semialdehyde (MSA) or an anion or salt thereof and/or 3-hydroxypropionic acid (3-HP) or an anion or salt thereof is provided herein. The method may comprise two steps: (1) hydrating acetylenecarboxylic acid (ACA) or an anion or salt thereof by reacting the ACA or an anion or salt thereof with an ACA-hydrating enzyme to form a reaction product comprising malonic semialdehyde (MSA) or an anion or salt thereof; and (2) reacting the reaction product comprising MSA or an anion or salt thereof with one or more oxidoreductases in an oxidation-reduction (redox) reaction to produce 3-HP or an anion or salt thereof A pair of oxidoreductases may additionally recycle a cofactor, such as NADPH or NADH. Recombinant microbes and compositions are also provided herein which may include ACA-hydrating enzymes or variants thereof, and/or one or more oxidoreductase enzymes.

### CROSS REFERENCE TO RELATED APPLICATIONS

The present application is a continuation under 35 U.S.C. § 120 of International Patent Application No. PCT/US2022/025756 designating the United States and filed on 21 Apr. 2022, which claims the benefit under 35 U.S.C. § 119(e) of U.S. Provisional Patent Application No. 63/178,821 filed on 23 Apr. 2021. The entire contents of each application recited above is hereby incorporated by reference.

### REFERENCE TO A SEQUENCE LISTING

This application contains references to amino acid sequences and/or nucleic acid sequences which have been submitted concurrently herewith as the sequence listing XML file entitled “ST26 SL Conversion_25_Sept_2023”, file size 143 KiloBytes (KB), created on 25 Sep. 2023. The aforementioned sequence listing is hereby incorporated by reference in its entirety pursuant to 37 C.F.R. § 1.52(e)(5).

### FIELD

The present disclosure relates to the transformation of acetylenecarboxylic acid (ACA) into 3-hydroxypropionic acid (3-HP).

### BACKGROUND

This section provides background information related to the present disclosure which is not necessarily prior art. This section also provides a general summary of the disclosure, and is not a comprehensive disclosure of its full scope or all of its features.

3-HP is an achiral, 3-carbon β-hydroxycarboxylic acid. A 2004 U.S. Department of Energy report identified 3-HP among 15 chemicals whose synthesis from biomass or synthesis gas would benefit the economics of the biorefinery. Like petroleum refineries, economic success for integrated biorefineries will require production of relatively high value, low volume chemicals to offset losses incurred from production of low value, high volume transportations fuels. Inclusion of 3-HP on both the original list and a revisited list of chemical targets is based on existing 3-HP market demands, the potential for new applications, and its conversion into additional chemicals with existing markets. The most noteworthy characteristic of 3-HP is its versatility for transformation into various chemicals with established applications, including production of polymers, fibers, resins, adhesives, paints and coatings.

### SUMMARY

This disclosure provides an in vitro method for producing malonic semialdehyde (MSA) or an anion or salt thereof and for producing 3-HP or an anion or salt thereof that uses an ACA-hydrating enzyme, one or more oxidoreductase enzymes, and a cofactor. One step includes reacting ACA or an anion or salt thereof with an ACA-hydrating enzyme to produce a reaction product comprising MSA or an anion or salt thereof. Another step includes reacting MSA or an anion or salt thereof with one or more oxidoreductases in a redox reaction to produce 3-HP or an anion or salt thereof. The redox reaction may include a pair of oxidoreductases to cycle a cofactor, such as NADPH or NADH. Alternatively, the redox reaction may only include one oxidoreductase enzyme and may not cycle a cofactor. Also disclosed herein are compositions comprising an ACA-hydrating enzyme and/or one or more oxidoreductases. The composition may produce MSA or an anion or salt thereof and/or 3-HP or an anion or salt thereof.

Additionally, this disclosure provides an in vivo method for producing MSA or an anion or salt thereof and for producing 3-HP (for example, see FIG. 1) or an anion or salt thereof that uses an ACA-hydrating enzyme, one or more oxidoreductase enzymes, and a cofactor. One step includes reacting ACA or an anion or salt thereof with an ACA-hydrating enzyme to produce a reaction product comprising MSA or an anion or salt thereof. Another step includes reacting MSA or an anion or salt thereof with one or more oxidoreductases in a redox reaction to produce 3-HP or an anion or salt thereof. The redox reaction may include a pair of oxidoreductases to cycle a cofactor, such as NADPH or NADH. Alternatively, the redox reaction may only include one oxidoreductase enzyme. In this case, one or more enzymes native to the production host cell may regenerate or recycle the cofactor.

Also described herein are recombinant microbes comprising an ACA-hydrating enzyme and/or one or more oxidoreductases. The recombinant microbe may be a recombinant bacteria, a recombinant yeast, or a recombinant algae. The recombinant microbe may produce MSA or an anion or salt thereof and/or 3-HP or an anion or salt thereof.

Additionally described herein are variant enzymes capable of hydrating ACA. The variant ACA-hydrating enzymes may be substantially free of decarboxylase activity and/or have hydratase-only activity. The variant ACA-hydrating enzymes may generate more MSA compared to a control ACA-hydrating enzyme. In some embodiments, the variant ACA-hydrating enzyme may be Cg10062 with an E114N mutation. Also provided herein are vectors and recombinant cells encoding the variant ACA-hydrating enzyme.

### BRIEF DESCRIPTION OF THE DRAWINGS

FIG. 1 is a schematic representation of in vitro synthesis of 3-hydroxypropionic acid (3-HP) from ACA achieved using three enzymes: Cg10062 (E114N) (SEQ ID NO: 62), a variant of Cg10062 from C. glutamicum; a 3-hydroxy acid dehydrogenase (YdfG) (SEQ ID NO: 75) from E. coli and a previously engineered phosphite dehydrogenase, PTDH (SEQ ID NO: 73) from P. stutzeri.

FIG. 2 is a schematic representation of ACA and acetylenedicarboxylic acid (ADCA) synthesis via acetylene from CH₄ and CO₂.

FIG. 3 is a graph representing the conversion of 100 mM ACA into 3-HP with co-factor recycling over a period of 30 hours.

FIG. 4A-4C depicts ¹H NMR of 3-HP synthesis from 100 mM ACA with FIG. 4A) 0.1 FIG. 4B) 0.01 and FIG. 4C) 0.001 eq NADP(H).

FIG. 5 is a graph representing the conversion of 500 mM ACA to 3-HP with co-factor recycling over a period of 61 h.

FIG. 6A-6C depicts ¹H NMR of 3-HP synthesis from 500 mM ACA with FIG. 6A) 0.1, FIG. 6B) 0.01 and FIG. 6C) 0.001 eq NADP(H).

FIG. 7 is a graph representing pH dependence of Cg10062 (E114N) (SEQ ID NO: 62).

FIG. 8 is a graph representing pH dependence of YdfG (SEQ ID NO: 75).

FIG. 9 is a graph representing pH dependence of PTDH (SEQ ID NO: 73).

FIG. 10A-10B depicts FIG. 10A) ¹H NMR of 3-HP formed from ACA in vivo in uninduced (top) and FIG. 10B) IPTG-induced (bottom) LB cultures.

FIG. 11A-11B depicts FIG. 11A) ¹H NMR of 3-HP formed from ACA in vivo in uninduced (top) and FIG. 11B) IPTG-induced (bottom) M9 cultures.

FIG. 12A-12C represents nucleotide sequences of FIG. 12A) Cg10062 (wild-type) (SEQ ID NO: 41) (NCBI-MZ369159) FIG. 12B) Cg10062 (E114N) (SEQ ID NO: 44) and FIG. 12C) MSAD (SEQ ID NO: 56) (NCBI-MZ369160), codon-optimized for expression in E. coli. Highlighted nucleotides at the end of the sequences encode a TEV protease recognition sequence followed by a His₆-tag for affinity purification, connected by 6 nucleotides.

FIG. 13 is a schematic representation of the coupled enzyme assay used to measure hydratase and hydratase/decarboxylase activity of Cg10062 (wild-type) (SEQ ID NO: 59) and variants thereof. The asterisk indicates acetaldehyde produced by mutants with hydratase/decarboxylase activity.

FIG. 14A-14E includes graphs depicting Michaelis-Menten kinetics of FIG. 14A) Cg10062 (SEQ ID NO: 59), FIG. 14B) Cg10062 (E114D) (SEQ ID NO: 61), FIG. 14C) Cg10062 (E114Q) (SEQ ID NO: 60), FIG. 14D) Cg10062 (E114D-Y103F) (SEQ ID NO: 71) and FIG. 14E) Cg10062 (E114N) (SEQ ID NO: 62).

FIG. 15A-15B depicts ¹H NMR spectra of Cg10062 (SEQ ID NO: 59)-catalyzed hydration of ACA at FIG. 15A) 0 h and FIG. 15B) 1 h.

FIG. 16A-16B depicts ¹H NMR spectra of Cg10062 (E114N) (SEQ ID NO: 62)-catalyzed hydration of ACA at FIG. 16A) 0 h and FIG. 16B) 1 h.

FIG. 17A-17B depicts ¹H NMR spectra of Cg10062 (E114Q) (SEQ ID NO: hydration of ACA at FIG. 17A) 0 h and FIG. 17B) 1 h.

FIG. 18A-18B depicts ¹H NMR spectra of Cg10062 (E114D) (SEQ ID NO: 61)-catalyzed hydration of ACA at FIG. 18A) 0 h and FIG. 18B) 1 h.

FIG. 19 is a schematic representation of the hydration of ACA by Cg10062 (E114N) (SEQ ID NO: 62) coupled to the reduction of malonic semialdehyde (MSA) to 3-HP by YdfG (SEQ ID NO: 75). The activity is followed by the loss of absorbance at 340 nm due to NADPH oxidation.

FIG. 20 is a graph depicting Michaelis Menten kinetics of YdfG (SEQ ID NO: 75).

FIG. 21A-21B depicts ¹H NMR spectra of FIG. 21A) authentic 3-HP and FIG. 21B) 3-HP produced by YdfG (SEQ ID NO: 75).

FIG. 22 is a schematic representation of PTDH (SEQ ID NO: 73) activity that was monitored following the reduction of NADP⁺ at 340 nm.

FIG. 23 is a graph depicting Michaelis Menten Kinetics of PTDH (SEQ ID NO: 73).

FIG. 24 is a schematic representation of in vitro synthesis of 3-HP from ACA achieved using three enzymes: Cg10062 (E114N) (SEQ ID NO: 62), a variant of Cg10062 from C. glutamicum; 3-hydroxyisobutyrate dehydrogenase (MmsB) (SEQ ID NO:76) from P. putida KT2440; and soluble hydrogenase (SH) (described in para. 100) from C. necator.

FIG. 25 is a graph depicting the synthesis of 3-HP from ACA using Cg10062 (E114N) (SEQ ID NO: 62), MmsB (SEQ ID NO:76), and SH (described in para. 100).

FIG. 26A-26B depicts ¹H NMR spectra of 3-HP synthesis from 12.5 mM ACA with FIG. 26A) 0.2 and FIG. 26B) 0.02 eq NAD(H).

FIG. 27 is a graph depicting pH dependence of MmsB (SEQ ID NO:76).

FIG. 28 is a schematic representation of the hydration of ACA by Cg10062 (E114N) (SEQ ID NO:62) coupled to the reduction of MSA to 3-HP by MmsB (SEQ ID NO:76).

FIG. 29 is a graph depicting Michaelis Menten kinetics of MmsB (SEQ ID NO:76).

FIG. 30 is a schematic representation of monitored SH (described in para. 100) activity following the reduction of NAD⁺ at 365 nm.

### DETAILED DESCRIPTION

### I. Definitions

The following definitions refer to the various terms used above and throughout the disclosure.

As used herein, singular articles such as “a” and “an” and “the” and similar referents in the context of describing the elements are to be construed to cover both the singular and the plural, unless otherwise indicated herein or clearly contradicted by context.

As used herein, “about” is understood by persons of ordinary skill in the art and may vary to some extent depending upon the context in which it is used. If there are uses of the term which are not clear to persons of ordinary skill in the art given the context in which the term “about” is used, “about” will mean up to plus or minus 10% of the particular term.

As will be understood by one skilled in the art, for any and all purposes, all ranges disclosed herein also encompass any and all possible subranges and combinations of subranges thereof. Furthermore, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Unless defined otherwise, technical and scientific terms used herein have the same meaning as commonly understood by a person of ordinary skill in the art. In particular, this disclosure utilizes routine techniques in the field of recombinant genetics, organic chemistry, and biochemistry.

Sequence Accession numbers throughout this description were obtained from databases provided by the NCBI (National Center for Biotechnology Information) maintained by the National Institutes of Health, U.S.A. (which are identified herein as “NCBI Accession Numbers” or alternatively as “GenBank Accession Numbers” or alternatively a simply “Accession Numbers”), and from the UniProt Knowledgebase (UniProtKB) and Swiss-Prot databases provided by the Swiss Institute of Bioinformatics (which are identified herein as “UniProtKB Accession Numbers”).

The term “enzyme classification (EC) number” refers to a number that denotes a specific polypeptide sequence or enzyme. EC numbers classify enzymes according to the reaction they catalyze. EC numbers are established by the nomenclature committee of the international union of biochemistry and molecular biology (IUBMB), a description of which is available on the IUBMB enzyme nomenclature website on the world wide web.

As used herein, the terms “isolated” and “purified,” with respect to products (such as MSA and 3-HP), refer to products that are separated from cellular components, cell culture media, or chemical or synthetic precursors.

As used herein, the terms “polypeptide” and “protein” are used interchangeably to refer to a polymer of amino acid residues that is typically 12 or more amino acids in length. Polypeptides less than 12 amino acids in length are referred to herein as “peptides.” The terms apply to amino acid polymers in which one or more amino acid residue is an artificial chemical mimetic of a corresponding naturally occurring amino acid, as well as to naturally occurring amino acid polymers and non-naturally occurring amino acid polymers. The term “recombinant polypeptide” refers to a polypeptide that is produced by recombinant techniques, wherein generally DNA or RNA encoding the expressed protein is inserted into a suitable expression vector that is in turn used to transform a host cell to produce the polypeptide. In some exemplary embodiments, DNA or RNA encoding an expressed peptide, polypeptide, or protein is inserted into the host chromosome via homologous recombination or other means well known in the art, and is so used to transform a host cell to produce the peptide or polypeptide. Similarly, the terms “recombinant polynucleotide” or “recombinant nucleic acid” or “recombinant DNA” are produced by recombinant techniques that are known to those of skill in the art (see e.g., methods described in Sambrook et al. (Sambrook et al., Molecular Cloning—A Laboratory Manual, Cold Spring Harbor Press 4th Edition (Cold Spring Harbor, N.Y. 2012) and/or Current Protocols in Molecular Biology (Volumes 1-3, John Wiley & Sons, Inc. (1994-1998) and Supplements 1-115 (1987-2016).).

When referring to two nucleotide or polypeptide sequences, the “percentage of sequence identity” between the two sequences is determined by comparing the two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The “percentage of sequence identity” is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Thus, the expression “percent identity,” or equivalently “percent sequence identity,” “homology, or “homologous” in the context of two or more nucleic acid sequences or peptides or polypeptides, refers to two or more sequences or subsequences that are the same or have a specified percentage of nucleotides or amino acids that are the same (e.g., about 50% identity, preferably 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured e.g., using a BLAST or BLAST 2.0 sequence comparison algorithm with default parameters (see e.g., Altschul et al. (1990) J. Mol. Biol. 215(3):403-410) and/or the NCBI web site at ncbi.nlm.nih.gov/BLAST/) or by manual alignment and visual inspection. Percent sequence identity between two nucleic acid or amino acid sequences also can be determined using e.g., the Needleman and Wunsch algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6 (Needleman and Wunsch (1970) J. Mol. Biol. 48:444-453). The percent sequence identity between two nucleotide sequences also can be determined using the GAP program in the GCG software package, using a NWSgapdna.CMP matrix and a gap weight of 40, 50, 60, 70, or 80 and a length weight of 1, 2, 3, 4, 5, or 6. One of ordinary skill in the art can perform initial sequence identity calculations and adjust the algorithm parameters accordingly. A set of parameters that may be used if a practitioner is uncertain about which parameters should be applied to determine if a molecule is within a sequence identity limitation of the claims, are a Blossum 62 scoring matrix with a gap penalty of 12, a gap extend penalty of 4, and a frameshift gap penalty of 5. Additional methods of sequence alignment are known in the biotechnology arts (see, e.g., Rosenberg (2005) BMC Bioinformatics 6:278; Altschul et al. (2005) FEBS J. 272(20):5101-5109).

Two or more nucleic acid or amino acid sequences are said to be “substantially identical,” when they are aligned and analyzed as discussed above and are found to share about 50% identity, preferably 55%, 60%, 65%, 70%, 75%, 80%, 85%, 90%, 91%, 92%, 93%, 94%, 95%, 96%, 97%, 98%, 99%, or higher identity over a specified region. Two nucleic acid sequences or polypeptide sequences are said to be “identical” if the sequence of nucleotides or amino acid residues, respectively, in the two sequences are the same when aligned for maximum correspondence as described above. This definition also refers to, or may be applied to, the complement of a test sequence. Identity is typically calculated over a region that is at least about 25 amino acids or nucleotides in length, or more preferably over a region that is 50-100 amino acids or nucleotides in length, or over the entire length of a given sequence.

The term “endogenous” as used herein refers to a substance e.g., a nucleic acid, protein, etc. that is produced from within a cell. Thus, an endogenous polynucleotide or polypeptide refers to a polynucleotide or polypeptide produced by the cell. In some exemplary embodiments an endogenous polypeptide or polynucleotide is encoded by the genome of the parental cell (or host cell). In other exemplary embodiments, an endogenous polypeptide or polynucleotide is encoded by an autonomously replicating plasmid carried by the parental cell (or host cell). In some exemplary embodiments, an endogenous gene is a gene that was present in the cell when the cell was originally isolated from nature i.e., the gene is native to the cell. In other exemplary embodiments, an “endogenous” gene has been altered through recombinant techniques e.g., by altering the relationship of control and/or coding sequences. Thus, a heterologous gene, in some exemplary embodiments, may be endogenous to a host cell. Additionally, a variant (i.e. mutant) polypeptide encoded by the heterologous gene and produced within the cell would be considered endogenous polypeptide.

In contrast, an “exogenous” polynucleotide or polypeptide, or other substance (e.g., ACA-hydrating enzyme derivative, small molecule compound, etc.) refers to a polynucleotide or polypeptide or other substance that is not encoded or produced by the cell and which is therefore added to a cell, a cell culture, or assay from outside of the cell. A variant (i.e., mutant) polypeptide added to the cell, cell culture, or assay is one example of an exogenous polypeptide.

As used herein the term “native” refers to the form of a nucleic acid, protein, polypeptide or a fragment thereof that is isolated from nature or a nucleic acid, protein, polypeptide or a fragment thereof that is in its natural state without intentionally introduced mutations in the structural sequence and/or without any engineered changes in expression such as e.g., changing a developmentally regulated gene to a constitutively expressed gene. As used herein, “native” also refers to “wildtype” or “wild-type,” in which the nucleic acid, protein, polypeptide, or a fragment thereof is present in both sequence, quantity, and relative quantity as typically found in the organism as naturally found.

The term “non-native” is used herein to refer to nucleic acid sequences, amino acid sequences, proteins and derivatives thereof, and/or small molecules that do not occur naturally in the host. Heterologous genes are considered “non-native.” A nucleic acid sequence or amino acid sequence that has been removed from a host cell, subjected to laboratory manipulation, and introduced or reintroduced into a host cell is considered “non-native.” Synthetic or partially synthetic genes introduced into a host cell are “non-native.” Non-native genes further include genes endogenous and/or native to the host microorganism but operably linked to one or more heterologous regulatory sequences that have been recombined into the host genome. A naturally occurring gene under the control of a heterologous regulatory sequence is considered “non-native.” In some embodiments, an organism comprising a non-native gene may be utilized as a control and/or reference for an organism having additional and/or different variations from wild-type organisms.

The term “gene” as used herein, refers to nucleic acid sequences e.g., DNA sequences, which encode either an RNA product or a protein product, as well as operably-linked nucleic acid sequences that affect expression of the RNA or protein product (e.g., expression control sequences such as e.g., promoters, enhancers, ribosome binding sites, translational control sequences, etc). The term “gene product” refers to either the RNA (e.g., tRNA, mRNA) and/or protein expressed from a particular gene.

The term “expression” or “expressed” as used herein in reference to a gene, refers to the production of one or more transcriptional and/or translational product(s) of a gene. In exemplary embodiments, the level of expression of a DNA molecule in a cell is determined on the basis of either the amount of corresponding mRNA that is present within the cell or the amount of protein encoded by that DNA produced by the cell. The term “expressed genes” refers to genes that are transcribed into messenger RNA (mRNA) and then translated into protein, as well as genes that are transcribed into other types of RNA, such as e.g., transfer RNA (tRNA), ribosomal RNA (rRNA), and regulatory RNA, which are not translated into protein.

The level of expression of a nucleic acid molecule in a cell or cell free system is influenced by “expression control sequences” or equivalently “regulatory sequences” or “regulatory elements.” Expression control sequences, regulatory sequences, or regulatory elements are known in the art and include, for example, promoters, enhancers, polyadenylation signals, transcription terminators, nucleotide sequences that affect RNA stability, internal ribosome entry sites (IRES), and the like, that provide for the expression of the polynucleotide sequence in a host cell. In exemplary embodiments, “expression control sequences” interact specifically with cellular proteins involved in transcription (see e.g., Maniatis et al., Science, 236: 1237-1245 (1987); Goeddel, Gene Expression Technology: Methods in Enzymology, Vol. 185, Academic Press, San Diego, Calif (1990)). In exemplary methods, an expression control sequence, regulatory sequence, or regulatory element is operably linked to a polynucleotide sequence. By “operably linked” is meant that a polynucleotide sequence and an expression control sequence(s) or regulatory element(s) are functionally connected so as to permit expression of the polynucleotide sequence when the appropriate molecules (e.g., transcriptional activator proteins) contact the expression control sequence(s). In exemplary embodiments, operably linked promoters are located upstream of the selected polynucleotide sequence in terms of the direction of transcription and translation. In some exemplary embodiments, operably linked enhancers may be located upstream, within, or downstream of the selected polynucleotide.

As used herein, the phrase “expression of said nucleotide sequence is modified relative to the wild-type nucleotide sequence,” refers to a change e.g., an increase or decrease in the level of expression of a native nucleotide sequence or a change e.g., an increase or decrease in the level of the expression of a heterologous or non-native polypeptide-encoding nucleotide sequence as compared to a control nucleotide sequence e.g., wild-type control. In some exemplary embodiments, the phrase “the expression of said nucleotide sequence is modified relative to the wild-type nucleotide sequence,” refers to a change in the pattern of expression of a nucleotide sequence as compared to a control pattern of expression e.g., constitutive expression as compared to developmentally timed expression.

A “control” sample (e.g., a control nucleotide sequence, a control polypeptide sequence, a control cell, etc., or value) refers to a sample that serves as a reference, usually a known reference, for comparison to a test sample. For example, in an exemplary embodiment, a test sample comprises a 3-HP composition made by a recombinant microbe that comprises a heterologous, genetically manipulated ACA-hydrating enzyme or variant thereof as disclosed herein, while the control sample comprises a 3-HP composition made by the corresponding or designated microbe that comprises a non-genetically manipulated ACA-hydrating enzyme. Additionally, a control cell or microorganism may be referred to as a corresponding wild-type or host cell. One of skill will recognize that controls may be designed for assessment of any number of parameters. Furthermore, one of skill in the art will understand which controls are valuable in a given situation and will be able to analyze data based on comparisons to control values.

The term “overexpressed” or “up-regulated” as used herein, refers to a gene whose expression is elevated in comparison to a control level of expression. In exemplary embodiments, overexpression of a gene is caused by an elevated rate of transcription as compared to the native transcription rate for that gene. In other exemplary embodiments, overexpression is caused by an elevated rate of translation of the gene compared to the native translation rate for that gene. Methods of testing for overexpression are well known in the art, for example transcribed RNA levels may be assessed using rtPCR and protein levels may be assessed using SDS page gel analysis.

In other embodiments, the polypeptide, polynucleotide, or hydrocarbon having an altered level of expression is “attenuated” or has a “decreased level of expression” or is “down-regulated.” As used herein, these terms mean to express or cause to be expressed a polynucleotide, polypeptide, or hydrocarbon in a cell at a lesser concentration than is normally expressed in a corresponding control cell (e.g., wild-type cell) under the same conditions. In other words, the term “attenuate” means to weaken, reduce, or diminish. For example, a polypeptide can be attenuated by modifying the polypeptide to reduce its activity (e.g., by modifying a nucleotide sequence that encodes the polypeptide).

A polynucleotide or polypeptide can be attenuated using any method known in the art. For example, in some exemplary embodiments, the expression of a gene or polypeptide encoded by the gene is attenuated by mutating the regulatory polynucleotide sequences which control expression of the gene. In other exemplary embodiments, the expression of a gene or polypeptide encoded by the gene is attenuated by overexpressing a repressor protein, or by providing an exogenous regulatory element that activates a repressor protein. In still other exemplary embodiments, DNA- or RNA-based gene silencing methods are used to attenuate the expression of a gene or polynucleotide. In some embodiments, the expression of a gene or polypeptide is completely attenuated, e.g., by deleting all or a portion of the polynucleotide sequence of a gene.

The degree of overexpression or attenuation may be 1.5-fold or more, e.g., 2-fold or more, 3-fold or more, 5-fold or more, 10-fold or more, or 15-fold or more. Alternatively, or in addition, the degree of overexpression or attenuation may be 500-fold or less, e.g., 100-fold or less, 50-fold or less, 25-fold or less, or 20-fold or less. Thus, the degree of overexpression or attenuation may be bounded by any two of the above endpoints. For example, the degree of overexpression or attenuation may be 1.5-500-fold, 2-50-fold, 10-25-fold, or 15-20-fold.

As used herein, “substantially free” refers to a condition wherein the recombinant microbe comprises none or almost none of the component it is deemed to be “substantially free” of For example, the recombinant microbe would be substantially free of the component if it contained less than about 5 wt %, less than about 4 wt %, less than about 3 wt %, less than about 2 wt %, less than about 1 wt %, less than about 0.5 wt %, less than about 0.1 wt %, less than about 0.05 wt %, less than about 0.01 wt %, or about 0 wt % of the component normally found in the microbe. Alternatively, the term “substantially free” may refer to a low amount of the component in relation to another component within the recombinant microbe. For example, a recombinant E. coli is substantially free of acetaldehyde if the acetaldehyde comprises about 5 wt % or less of the total amount of components within the E coli. Alternatively, the recombinant E. coli would be considered substantially free of acetaldehyde if the acetaldehyde comprises less than about 4 wt %, less than about 3 wt %, less than about 2 wt %, less than about 1 wt %, less than about 0.5 wt %, less than about 0.1 wt %, less than about 0.05 wt %, less than about 0.01 wt %, or about 0 wt % of the total amount of components within the E coli.

As used herein, “modified activity” or an “altered level of activity” of a protein/polypeptide in a recombinant host cell refers to a difference in one or more characteristics in the activity the protein/polypeptide as compared to the characteristics of an appropriate control protein e.g., the corresponding parent protein or corresponding wild-type protein. Thus, in exemplary embodiments, a difference in activity of a protein having “modified activity” as compared to a corresponding control protein is determined by measuring the activity of the modified protein in a recombinant host cell and comparing that to a measure of the same activity of a corresponding control protein in an otherwise isogenic host cell. Modified activities may be the result of, for example, changes in the structure of the protein (e.g., changes to the primary structure, such as e.g., changes to the protein's nucleotide coding sequence that result in changes in substrate specificity, changes in observed kinetic parameters, changes in solubility, etc.); changes in protein stability (e.g., increased or decreased degradation of the protein) etc.

The term “heterologous” as used herein refers to a polypeptide or polynucleotide which is in a non-native state. Thus, a polynucleotide or a polypeptide is “heterologous” to a cell when the polynucleotide and/or the polypeptide and the cell are not found in the same relationship to each other in nature. Therefore, a polynucleotide or polypeptide sequence is “heterologous” to an organism or a second sequence if it originates from a different organism, different cell type, or different species, or, if from the same species, it is modified from its original form. Thus, in an exemplary embodiment, a polynucleotide or polypeptide is “heterologous” when it is not naturally present in a given organism. For example, a polynucleotide sequence that is native to cyanobacteria may be introduced into a host cell of E. coli (a proteobacterium) by recombinant methods, and the polynucleotide from cyanobacteria is then heterologous to the E. coli cell (i.e., the now recombinant E. coli cell). Alternatively, a polynucleotide or polypeptide would be considered “heterologous” if expression of the polynucleotide or polypeptide is different from the expression level native to that organism.

Similarly, a polynucleotide or polypeptide is heterologous when it is modified from its native form or from its relationship with other polynucleotide sequences or is present in a recombinant host cell in a non-native state. Thus, in an exemplary embodiment, a heterologous polynucleotide or polypeptide comprises two or more subsequences that are not found in the same relationship to each other in nature. For example, a promoter operably linked to a nucleotide coding sequence derived from a species different from that from which the promoter was derived. Alternatively, in another example, if a promoter is operably linked to a nucleotide coding sequence derived from a species that is the same as that from which the promoter was derived, then the operably-linked promoter and coding sequence are “heterologous” if the coding sequence is not naturally associated with the promoter (e.g. a constitutive promoter operably linked to a developmentally regulated coding sequence that is derived from the same species as the promoter). In other exemplary embodiments, a heterologous polynucleotide or polypeptide is modified relative to the wild-type sequence naturally present in the corresponding wild-type host cell, e.g., an intentional modification e.g., an intentional mutation in the sequence of a polynucleotide or polypeptide or a modification in the level of expression of the polynucleotide or polypeptide. Typically, a heterologous nucleic acid or polynucleotide is recombinantly produced.

The term “recombinant” as used herein, refers to a genetically modified polynucleotide, polypeptide, cell, tissue, or organism. When used with reference to a cell, the term “recombinant” indicates that the cell has been modified by the introduction of a heterologous nucleic acid or protein or has been modified by alteration of a native nucleic acid or protein, or that the cell is derived from a cell so modified and that the derived cell comprises the modification. Thus, for example, “recombinant cells” or equivalently “recombinant host cells” may be modified to express genes that are not found within the native (non-recombinant) form of the cell or may be modified to abnormally express native genes e.g., native genes may be overexpressed, underexpressed or not expressed at all. In exemplary embodiments, a “recombinant cell” or “recombinant host cell” is engineered to express a heterologous enzyme pathway capable of producing 3-HP. A recombinant cell may be derived from a microorganism or microbe such as a bacterium, proteobacterium, archaea, a virus, algae, or a fungus. In addition, a recombinant cell may be derived from a plant or an animal cell.

When used with reference to a polynucleotide, the term “recombinant” indicates that the polynucleotide has been modified by comparison to the native or naturally occurring form of the polynucleotide or has been modified by comparison to a naturally occurring variant of the polynucleotide. In an exemplary embodiment, a recombinant polynucleotide (or a copy or complement of a recombinant polynucleotide) is one that has been manipulated by the hand of man to be different from its naturally occurring form. Thus, in an exemplary embodiment, a recombinant polynucleotide is a mutant form of a native gene or a mutant form of a naturally occurring variant of a native gene wherein the mutation is made by intentional human manipulation e.g., made by saturation mutagenesis using mutagenic oligonucleotides, through the use of UV radiation, mutagenic chemicals, chemical synthesis etc. Such a recombinant polynucleotide might comprise one or more point mutations, deletions and/or insertions relative to the native or naturally occurring variant form of the gene. Similarly, a polynucleotide comprising a promoter operably linked to a second polynucleotide (e.g., a coding sequence) is a “recombinant” polynucleotide. Thus, a recombinant polynucleotide comprises polynucleotide combinations that are not found in nature. A recombinant protein (discussed supra) is typically one that is expressed from a recombinant polynucleotide, and recombinant cells, tissues, and organisms are those that comprise recombinant sequences (polynucleotide and/or polypeptide).

The term “vector,” as used herein, refers to a polynucleotide sequence that contains a gene of interest (e.g., it encodes one or more proteins or enzymes described herein) and a promoter operably linked to the ACA-hydrating enzyme and/or the oxidoreductase enzyme(s) polynucleotide sequence of interest. Once a polynucleotide sequence(s) encoding an ACA-hydrating enzyme and/or oxidoreductase enzyme(s) polypeptide has been prepared and isolated, various methods may be used to construct expression cassettes, vectors and other DNA constructs. The skilled artisan is well aware of the genetic elements that must be present on an expression construct/vector in order to successfully transform, select, and propagate the expression construct in host cells. Techniques for manipulation of nucleic acids such as subcloning nucleic acid sequences into expression vectors, labeling probes, DNA hybridization are well known in the art.

As used herein, the term “microbe” or “microorganism” refers generally to a microscopic organism. Microbes can be prokaryotic or eukaryotic. Exemplary prokaryotic microbes include e.g., bacteria (including γ-proteobacteria), archaea, cyanobacteria, etc. An exemplary proteobacterium is Escherichia coli. Exemplary eukaryotic microorganisms include e.g., yeast, protozoa, algae, etc. In exemplary embodiments, a “recombinant microbe” is a microbe that has been genetically altered and thereby expresses or encompasses a heterologous nucleic acid sequence and/or a heterologous peptide, polypeptide, or protein.

A microbe as used herein, may grow on a carbon source e.g., a simple carbon source. Typically, as used herein, a recombinant microbe, including a recombinant proteobacterium, comprises at least a ACA-hydrating enzyme or variant thereof having at least 85% sequence identity to SEQ ID NO: 1, 4, 14, 21, 24, 34, 41, 44, 54, 59, 62, and/or 72. The recombinant microbe may be a gamma proteobacterium (also known as a γ-proteobacterium), a cyanobacterium, a yeast, or an algae. In some embodiments, the recombinant proteobacterium may be Escherichia coli, Salmonella spp., Vibrio natriegens, Pseudomonas aeruginosa, Pseudomonas putida, Pseudomonas fluorescens, Xanthomonas axonopodis, Pseudomonas syringae, Xyella fastidiosa, Marinobacter aquaeolei, Yersinia pestis, or Vibrio cholerae. In some embodiments, the recombinant cyanobacterium may be Synechococcus elongatus PCC7942 or Synechocystis sp. PCC6803. In some embodiments, the recombinant yeast may be Saccharomyces cerevisiae, Scheffersomyces stipitis, Schizosaccharomyces pombe, Kluyveromyces marxianus, K. lactis, Pichia pastoris, Hansenula polymorpha, or Yarrowia lipolytica. In some embodiments, the recombinant algae may be Botryococcus braunii, Nannochloropsis gaditina, Chlamydomonas reinhardtii, Chlorella vulgaris, Spirulina platensis, Ostreococcus tauri, Phaeodactylum tricornutum, Symbiodinium sp., algal phytoplanktons, Saccharina japonica, Chlorococcum spp., and Spirogyra spp.

As used herein, the term “culture” typically refers to a liquid media comprising viable cells. In one embodiment, a culture comprises cells reproducing in a predetermined culture media under controlled conditions, for example, a culture of recombinant host cells grown in liquid media comprising a selected carbon source and nitrogen.

“Culturing” or “cultivation” refers to growing a population of recombinant host cells (e.g., recombinant microbes) under suitable conditions in a liquid or on a solid medium. In particular embodiments, culturing refers to the fermentative bioconversion of a substrate to an end-product. Culturing media are well-known and individual components of such culture media are available from commercial sources, e.g., under the Difco™ and BBL™ trademarks. In one non-limiting example, the aqueous nutrient medium is a “rich medium” comprising complex sources of nitrogen, salts, and carbon, such as Luria-Bertani (LB) medium, comprising 10 g/L of peptone and 10 g/L yeast extract of such a medium.

A “production host” or equivalently a “production host cell” is a cell used to produce products. As disclosed herein, a production host is typically modified to express or overexpress selected genes, or to have attenuated expression of selected genes. Thus, a production host or a “production host cell” is a recombinant host or equivalently a recombinant host cell. Non-limiting examples of production hosts include e.g., recombinant microbes as disclosed above. An exemplary production host is a recombinant proteobacterium comprising an ACA-hydrating enzyme or variant thereof.

As used herein, the terms “purify,” “purified,” or “purification” mean the removal or isolation of a molecule from its environment by, for example, isolation or separation. “Substantially purified” molecules are at least about 60% free (e.g., at least about 65% free, at least about 70% free, at least about 75% free, at least about 80% free, at least about 85% free, at least about 90% free, at least about 95% free, at least about 96% free, at least about 97% free, at least about 98% free, at least about 99% free) from other components with which they are associated. As used herein, these terms also refer to the removal of contaminants from a sample.

As used herein, the term “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO₂).

As used herein, the term “ACA” stands for acetylenecarboxylic acid. It is also known as propiolic acid and has the chemical structure:

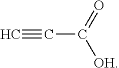

One of skill in the art is aware that ACA may be present in protonated or deprotonated form, thus “ACA” may also include an anion or salt thereof, and it is intended to be used interchangeably herein because one of skill in the art understands that the protonation state of compounds, such as ACA, may differ depending on the pH of the reaction. For example, the reactions described herein may take place with ACA in conjugate-base form (acetylenecarboxylate) instead of acetylenecarboxylic acid. Acetylenecarboxylic acid may be converted to acetylenecarboxylate (via loss of a proton) in a reaction with a pH range of 7-8. The reactions described herein may also take place with ACA in salt form, such as a potassium or sodium salt thereof.

As used herein, the term “MSA” stands for malonic semialdehyde and it has the following chemical structure:

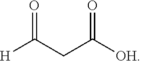

Similar to ACA, one of skill in the art is aware that MSA may be present in protonated or deprotonated form, thus “MSA” may also include an anion or salt thereof, and it is intended to be used interchangeably herein because one of skill in the art understands that the protonation state of MSA may differ depending on the pH of the reaction. For example, the reactions described herein may occur with MSA in conjugate-base form (malonate semialdehyde) instead of malonic semialdehyde. Malonic semialdehyde may be converted to malonate semialdehyde (via loss of a proton) in a reaction with a pH range of 7-8. The reactions described herein may also take place with MSA in salt form, such as a potassium or sodium salt thereof.

As used herein, the term “3-HP” stands for 3-hydroxypropionic acid and it has the following chemical structure:

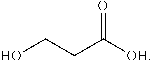

Similar to ACA and MSA, one of skill in the art is aware that 3-HP may be present in protonated or deprotonated form, thus “3-HP” may also include an anion or salt thereof, and it is intended to be used interchangeably herein because one of skill in the art understands that the protonation state of 3-HP may differ depending on the pH of the reaction. For example, 3-hydroxypropionic acid may be converted to 3-hydroxypropionate (via loss of a proton) in a reaction with a pH range of 7-8. The reactions described herein may also take place with 3-HP in salt form such as a potassium or sodium salt thereof.

### II. Enzymes

ACA-hydrating enzymes or variants thereof are disclosed herein for the production of 3-hydroxypropionic acid (3-HP) or an anion or salt thereof. The ACA-hydrating enzyme hydrates ACA or an anion or salt thereof to form a reaction product comprising MSA or an anion or salt thereof. Thus, the phrase “ACA-hydrating enzyme”, “ACA-hydrating enzyme variant” or “ACA-hydrating enzyme or variant thereof” refers to an enzyme capable of hydrating ACA or an anion or salt thereof. As used herein, an ACA-hydrating enzyme or variant thereof displays hydratase activity by producing MSA or an anion or salt thereof from ACA or an anion or salt thereof. For example, an ACA-hydrating enzyme or variant thereof may be a tautomerase, such as Cg10062 or a variant thereof, or cis-3-chloroacrylic acid dehalogenase (cis-CaaD) or a variant thereof.

In some embodiments, the tautomerase may be substantially free of decarboxylase activity. For example, the tautomerase may be substantially free of decarboxylase activity by producing less than 10%, less than 5%, less than 1%, or no acetaldehyde, for example.

The sequence of Cg10062 from Corynebacterium glutamicum was described in Poelarends et al. Biochemistry 47(31): 8139-47 (2008), which is incorporated herein by reference in its entirety. SEQ ID NO: 1 and 21 represent the full-length nucleotide and amino acid sequences of the Cg10062 from Corynebacterium glutamicum. SEQ ID NO: 41 and 59 represent full-length nucleotide and amino acid sequences of the Cg10062 from Corynebacterium glutamicum including a TEV protease recognition site and C-terminal His₆-tag added to the end of the sequence for experiments described herein. Thus, in some embodiments, the ACA-hydrating enzyme is a tautomerase, such as Cg10062. The Cg10062 may comprise SEQ ID NO: 21 or 59.

Additionally or alternatively, a variant of Cg10062 may be used and may comprise a sequence having a substitution at one or more amino acid positions of SEQ ID NO: 21 and/or 59, such as positions 28, 70, 73, 103, 114, etc. or a combination thereof. Cg10062 or a variant thereof may comprise one or more substitution mutations such as E114N, E114D, E114Q, H28A, R70A, R70K, R73A, R73K, Y103A, Y103F, E114A, E114D-Y103F, etc. or a combination thereof. In a particular embodiment, the variant of Cg10062 has the E114N mutation. SEQ ID NO: 22-33 represent amino acid sequences of a variant, non-naturally occurring Cg10062 enzyme. SEQ ID NO: 60-71 represent amino acid sequences of said variants including a TEV protease recognition site and C-terminal His₆-tag added to the end of the sequence for experiments described herein. In particular, SEQ ID NO: 24 and 62 represent an amino acid sequence of a novel Cg10062 variant comprising an E114N mutation. The Cg10062 (E114N) variant may have improved kinetic properties relative to a control and/or other Cg10062 variants. SEQ ID NO: 22 and 23 represent Cg10062 variants comprising E114Q and E114D mutations, respectively, compared to the wild-type Cg10062 sequence. SEQ ID NO: 60 and 61 represent Cg10062 variants comprising E114Q and E114D mutations, respectively, compared to the wild-type Cg10062 sequence, with an additional TEV protease recognition site and C-terminal His6-tag added to the end of the sequence for experiments described herein. Other Cg10062 variants may include the following mutations with respect to the wild-type Cg10062 SEQ ID NO: 21 (without TEV protease recognition site and C-terminal His₆-tag) and 59 (with TEV protease recognition site and C-terminal His₆-tag): H28A, R70A, R70K, R73A, R73K, Y103A, Y103F, E114A, E114D-Y103F, etc. H28A, R70A, R70K, R73A, R73K, Y103A, Y103F, E114A and E114D-Y103F correspond to SEQ ID NO: 25-33 (without TEV protease recognition site and C-terminal His₆-tag) and SEQ ID NO: 63-71 (with TEV protease recognition site and C-terminal His₆-tag). In one embodiment, the Cg10062 enzyme or variant thereof may have at least 85% sequence identity to SEQ ID NO: 1, 21, 41 or 59. In a further embodiment, the Cg10062 enzyme or variant thereof may have at least a 90% sequence identity to SEQ ID NO: 1, 21, 41, and/or 59, at least 95% sequence identity to SEQ ID NO: 1, 21, 41, and/or 59, at least 99% sequence identity to SEQ ID NO: 1, 21, 41, and/or 59, or is SEQ ID NO: 1, 21, 41 or 59 (e.g. 100% sequence homology).

The sequence of cis-CaaD from Coryneform bacterium was described in Poelarends et al. Biochemistry. 43(3): 759-72 (2004), which is incorporated herein by reference in its entirety. SEQ ID NO: 14 and 34 represent the full-length nucleotide and amino acid sequences of the cis-CaaD from Coryneform bacterium. SEQ ID NO: 54 and 72 represent the full-length nucleotide and amino acid sequences of the cis-CaaD from Coryneform bacterium, including a TEV protease recognition site and C-terminal His₆-tag added to the end of the sequence for experiments described herein. Thus, in some embodiments, the ACA-hydrating enzyme is a tautomerase, such as cis-3-chloroacrylic acid dehalogenase (cis-CaaD). The cis-CaaD may comprise amino acid SEQ ID NO: 34 or 72.

Additionally or alternatively, variants of cis-CaaD may also be used. In one embodiment, the cis-CaaD enzyme or variant thereof may have at least 85% sequence identity to SEQ ID NO: 14, 34, 54 or 72. In a further embodiment, the cis-CaaD enzyme or variant thereof may have at least a 90% sequence identity to SEQ ID NO: 14, 34, 54, and/or 72, at least 95% sequence identity to SEQ ID NO: 14, 34, 54, and/or 72, at least 99% sequence identity to SEQ ID NO: 14, 34, 54, and/or 72, or is SEQ ID NO: 14, 34, 54, or 72 (e.g. 100% sequence homology).

Without being bound by theory, it is possible that ACA-hydrating enzyme variants may synthesize MSA or an anion or salt thereof more efficiently than a control or wild-type ACA-hydrating enzyme. In some embodiments, enzymatic hydration may convert ACA or an anion or salt thereof to MSA or an anion or salt thereof without appreciable formation of acetaldehyde and/or CO₂. For example, an ACA-hydrating enzyme or variant thereof may generate less than 25%, less than 20%, less than 15%, less than 10%, less than 5%, or 0% acetaldehyde and/or CO₂ when converting ACA or an anion or salt thereof to MSA or an anion or salt thereof. Additionally or alternatively, a variant ACA-hydrating enzyme may convert ACA or an anion or salt thereof to MSA or an anion or salt thereof to produce at least 75%, at least 80%, at least 85%, at least 90%, at least 95%, or 100% MSA or an anion or salt thereof. In a specific embodiment, the reaction product comprising MSA or an anion or salt thereof may comprise about 95% or more MSA or an anion or salt thereof and about 5% or less of other reaction products.

Additionally, the reaction product comprising MSA formed from hydrating ACA may be substantially free of acetaldehyde and CO₂ For example, the reaction product comprising MSA or an anion or salt thereof may contain less than less than 25%, less than 20%, less than 15%, less than 10%, less than 5%, or 0% acetaldehyde and/or CO₂ and at least at least 75%, at least 80%, at least 85%, at least 90%, at least 95%, or 100% MSA or an anion or salt thereof. Additionally, the ACA-hydrating enzyme variant may not require metal cofactors, coenzymes, or CoA substrates. In certain embodiments, the variant ACA-hydrating enzyme may display enzymatic activity comparable to a control ACA-hydrating enzyme, but may generate only MSA from ACA-hydration. In a particular embodiment, the variant ACA-hydrating enzyme is Cg10062 (E114N) (SEQ ID NO: 24 or SEQ ID NO: 62). Additionally, the ACA-hydrating enzyme or variant thereof described herein may belong to EC (EC

The method described herein also comprises reacting the reaction product comprising MSA or an anion or salt thereof with one or more oxidoreductases in a redox reaction to produce 3-HP or an anion or salt thereof. As used herein, the term “oxidoreductase” refers to an enzyme that catalyzes oxidoreduction (redox) reactions. Redox reactions require an oxidoreductase enzyme to catalyze the transfer of electrons from one molecule (the oxidant) to another molecule (the reductant). Oxidoreductase enzymes may be oxidases or dehydrogenases. In some embodiments, redox reactions may use a pair of oxidoreductase enzymes to recycle/regenerate a cofactor. As used herein, the term “cofactor” refers to a non-protein chemical that assists with a biological chemical reaction, such as metal ions, organic compounds, or other chemicals. Examples of cofactors include NADPH, NADH, ATP, etc. In some embodiments, the pair of oxidoreductase enzymes may include 3-hydroxy acid dehydrogenase, such as YdfG, and a phosphite dehydrogenase, such as PTDH, or variants thereof, wherein the 3-hydroxy acid dehydrogenase or variant thereof is able to catalyze the reduction of MSA to 3-HP and the phosphite dehydrogenase or variant thereof catalyzes the NAD⁺-dependent conversion of phosphite to phosphate.

In some embodiments, the 3-hydroxy acid dehydrogenase is YdfG or a variant thereof having at least 85% sequence identity to SEQ ID NO: 17, 37, 57 and/or 75. In a further embodiment, the YdfG enzyme or variant thereof may have at least a 90% sequence identity to SEQ ID NO: 17, 37, 57 and/or 75, at least 95% sequence identity to SEQ ID NO: 17, 37, 57 and/or 75, at least 99% sequence identity to SEQ ID NO: 17, 37, 57 and/or 75, or is SEQ ID NO: 17, 37, 57 or 75 (e.g. 100% sequence homology).

In some embodiments, the phosphite dehydrogenase is PTDH or a variant thereof having at least 85% sequence identity to SEQ ID NO: 15, 35, 55, and/or 73. In a further embodiment, the PTDH enzyme or variant thereof may have at least a 90% sequence identity to SEQ ID NO: 15, 35, 55, and/or 73, at least 95% sequence identity to SEQ ID NO: 15, 35, 55, and/or 73, at least 99% sequence identity to SEQ ID NO: 15, 35, 55, and/or 73, or is SEQ ID NO: 15, 35, 55, or 73 (e.g. 100% sequence homology).

Additionally or alternatively, the pair of oxidoreductase enzymes may include a 3-hydroxyisobutyrate dehydrogenase, such as MmsB, and a soluble hydrogenase (SH) or variants of either, wherein the 3-hydroxyisobutyrate dehydrogenase or variant thereof is able to catalyze the reduction of MSA to 3-HP and the SH or variant thereof can catalyze the conversion of NAD⁺ to NADH.

In some embodiments the 3-hydroxyisobutyrate dehydrogenase is MmsB or a variant thereof having at least 85% sequence identity to SEQ ID NO: 18, 38, 58, and/or 76. In a further embodiment, the MmsB enzyme or variant thereof may have at least a 90% sequence identity to SEQ ID NO: 18, 38, 58, and/or 76, at least 95% sequence identity to SEQ ID NO: 18, 38, 58, and/or 76, at least 99% sequence identity to SEQ ID NO: 18, 38, 58, and/or 76, or is SEQ ID NO: 18, 38, 58, or 76 (e.g. 100% sequence homology).

SH is a multicomponent protein complex comprised of a hydrogenase module, which includes HoxH (WP_011154013.1) and HoxY (AAC06142.1), an NAD⁺ reductase module, which includes HoxF (WP_011154010.1) and HoxU (WP_011154011.1), and the nonessential HoxI (AAP85846.1) protein. In some embodiments, the SH is from Cupriavidus necator HF210 expressing the pGE771 plasmid. Methods for preparing SH from Cupriavidus necator HF210 containing the pGE771 plasmid are known in the art from Lenz, O. Meth. Enzymol. (2018) 613, 117-151 and also Horch, Marius. Structure-function Relationships of Metalloenzymes. PhD thesis, Technical University of Berlin, Berlin, Jun. 3, 2015, which is incorporated herein by reference in its entirety. Plasmid pGE771 includes all the genes necessary for expression of functional SH including those for the structural proteins HoxF (WP_011154010.1), HoxU (WP_011154011.1), HoxY (AAC06142.1), HoxH (WP_011154013.1), and Hoxl (AAP85846.1). The hoxF (WP_011154010.1) structural gene may be amended to include a tag, such as a Strep-tagII, on the amino terminus to facilitate protein purification. Plasmid pGE771 also includes hoxW (encodes protein accession no. WP_011154014.1), which encodes a hydrogenase-specific protease, as well as hypA2 (encodes protein accession no. AAP85847.1), hypB2 (encodes protein accession no. AAP85848.1), hypF2 (encodes protein accession no. AAP85849.1), hypC (encodes protein accession no. CAA49733.1), hypD (encodes protein accession no. CAA49734.1), hypE (encodes protein accession no. CAA49735.1), and hypX (encodes protein accession no. WP_011153943), which are responsible for SH assembly and insertion of the [NiFe] catalytic center. The hoxA gene (encodes protein accession no. AAP85775.1) is also included on pGE771 to enable HoxA-mediated expression of the hox operon.

In some further embodiments, a pair of oxidoreductase enzymes may recycle a cofactor, such as NADPH or NADH. In a further embodiment, YdfG and PTDH may be involved in a redox reaction to generate 3-HP and recycle the cofactor NADPH. Alternatively, MmsB and SH may be involved in a redox reaction to generate 3-HP and cycle the cofactor NADH. In some embodiments, the oxidoreductase enzyme(s) may belong to E.C.1.

### III. Synthesis of a Cetylene Carboxylic Acid (ACA) and Acetylenedicarboxylic Acid (ADCA)

The inventors have identified ACA as a novel starting material for 3-HP synthesis. It should be noted that acetylenedicarboxylic acid (ADCA) may be decarboxylated to ACA for use as a starting material for 3-HP synthesis as well. ACA and ADCA may be synthesized via acetylene from CH₄ and CO₂, both of which are greenhouse gases whose increasing atmospheric concentrations are cause for pressing environmental concern. In some embodiments, CH₄ may be obtained from fossil fuel-derived natural gas or from renewable biogas and/or CO₂ may be obtained as a product of combustion and aerobic metabolism of sugars. Thus, in some embodiments, the ACA and/or ADCA generated from CH₄ and CO₂ may be used as a starting material to produce 3-HP.

In some embodiments, ACA, ADCA, or an anion or salt thereof may be synthesized by dehydrodimerization of CH₄ to produce acetylene, wherein the acetylene is reacted with CO₂ to produce ACA, ADCA, or an anion or salt thereof (FIG. 2). It is possible acetylene may vary in selectivity for ACA and ADCA depending on the reaction conditions. In some embodiments, acetylene may have 50%, 60%, 70%, 80% 90% or 100% selectivity for ACA. It is also possible that acetylene may have different rates of conversion to ACA depending on the reaction conditions. In some embodiments acetylene may have 50%, 60%, 70%, 80% 90% or 100% rate of conversion to ACA. In a particular embodiment, acetylene may have 90% selectivity for ACA and 70% rate of conversion to ACA.

### IV. Recombinant Microbes/Cells Comprising ACA-Hydrating Enzymes and Oxidoreductase Enzymes

As discussed above, 3-HP or an anion or salt thereof may be generated by converting ACA or an anion or salt thereof to MSA or an anion or salt thereof via an ACA-hydrating enzyme or variant thereof, followed by a redox reaction via one or more oxidoreductase enzymes to convert the MSA or an anion or salt thereof to 3-HP or an anion or salt thereof. Thus, in one embodiment, a recombinant microbe comprising an ACA-hydrating enzyme or variant thereof having at least 85% sequence identity to SEQ ID NO: 1, 4, 14, 21, 24, 34, 41, 44, 54, 59, 62, and/or 72 is disclosed herein. In another embodiment, a recombinant microbe comprising one or more oxidoreductase enzymes having at least 85% sequence identity to SEQ ID NO: 15, 17, 18, 35, 37, 38, 55, 57, 58, 73, 75, and/or 76 is disclosed herein. In a further embodiment, a recombinant microbe comprising an ACA-hydrating enzyme or variant thereof having at least 85% sequence identity to SEQ ID NO: 1, 4, 14, 21, 24, 34, 41, 44, 54, 59, 62, and/or 72 and one or more oxidoreductase enzymes having at least 85% sequence identity to SEQ ID NO: 15, 17, 18, 35, 37, 38, 55, 57, 58, 73, and/or 76 is disclosed herein.

For example, the ACA-hydrating enzyme or variant thereof may comprise a sequence having about 85% sequence identity, at least a 90% sequence identity, at least a 95% sequence identity, or at least a 99% sequence identity to a sequence of SEQ ID NO: 1, 4, 14, 21, 24, 34, 41, 44, 54, 59, 62, and/or 72. In particular, the ACA-hydrating enzyme or variant thereof may comprise a sequence of SEQ ID NO: 1, 4, 14, 21, 24, 34, 41, 44, 54, 59, 62, or 72. In an example embodiment, the recombinant cell is genetically engineered to express a variant tautomerase comprising the amino acid sequence of SEQ ID NO: 4 (Cg10062 El14N variant).

Additionally, the one or more oxidoreductase enzyme may comprise a sequence(s) having about 85% sequence identity, at least a 90% sequence identity, at least a 95% sequence identity, or at least a 99% sequence identity to a sequence of SEQ ID NO: 15, 17, 18, 35, 37, 38, 55, 57, 58, 73, 75, and/or 76. In particular, the one or more oxidoreductase enzyme may comprise a sequence of SEQ ID NO: 15, 17, 18, 35, 37, 38, 55, 57, 58, 73, 75, or 76. The recombinant microbe may comprise any combination of ACA-hydrating enzymes or variants thereof and oxidoreductase enzymes described herein.

The recombinant microbe described herein may be a bacterium, yeast, or an algae. In one embodiment, the recombinant microbe is a recombinant proteobacterium, such as a γ-proteobacterium. The γ-proteobacterium may be Escherichia coli, Salmonella spp., Vibrio natriegens, Pseudomonas aeruginosa, Pseudomonas putida, Pseudomonas fluorescens, Xanthomonas axonopodis, Pseudomonas syringae, Xyella fastidiosa, or Marinobacter aquaeolei. In a particular, the γ-proteobacterium may be Escherichia coli.

Additionally or alternatively, the recombinant microbe may be a cyanobacterium such as Synechococcus elongatus PCC7942 or Synechocystis sp. PCC6803.

Additionally or alternatively, the recombinant microbe may be a yeast such as Saccharomyces cerevisiae, Scheffersomyces stipitis, Schizosaccharomyces pombe, Kluyveromyces marxianus, K. lactis, Pichia pastoris, Hansenula polymorpha, and Yarrowia lipolytica or an algae such as Botryococcus braunii, Nannochloropsis gaditina, Chlamydomonas reinhardtii, Chlorella vulgaris., Spirulina platensis, Ostreococcus tauri, Phaeodactylum tricornutum, Symbiodinium sp., algal phytoplanktons, Saccharina japonica, Chlorococum spp., and Spirogyra spp.

Various amounts of MSA or an anion or salt thereof may be produced from the recombinant microbes described herein. In some embodiments, the amount of MSA produced may be more than what is produced by a control. As discussed herein, a recombinant microbe may synthesize MSA. In particular, a recombinant microbe may synthesize 5 wt % or more, 10 wt % or more, 15 wt % or more, 20 wt % or more, 25 wt % or more, 30 wt % or more, 35 wt % or more, 40 wt % or more, 45 wt % or more, or 50 wt % or more MSA, than a control recombinant microbe (e.g. a recombinant microbe comprising a non-genetically manipulated ACA-hydrating enzyme).

Along with MSA or an anion or salt thereof, various amounts of 3-HP or an anion or salt thereof may be produced from the recombinant microbes described herein. In some embodiments, the amount of 3-HP produced may be more than what is produced by a control. As discussed herein, a recombinant microbe may synthesize 3-HP. In particular, a recombinant microbe may synthesize 5 wt % or more, 10 wt % or more, 15 wt % or more, 20 wt % or more, 25 wt % or more, 30 wt % or more, 35 wt % or more, 40 wt % or more, 45 wt % or more, or 50 wt % or more 3-HP, than a control recombinant microbe (e.g. a recombinant microbe comprising non-genetically manipulated oxidoreductase enzyme(s)).

The enzymes described herein may be heterologous to the host cell or a production host cell. Additionally, the enzymes described herein may be native or non-native to the host cell or a production host cell. In some embodiments, the enzymes described herein may be heterologous and native (e.g. a wild-type enzyme produced within the host cell). Alternatively, the enzymes may be heterologous and non-native (e.g. a variant enzyme produced within the cell). In some embodiments, the host cell may encode a heterologous, non-native ACA-hydrating enzyme and a heterologous, non-native oxidoreductase enzyme(s). In a particular embodiment, the host cell may encode Cg10062 (E114N) (e.g. heterologous and non-native enzyme) and YdfG (e.g. heterologous and native enzyme).

In some embodiments, the host cell or production host cell may encode one oxidoreductase enzyme. Additionally, the host cell or production host cell may encode two oxidoreductase enzymes. One of the two oxidoreductase enzymes may function to recycle/regenerate a cofactor. Additionally or alternatively, the host cell or production host cell may recycle/regenerate a cofactor using one or more endogenous enzymes.

In some exemplary embodiments, the host cell or a production host cell (e.g., a recombinant microbe or recombinant proteobacterium, cyanobacterium or algae) may further comprise genetic manipulations and alterations to enhance or otherwise fine tune the production of MSA and/or 3-HP. The optional genetic manipulations may be used interchangeably from one host cell to another, depending on what other heterologous enzymes and what native enzymatic pathways are present in the host cell.

### V. Compositions

Further provided herein are compositions for generating MSA and/or 3-HP, such as reaction mixes and intermediate compositions; and also end-product compositions which may be generated by the method described herein. Therefore, a composition is described herein produced by reacting ACA or an anion or salt thereof with an ACA-hydrating enzyme. The composition described herein may comprise at least 95% MSA or an anion or salt thereof and less than 5% acetaldehyde and CO₂. All percentages used herein are with respect to the total weight of the composition.

A composition described herein may comprise less than 10 wt % of MSA. Additionally or alternatively, the composition may be substantially free of MSA. For example, the composition may comprise less than about 5 wt %, less than about 4 wt %, less than about 3 wt %, less than about 2 wt %, less than about 1 wt %, less than about 0.5%, less than about 0.1 wt %, less than about 0.05 wt %, less than about 0.01 wt %, or about 0 wt % (e.g., no) MSA relative to the total weight of the composition. Alternatively, the composition may comprise more than more than 1 wt %, more than 2 wt %, more than 3 wt %, more than 4 wt %, more than 5 wt %, more than 10 wt %, more than 15 wt %, more than 20 wt %, more than 25 wt %, more than 30 wt %, more than 35 wt %, more than 40 wt %, more than 45 wt %, or more than 50 wt % of MSA relative to the total weight of the composition. Moreover, the composition may comprise more than 1 wt % of MSA relative to the total weight of the composition.

Additionally, the composition may be considered substantially free of acetaldehyde and/or CO₂. For example, the composition may comprises less than about 5 wt %, less than about 4 wt %, less than about 3 wt %, less than about 2 wt %, less than about 1 wt %, less than about 0.5 wt %, less than about 0.1 wt %, less than about 0.05 wt %, less than about 0.01 wt %, or about 0 wt % of the total amount of acetaldehyde and/or CO 2 relative to the total weight of the composition.

Additionally or alternatively to MSA, the composition described herein may comprise less than 10 wt % of 3-HP. Additionally or alternatively, the composition may be substantially free of 3-HP. For example, the composition may comprise less than about 5 wt %, less than about 4 wt %, less than about 3 wt %, less than about 2 wt %, less than about 1 wt %, less than about 0.5%, less than about 0.1 wt %, less than about 0.05 wt %, less than about 0.01 wt %, or about 0 wt % (e.g., no) 3-HP relative to the total weight of the composition. Alternatively, the composition may comprise more than 1 wt %, more than 2 wt %, more than 3 wt %, more than 4 wt %, more than 5 wt %, more than 10 wt %, more than 15 wt %, more than 20 wt %, more than 25 wt %, more than 30 wt %, more than 35 wt %, more than 40 wt %, more than 45 wt %, or more than 50 wt % of 3-HP relative to the total weight of the composition. Moreover, the composition may comprise more than 1 wt % of 3-HP relative to the total weight of the composition.

Additionally, the composition may comprise an ACA-hydrating enzyme or variant thereof. In one embodiment, the ACA-hydrating enzyme or variant thereof may have at least 85% sequence identity to SEQ ID NO: 1, 4, 14, 21, 24, 34, 41, 44, 54, 59, 62, and/or 72. In a specific embodiment, the ACA-hydrating enzyme or variant thereof may comprise a sequence having about 85% sequence identity, at least a 90% sequence identity, at least a 95% sequence identity, or at least a 99% sequence identity to a sequence of SEQ ID NO: 1, 4, 14, 21, 24, 34, 41, 44, 54, 59, 62, and/or 72. In particular, the ACA-hydrating enzyme or variant thereof may comprise a sequence of SEQ ID NO: 1, 4, 14, 21, 24, 34, 41, 44, 54, 59, 62, or 72. For example, the composition may comprise more than more than 1 wt %, more than 2 wt %, more than 3 wt %, more than 4 wt %, more than 5 wt %, more than 10 wt %, more than 15 wt %, more than 20 wt %, more than 25 wt %, more than 30 wt %, more than 35 wt %, more than 40 wt %, more than 45 wt %, or more than 50 wt % of an ACA-hydrating enzyme relative to the total weight of the composition. Moreover, the composition may comprise more than 1 wt % of an ACA-hydrating enzyme or variant thereof relative to the total weight of the composition.

Additionally, the composition may comprise one or more oxidoreductase enzymes. In one embodiment, the one or more oxidoreductase enzymes may have at least 85% sequence identity to SEQ ID NO: 15, 17, 18, 35, 37, 38, 55, 57, 58, 73, 75, and/or 76. In a specific embodiment, the one or more oxidoreductase enzymes may comprise a sequence having about 85% sequence identity, at least a 90% sequence identity, at least a 95% sequence identity, or at least a 99% sequence identity to a sequence of SEQ ID NO: 15, 17, 18, 35, 37, 38, 55, 57, 58, 73, 75, and/or 76. In particular, the one or more oxidoreductase enzymes may comprise a sequence of SEQ ID NO: 15, 17, 18, 35, 37, 38, 55, 57, 58, 73, 75, or 76. For example, the composition may comprise more than more than 1 wt %, more than 2 wt %, more than 3 wt %, more than 4 wt %, more than 5 wt %, more than 10 wt %, more than 15 wt %, more than 20 wt %, more than 25 wt %, more than 30 wt %, more than 35 wt %, more than 40 wt %, more than 45 wt %, or more than 50 wt % of one or more oxidoreductase enzymes relative to the total weight of the composition. Moreover, the composition may comprise more than 1 wt % of one or more oxidoreductase enzymes relative to the total weight of the composition.

The composition may comprise any combination of ACA-hydrating enzymes or variants thereof and oxidoreductase enzymes described herein. For example, one composition could be set up to facilitate the reaction of ACA or an anion or salt thereof to MSA or an anion or salt thereof, which may include a wt % of ACA and a wt % of an ACA-hydrating enzyme. In another example, the composition could be set up to facilitate the reaction of MSA or an anion or salt thereof to 3-HP or an anion or salt thereof, which may include a wt % of MSA and a wt % of one or more oxidoreductase enzymes. In yet another example, the composition could be set up to facilitate both reactions (a 2-step reaction), which may include a wt % of ACA, a wt % of an ACA-hydrating enzyme, and a wt % of one or more oxidoreductase enzymes. In a particular embodiment, the composition may comprise a Cg10062 variant (ACA-hydrating enzyme variant). Additionally or alternatively, the composition may comprise YdfG and PTDH (oxidoreductase enzyme pair). Additionally or alternatively, the composition may comprise MmsB and SH (oxidoreductase enzyme pair). Alternatively, the composition may only include one oxidoreductase enzyme.

Additionally, the composition may comprise a cofactor as described herein. In a particular embodiment, the composition may comprise 1 wt %, more than 2 wt %, more than 3 wt %, more than 4 wt %, more than 5 wt %, more than 10 wt %, more than 15 wt %, more than 20 wt %, more than 25 wt %, more than 30 wt %, more than 35 wt %, more than 40 wt %, more than 45 wt %, or more than 50 wt % of a cofactor relative to the total weight of the composition. Moreover, the composition may comprise more than 1 wt % of a cofactor relative to the total weight of the composition.

In addition to the above, the composition may comprise an ACA-hydrating enzyme or variant thereof, one or more oxidoreductase enzymes described herein, and a cofactor described herein. For example, in a particular embodiment, the composition may comprise a Cg10062 variant (ACA-hydrating enzyme variant), YdfG and PTDH (oxidoreductase enzyme pair) and NADPH (cofactor). In another embodiment, the composition may comprise a Cg10062 variant (ACA-hydrating enzyme), MmsB and SH (oxidoreductase enzyme pair) and NADH (cofactor). In a further embodiment, the composition may comprise a Cg10062 variant, MmsB and NADH. The composition may further include ACA or an anion or salt thereof, to which the reaction mix is added.

Additionally or alternatively, the composition may be prepared by culturing a recombinant microbe described herein, such as a recombinant microbe comprising a heterologous ACA-hydrating enzyme or variant thereof, wherein the heterologous ACA-hydrating enzyme or variant thereof may have at least 85% sequence identity to SEQ ID NO: 1, 4, 14, 21, 24, 34, 41, 44, 54, 59, 62, and/or 72. In a further embodiment, the composition may be prepared by culturing a recombinant microbe described herein, such as a recombinant microbe comprising one or more oxidoreductase enzymes, wherein the one or more heterologous oxidoreductase enzymes may have at least 85% sequence identity to SEQ ID NO: 15, 17, 18, 35, 37, 38, 55, 57, 58, 73, 75, and/or 76.

Additionally or alternatively, the recombinant microbe used in the composition may be engineered to express an ACA-hydrating enzyme and/or variant thereof and one or more oxidoreductase enzymes as described herein. In some embodiments, the enzymes described herein may be exogenous to the host cell or production host cell described herein. For example, the enzyme(s) may be added to the culture/cell/assay (without being produced by the host cell). In one embodiment, an ACA-hydrating enzyme may be added to an assay which also includes a recombinant host cell that encodes one or more oxidoreductase enzymes. In a particular embodiment, the Cg10062 (E114N) enzyme may be added to an assay that also includes a recombinant host cell that encodes YdfG.

### VI. Nucleotide/Amino Acid Sequences and Vectors

SEQ ID NO: 21-34, 36, 59-72, and 74 comprise amino acid sequences of enzymes wherein the initial methionine is post translationally removed. For example, SEQ ID NO: 1 represents the nucleic acid sequence of wild-type Cg10062 and includes the initial nucleotides “ATG” which translate to amino acid “M” (e.g., methionine). SEQ ID NO: 21 and 59 represent the amino acid sequence of wild-type Cg10062 and do not include the initial “M” due to the post-translation removal.

Many nucleotide and amino acid sequences used for experiments described herein were constructed with a TEV protease recognition site and C-terminal His₆-tag at the end of the sequence. The TEV protease recognition site and C-terminal His₆-tag are connected via two amino acids. The His₆-tag may be added for affinity purification. The added TEV protease recognition site and C-terminal His₆-tag nucleotide and amino acid sequences correspond to SEQ ID NO: 20 and SEQ ID NO: 40, respectively. Nucleotide and amino acid sequences that include the TEV protease recognition sequence plus C-terminal His₆-tag are presented in SEQ ID NO: 41-54, 56-58 and 59-72, 74-76, respectively. Although experiments described herein were carried out with sequences which include the TEV protease recognition sequence and C-terminal His₆-tag, it should be appreciated that the method described herein may also be carried out with sequences that do not include the TEV protease recognition sequence plus His₆-tag.

Additionally, PTDH nucleotide and amino acid sequences used for experiments described herein were previously engineered with an N-terminal His₆-tag from pET-15b vector. The N-terminal His₆-tag nucleotide and amino acid sequences correspond to SEQ ID NO: 19 and 39, respectively. Nucleotide and amino acid sequences that include the N-terminal His₆-tag are presented in SEQ ID NO: 55 and SEQ ID NO: 73.

Described herein are a nucleotide sequences that encode an ACA-hydrating enzyme or variant thereof having at least 85%, at least 90%, at least 95%, or 100% sequence identity to any one of SEQ ID NO: 1-14 and 41-54 and a vector comprising the nucleotide sequence that encodes the ACA-hydrating enzyme having at least 85%, at least 90%, at least 95%, or 100% sequence identity to any one of SEQ ID NO: 1-14 and 41-54. For example, the nucleotide sequence encoding the ACA-hydrating enzyme or variant thereof having at least 85%, at least 90%, at least 95%, or 100% sequence identity to any one of SEQ ID NO: 1-14 and 41-54 and/or a vector comprising the nucleotide sequence encoding the ACA-hydrating enzyme or variant thereof having at least 85%, at least 90%, at least 95%, or 100% sequence identity to any one of SEQ ID NO: 1-14 and 41-54 may be constructed by methods well known in the art. The nucleotide sequence encoding the ACA-hydrating enzyme or variant thereof having at least 85%, at least 90%, at least 95%, or 100% sequence identity to any one of SEQ ID NO: 1-14 and 41-54 may be operably linked to one or more heterologous regulatory elements. Where the vector comprises a nucleotide sequence encoding the ACA-hydrating enzyme or variant thereof recited above, the vector may comprise a single heterologous regulatory element that directs expression of both ACA-hydrating enzyme or variant thereof and additional elements or multiple heterologous regulatory elements that independently directs expression of each of the ACA-hydrating enzymes or variants thereof and one or more of the additional elements encoded by the vector.

Also described herein are nucleotide sequences encoding the one or more oxidoreductase enzyme having at least 85%, at least 90%, at least 95%, or 100% sequence identity to any one of SEQ ID NO: 15, 17-18, 55, 57-58 and a vector comprising the nucleotide sequence that encodes the one or more oxidoreductase enzyme having at least 85%, at least 90%, at least 95%, or 100% sequence identity to any one of SEQ ID NO: 15, 17-18, 55, 57-58. For example, the nucleotide sequence encoding the one or more oxidoreductase enzyme having at least 85%, at least 90%, at least 95%, or 100% sequence identity to any one of SEQ ID NO: 15, 17-18, 55, 57-58 and/or a vector comprising the nucleotide sequence encoding the one or more oxidoreductase enzyme having at least 85%, at least 90%, at least 95%, or 100% sequence identity to any one of SEQ ID NO: 15, 17-18, 55, 57-58 may be constructed by methods well known in the art.

The nucleotide sequence(s) encoding the one or more oxidoreductase enzyme having at least 85%, at least 90%, at least 95%, or 100% sequence identity to any one of SEQ ID NO: 15, 17-18, 55, 57-58 may be operably linked to one or more heterologous regulatory elements. Where the vector comprises a nucleotide sequence encoding the one or more oxidoreductase enzyme(s) recited above, the vector may comprise a single heterologous regulatory element that directs expression of both oxidoreductase enzyme(s) and additional elements or multiple heterologous regulatory elements that independently directs expression of each of the oxidoreductase enzyme(s) and one or more of the additional elements encoded by the vector.

In some embodiments, the vector may comprise a nucleotide sequence that encodes an ACA-hydrating enzyme or variant thereof having at least 85%, at least 90%, at least 95%, or 100% sequence identity to SEQ ID NO: SEQ ID NO: 21-34 and 59-72 as well as the one or more oxidoreductase enzyme(s) having at least 85%, at least 90%, at least 95%, or 100% sequence identity to any one of SEQ ID NO: 35, 37-38, 73, and 75-76.

As mentioned above, the nucleotide sequences described herein may encode proteins such as ACA-hydrating enzymes and oxidoreductase enzymes. ACA-hydrating enzyme amino acid sequences or variants thereof may have at least 85%, at least 90%, at least 95%, or 100% sequence identity to any one of SEQ ID NO: 21-34 and 59-72. Oxidoreductase enzyme amino acid sequences or variants thereof may have at least 85%, at least 90%, at least 95%, or 100% sequence identity to any one of SEQ ID NO: 35, 37-38, 73, and 75-76.

Therefore, a non-naturally occurring variant tautomerase including an amino acid sequence of SEQ ID NO: 24 or SEQ ID NO: 62 is described herein. Also described herein is a vector comprising a nucleotide sequence encoding a variant tautomerase including an amino acid sequence of SEQ ID NO: 24 or 62. Additionally, a recombinant cell is described herein that is genetically engineered to express a variant tautomerase including an amino acid sequence of SEQ ID NO: 24 or 62. The variant tautomerase described herein may be a variant of Cg10062. The variant of Cg10062 may include one or more of the following mutations: H28A, R70A, R70K, R73A, R73K, Y103A, Y103F, E114A, E114D, E114N, and E114Q. In an example embodiment, the variant tautomerase is Cg10062 (E114E). In some embodiments, the vector and/or recombinant microbe described herein may encode Cg10062 (E114N).

Additionally, the recombinant cell described above may be genetically engineered to express one or more oxidoreductases comprising an amino acid sequence having at least 85% sequence identity to SEQ ID NO: 35, 37, 38, 73, 75, or 76.

As noted above, a polynucleotide or polypeptide may be overexpressed using methods well known in the art. In some embodiments, overexpression of a polypeptide is achieved by the use of an exogenous regulatory element. The term “exogenous regulatory element” generally refers to a regulatory element originating outside of the host cell. However, in certain embodiments, the term “exogenous regulatory element” may refer to a regulatory element derived from the host cell whose function is replicated or usurped for the purpose of controlling the expression of an endogenous polypeptide. For example, if the host cell is an E. coli cell, and the YdfG enzyme or variant thereof is encoded by an endogenous gene, then expression of the endogenous gene may be controlled by a promoter derived from another E. coli gene or from another species entirely.

In some embodiments, the exogenous regulatory element is a chemical compound, such as a small molecule. As used herein, the term “small molecule” refers to a substance or compound having a molecular weight of less than about 1,000 g/mol.

In some embodiments, the exogenous regulatory element is an expression control sequence which is operably linked to the endogenous gene by recombinant integration into the genome of the host cell. In certain embodiments, the expression control sequence is integrated into a host cell chromosome by homologous recombination using methods well known in the art (e.g., Datsenko et al., Proc. Natl. Acad. Sci. U.S.A., 97(12): 6640-6645 (2000)).

In some embodiments, a vector described herein comprises a promoter operably linked to the polynucleotide sequence. In certain embodiments, the promoter is a developmentally-regulated promoter, an organelle-specific promoter, a tissue-specific promoter, an inducible promoter, a constitutive promoter, or a cell-specific promoter.

In some embodiments, a vector described herein comprises at least one sequence such as (a) an expression control sequence (or regulatory element) operatively coupled to the polynucleotide sequence; (b) a selection marker operatively coupled to the polynucleotide sequence; (c) a marker sequence operatively coupled to the polynucleotide sequence; (d) a purification moiety operatively coupled to the polynucleotide sequence; (e) a secretion sequence operatively coupled to the polynucleotide sequence; and (f) a targeting sequence operatively coupled to the polynucleotide sequence.

The expression vectors described herein include a polynucleotide sequence described herein in a form suitable for expression of the polynucleotide sequence in a host cell. It will be appreciated by those skilled in the art that the design of the expression vector can depend on such factors as the choice of the host cell to be transformed, the level of expression of polypeptide desired, etc. The expression vectors described herein may be introduced into host cells to produce polypeptides, including fusion polypeptides, encoded by the polynucleotide sequences as described herein.

Expression of genes encoding polypeptides in prokaryotes, for example, E. coli, is most often carried out with vectors containing constitutive or inducible promoters directing the expression of either fusion or non-fusion polypeptides. Fusion vectors add a number of amino acids to a polypeptide encoded therein, usually to the amino- or carboxy-terminus of the recombinant polypeptide. Such fusion vectors typically serve one or more of the following three purposes: (1) to increase expression of the recombinant polypeptide; (2) to increase the solubility of the recombinant polypeptide; and (3) to aid in the purification of the recombinant polypeptide by acting as a ligand in affinity purification. Often, in fusion expression vectors, a proteolytic cleavage site is introduced at the junction of the fusion moiety and the recombinant polypeptide. This enables separation of the recombinant polypeptide from the fusion moiety after purification of the fusion polypeptide. Examples of such enzymes, and their cognate recognition sequences, include Factor Xa, thrombin, and enterokinase. Exemplary fusion expression vectors include pGEX (Pharmacia Biotech, Inc., Piscataway, NJ; Smith et al., Gene, 67: 31-40 (1988)), pMAL (New England Biolabs, Beverly, MA), and pRITS (Pharmacia Biotech, Inc., Piscataway, N.J.), which fuse glutathione S-transferase (GST), maltose E binding protein, or protein A, respectively, to the target recombinant polypeptide.

Suitable expression systems for both prokaryotic and eukaryotic cells are well known in the art; see, e.g., Sambrook et al., “Molecular Cloning: A Laboratory Manual,” second edition, Cold Spring Harbor Laboratory (1989). Examples of inducible, non-fusion E. coli expression vectors include pTrc (Amann et al., Gene, 69: 301-315 (1988)) and pET-11d (Studier et al., Gene Expression Technology: Methods in Enzymology 185, Academic Press, San Diego, CA, pp. 60-89 (1990)). In certain embodiments, a polynucleotide sequence of the invention is operably linked to a promoter derived from bacteriophage T5. Examples of vectors for expression in yeast include pYepSec1 (Baldari et al., EMBO J., 6: 229-234 (1987)), pMFa (Kurjan et al., Cell, 30: 933-943 (1982)), pJRY88 (Schultz et al., Gene, 54: 113-123 (1987)), pYES2 (Invitrogen Corp., San Diego, CA), and picZ (Invitrogen Corp., San Diego, CA). Baculovirus vectors available for expression of proteins in cultured insect cells (e.g., Sf9 cells) include, for example, the pAc series (Smith et al., Mol. Cell Biol., 3: 2156-2165 (1983)) and the pVL series (Lucklow et al., Virology, 170: 31-39 (1989)). Examples of mammalian expression vectors include pCDM8 (Seed, Nature, 329: 840 (1987)) and pMT2PC (Kaufman et al., EMBO J., 6: 187-195 (1987)).

Vectors may be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in, for example, Sambrook et al. (supra).

For stable transformation of bacterial cells, it is known that, depending upon the expression vector and transformation technique used, only a small fraction of cells will take-up and replicate the expression vector. In order to identify and select these transformants, a gene that encodes a selectable marker (e.g., resistance to an antibiotic) can be introduced into the host cells along with the gene of interest. Selectable markers include those that confer resistance to drugs such as, but not limited to, ampicillin, kanamycin, chloramphenicol, spectinomycin, or tetracycline. Nucleic acids encoding a selectable marker may be introduced into a host cell on the same vector as that encoding a polypeptide described herein or can be introduced on a separate vector. Cells stably transformed with the introduced nucleic acid may be identified by growth in the presence of an appropriate selection drug.

Similarly, for stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to an antibiotic) may be introduced into the host cells along with the gene of interest. Preferred selectable markers include those which confer resistance to drugs, such as G418, hygromycin, and methotrexate. Nucleic acids encoding a selectable marker may be introduced into a host cell on the same vector as that encoding a polypeptide described herein or may be introduced on a separate vector. Cells stably transfected with the introduced nucleic acid may be identified by growth in the presence of an appropriate selection drug.

Also described herein are nucleotide sequences used as primers (SEQ ID NOs: 77-93). The primers described herein may be used for the construction of Cg10062 mutants. The primers may contain restriction sites to aid in cleavage and integration. For example, the gene encoding YdfG may be amplified from E. coli W3110 genomic DNA using primers with NdeI and XhoI restriction sites at the 5′ and 3′ positions, respectively.

### VII. Methods of Producing MSA and 3-HP

In addition to the recombinant microbes and compositions described above, methods of producing MSA or an anion or salt thereof and/or 3-HP or an anion or salt thereof are described herein. The disclosed invention provides methods of generating 3-HP or an anion or salt thereof in vitro and/or in vivo.

For example, methods of producing MSA or an anion or salt thereof and/or 3-HP or an anion or salt thereof are described herein, where ACA or an anion or salt thereof may be reacted with an ACA-hydrating enzyme to form a reaction product comprising MSA or an anion or salt thereof, and said reaction product may be reacted with one or more oxidoreductase enzymes in a redox reaction to generate 3-HP or an anion or salt thereof. Additionally, the one or more oxidoreductases may recycle a cofactor, such as NADPH or NADH.

The ACA-hydrating enzyme may be a tautomerase such as Cg10062 or a variant thereof capable of hydrating ACA or an anion or salt thereof; or cis-CaaD or a variant thereof capable of hydrating ACA or an anion or salt thereof. In some embodiments, the tautomerase used in the methods described herein may be substantially free of decarboxylase activity. In some embodiments, the tautomerase may be a non-decarboxylating variant and may not produce acetaldehyde. Therefore, the tautomerase may have hydratase-only activity and may only produce MSA. In a particular embodiment, the Cg10062 (E114N) (SEQ ID NO: 24 and SEQ ID NO: 62) variant may be a non-decarboxylating variant and may not produce acetaldehyde. Therefore, the variant may have hydratase-only activity and may only produce MSA.

The ACA-hydrating enzyme may be a Cg10062 enzyme or variant thereof that has at least 85%, preferably 90%, sequence identity to SEQ ID NO: 1, 4, 21, 24, 41, 44, 59, and/or 62. Additionally or alternatively, the ACA-hydrating enzyme may be a cis-Caad enzyme that has at least 85%, preferably 90%, sequence identity to SEQ ID NO: 14, 34, 54, and/or 72.

In additional embodiments, the variant of Cg10062 may comprise at least one mutation at an amino acid position corresponding to amino acid position 28, 70, 73, 103 and 114. For example, the variant may have one or more of the following mutations: Cg10062 (E114N), Cg10062 (E114D), Cg10062 (E114Q), Cg10062 (H28A), Cg10062 (R70A), Cg10062 (R70K), Cg10062 (R73A), Cg10062 (R73K), Cg10062 (Y103A), Cg10062 (Y103F), Cg10062 (E114A), Cg10062 (E114D-Y103F). In a particular embodiment, the variant of Cg10062 has the Cg10062 (E114N) mutation.

For the redox reaction, one or more oxidoreductases such as YdfG, PTDH, MmsB, and SH, may be utilized, wherein the oxidoreductases may have at least 85%, at least 90% at least 95%, 96%, 97%, 98%, 99% or 100% sequence identity to SEQ ID NO: 15, 17, 18, 35, 37, 38, 55, 57, 58, 73, 75, and/or 76. In some embodiments, the redox reaction may be carried out by one oxidoreductase and may not cycle a cofactor. In a particular embodiment, the oxidoreductase may be YdfG and may have at least 85% sequence identity to SEQ ID NO: 17, 37, 57, and/or 75. In some embodiments, the one or more oxidoreductases may cycle a cofactor in pairs, such as YdfG and PTDH, or MmsB and SH.

3-HP or an anion or salt thereof may be produced as a result of a two-step reaction involving an ACA-hydrating enzyme and one or more oxidoreductases. For example, the first step may comprise hydrating ACA or an anion or salt thereof via an ACA-hydrating enzyme to generate MSA or an anion or salt thereof, and the second step may comprise converting MSA or an anion or salt thereof to 3-HP or an anion or salt thereof via an oxidoreductase. The two-step reaction may take place in vivo or in vitro. In some embodiments, one step may be performed in vivo while the other step may be performed in vitro. For example, ACA may be hydrated by an ACA-hydrating enzyme in an in vitro composition to produce MSA. In the described methods, the reaction product comprising MSA or an anion or salt thereof may comprise about 95% or more MSA or an anion or salt thereof and about 5% or less of other reaction products. The MSA reaction product may also be substantially free of acetaldehyde and CO2. The MSA from the in vitro reaction may react with an oxidoreductase expressed via a microorganism to produce 3-HP or an anion or salt thereof in vivo. In yet another embodiment, all the enzymes (ACA-hydrating enzyme and one or more oxidoreductases) may be produced in vivo, isolated from the recombinant microbe, then added to a composition where the reaction takes place in vitro.

### In Vitro

In some embodiments, MSA or an anion or salt thereof and/or 3-HP or an anion or salt thereof may be produced in vitro. For example, ACA or an anion or salt thereof and an ACA-hydrating enzyme or variant thereof as well as one or more oxidoreductase enzyme(s) may be placed in a reaction composition together, wherein 3-HP or an anion or salt thereof is prepared in vitro by a two-step reaction. Alternatively, ACA or an anion or salt thereof and an ACA-hydrating enzyme may be placed in a composition together to generate a reaction product including MSA or an anion or salt thereof. The MSA generated in vitro may then be used in another in vitro reaction wherein the MSA is added to a composition comprising one or more oxidoreductase enzymes. Alternatively, the MSA produced in vitro may be used in an in vivo reaction wherein the one or more oxidoreductase enzymes are encoded by a microorganism.

In one embodiment, a method is provided herein comprising a composition comprising an ACA-hydrating enzyme or variant thereof having at least 85% sequence identity to SEQ ID NO: 1, 4, 14, 21, 24, 34, 41, 44, 54, 59, 62, and/or 72 and/or one or more oxidoreductase enzymes having at least 85% sequence identity to SEQ ID NO: 15, 17, 18, 35, 37, 38, 55, 57, 58, 73, 75, and/or 76.

In general, 3-HP or an anion or salt thereof may be prepared via a two-step reaction in a composition as described herein. The reaction(s) may be carried out under appropriate conditions to generate MSA and/or 3-HP. Alternatively, MSA may be produced via one reaction composition and 3-HP may be produced via another. For instance, MSA from the first in vitro reaction may be used in a second in vitro reaction to generate 3-HP in a different reaction composition.

Prior to the hydrating and redox steps described herein, ACA may be synthesized by dehydrodimerization of CH4 to produce acetylene and reacting the acetylene with CO2 to produce ACA or an anion or salt thereof. The synthesized ACA or an anion or salt thereof may then be used for the methods described herein.

### In Vivo

A recombinant microbe described herein may be used to produce MSA or an anion or salt thereof and/or 3-HP or an anion or salt thereof in vivo. A method of producing 3-HP or an anion or salt thereof may include adding ACA or an anion or salt thereof to a cell culture including a recombinant microorganism and a carbon source. In some embodiments, 0.5-100 mM, such as 50 mM, ACA may be added to a cell culture at a pH of 6.6 to 8.5.

The recombinant microorganism may be genetically engineered to express an ACA-hydrating enzyme and one or more oxidoreductase enzymes. Thus, in example embodiments, a method is provided herein comprising culturing a recombinant microbe comprising an ACA-hydrating enzyme or variant thereof having at least 85% sequence identity to SEQ ID NO: 1, 4, 14, 21, 24, 34, 41, 44, 54, 59, 62, and/or 72, and/or one or more oxidoreductase enzymes having at least 85% sequence identity to SEQ ID NO: 15, 17, 18, 35, 37, 38, 55, 57, 58, 73, 75, and/or 76 in or on a suitable carbon source. These enzymes may be native or heterologous, endogenous or exogenous to the recombinant microbe.

In general, MSA and/or 3-HP may be prepared by growing and/or fermenting the recombinant microbe on or in a suitable carbon source. The recombinant microbes are grown and/or fermented under appropriate conditions for a sufficient period of time to produce MSA and/or 3-HP. In some embodiments, the cell culture containing the recombinant microbe(s) may be grown until a specific OD₆₀₀. In some embodiments, the OD₆₀₀ may be 0.3-0.9. In some embodiments, once a certain OD₆₀₀ is met, IPTG may be added to the cell culture. In some embodiments, once a certain OD₆₀₀ is met, the culture may be induced by the addition of at least 50 mM, at least 75 mM, at least 100 mM, or at least 150 mM IPTG. In a particular embodiment, at an OD₆₀₀ of 0.5, the culture may be induced by the addition of IPTG (100 mM) to a final concentration of 1 mM IPTG.

The carbon source may be culture media that comprises carbohydrates (e.g., monosaccharides, oligosaccharides, and polysaccharides), supplements (e.g., amino acids, antibiotics, polymers, acids, alcohols, aldehydes, ketones, peptides, and gases), and mineral salts. In a particular embodiment the carbon source is LB media or nitrogen (N)-mineral media with glucose as a carbon source. In a further embodiment, the method further comprises isolating MSA and/or 3-HP.

Thus, also provided herein is a cell culture comprising the recombinant microbe described herein and ACA, MSA and/or 3-HP (and anions or salts thereof).

In a further embodiment, the MSA and/or 3-HP (whether produced in vitro or in vivo) is purified. In a still further embodiment, the MSA and/or 3-HP is purified by a method such as a two-step centrifugation and water-washing; decanting centrifugation and solvent extraction from a biomass; and whole broth extraction with a water immiscible solvent. The MSA and/or 3-HP may be purified separately.

Purification and/or extraction of 3-HP has been previously described by Tengler, et al., Purification of 3-Hydroxypropionic Acid from Crude Cell Broth and Production of Acrylamide. 2013192450:A1, Dec. 27, 2013; Chemarin et al., New Insights in Reactive Extraction Mechanisms of Organic Acids: An Experimental Approach for 3-Hydroxypropionic Acid Extraction with Tri-N-Octylamine. Sep. Purif Technol. 2017, 179, 523-532; Sanchez-Castafieda et al., Organic Phase Screening for In-stream Reactive Extraction of Bio-based 3-hydroxypropionic Acid: Biocompatibility and Extraction Performances. J. Chem. Technol. Biotechnol. 2019, No. jctb.6284. doi.org/10.1002/jctb.6284; Moussa et al., Reactive Extraction of 3-hydroxypropionic Acid from Model Aqueous Solutions and Real Bioconversion Media. Comparison with Its Isomer 2-hydroxypropionic (lactic) acid, Journal of Chemical 2016; and Wasewar, K. L. Reactive Extraction: An Intensifying Approach for Carboxylic Acid Separation. IJCEA 2012, 249-255, which are each incorporated herein by reference in their entirety.

In some embodiments, the MSA and/or 3-HP may be purified to a purity of at least about 60% free (e.g., at least about 65% free, at least about 70% free, at least about 75% free, at least about 80% free, at least about 85% free, at least about 90% free, at least about 95% free, at least about 96% free, at least about 97% free, at least about 98% free, at least about 99% free) from other components with which they are associated.

### VIII. Uses

The recombinant microbes and/or reaction compositions described herein may be used for a variety of purposes. In particular, a recombinant microbe(s) or a reaction composition(s) may be used to produce MSA or an anion or salt thereof and/or 3-HP or an anion or salt thereof.

In some embodiments, the MSA and/or 3-HP prepared by a cultured recombinant microbe may be used in a composition. In some embodiments, the MSA and/or 3-HP is a reaction product produced by a recombinant microbe. In some embodiments, the MSA and/or 3-HP prepared by a reaction composition is used in a different composition to generate another product. In some embodiments, the MSA and/or 3-HP is a reaction product produced by a composition.

In some embodiments, the MSA and/or 3-HP is prepared at a time and/or location that is different than when the composition is prepared. For example, the MSA and/or 3-HP may be produced by a recombinant microbe or reaction composition in one location (e.g., a first facility, city, state, or country), transported to another location (e.g., a second facility, city, state, or country) and then incorporated into the a composition comprising a recombinant microbe or another reaction composition.

In another embodiment, the MSA or an anion or salt thereof and/or 3-HP or an anion or salt thereof prepared in vitro or in vivo may be incorporated into a product, optionally following purification. This product may be generated by combining, mixing, or otherwise using the MSA and/or 3-HP produced by the recombinant microbe or reaction composition in combination with other or more additional components to prepare the product.

Embodiments of the present technology are further illustrated through the following non-limiting examples.

### EXAMPLES

### Materials

Chemicals, biochemicals, Luria-Bertani (LB) media components and buffer salts were purchased from MilliporeSigma (Burlington, MA), Becton, Dickinson and Company (Sparks, MD), Fisher Scientific (Pittsburgh, PA) and Gold Biotechnology (St. Louis, MO). Alcohol dehydrogenase from Saccharomyces cerevisiae was purchased from Sigma Aldrich. Bradford Reagent, Precision Plus Protein standard and MINI-PROTEAN TGX Precast 4-20% polyacrylamide gels were purchased from Bio-Rad (Hercules, CA). Q5 site-directed mutagenesis kits, Monarch PCR and DNA Cleanup Kit and all restriction enzymes were purchased from New England Biolabs (Ipswich, MA). QIAprep Spin Miniprep and Maxiprep kits were purchased from Qiagen (Venlo, Netherlands). HisTrap FF 1 mL and 5 mL pre-packaged columns were purchased from Cytiva (Marlborough, MA). Amicon Ultra-15 10 K centrifugal filter units and 0.4 μM syringe filters were purchased from MilliporeSigma. Whatman Mini Uniprep G2 glass vials with glass microfiber (GMF) syringeless filters were purchased from Cytiva. Oligonucleotides were purchased from Integrated DNA Technologies (Coralville, IA). Commercially synthesized plasmids were obtained from Genscript (Piscataway, NJ). The plasmid pET-15b 12× (#61699) encoding an engineered phosphite dehydrogenase (PTDH) from Pseudomonas stutzeri was pET15b-12x was a gift from Huimin Zhao (Addgene plasmid #61699; n2t.net/addgene:61699; RRID:Addgene_61699).

### General Methods

Ampicillin and isopropyl β-D-1-thiogalactopyranoside (IPTG) stock solutions were prepared using sterile deionized water and filtered through 0.22 μM syringe filters. Following mutagenesis, plasmids were screened by restriction digestion and subsequently confirmed by sequencing. The general components used for a double restriction digest are shown in Table 1. Samples were prepared in 0.2 mL microfuge tubes and incubated at 37° C. for 1 h prior to separation on a 0.7% agarose gel.

### Media and Solutions

Luria-Bertani (LB) media was used for all experiments, unless otherwise specified. The media was prepared with tryptone (10 g L⁻¹), yeast extract (5 g L⁻¹) and NaCl (10 g L⁻¹), autoclaved and cooled to room temperature prior to culturing. SOB was prepared using tryptone (20 g L⁻¹), yeast extract (5 g L⁻¹), NaCl (0.5 g L⁻¹), 1 M MgSO₄ (10 mL L⁻¹) and autoclaved prior to use. SOC media was prepared with the addition of 2 M MgCl₂ (5 mL L⁻¹) and 1 M glucose (20 mL L⁻¹) to cooled SOB media. M9 salts were prepared using Na₂HPO₄ (6 g L⁻¹), KH₂PO₄ (3 g L⁻¹), NH₄Cl (1 g L⁻¹) and NaCl (0.5 g L⁻¹) and autoclaved. To prepare M9 minimal media, 1 M MgSO⁴ (2 mL L⁻¹), 20% w/v glucose (20 mL L⁻¹) and 1 mg mL⁻¹ thiamine hydrochloride (1 mL L⁻¹) was added to the autoclaved M9 salts. All media in this study contained ampicillin at a final concentration of 50 ng mL⁻¹. All stocks solutions used were filtered through 0.25 μM syringe filter prior to addition into media.

TABLE 1
Components of a typical restriction digest.

| | 20 μL | Final |
|---|---|---|
| Component | reaction | Concentration |
| Plasmid (100 ng/μL) | 5 μL | 500 ng |
| 10X Buffer | 2 μL | 1X |
| Restriction enzyme 1 | 1 μL | 10 U |
| Restriction enzyme 2 | 1 μL | 10 U |
| Deionized water | 11 μL | — |

### PTDH and YdfG Examples:

### Example 1: Plasmid Construction

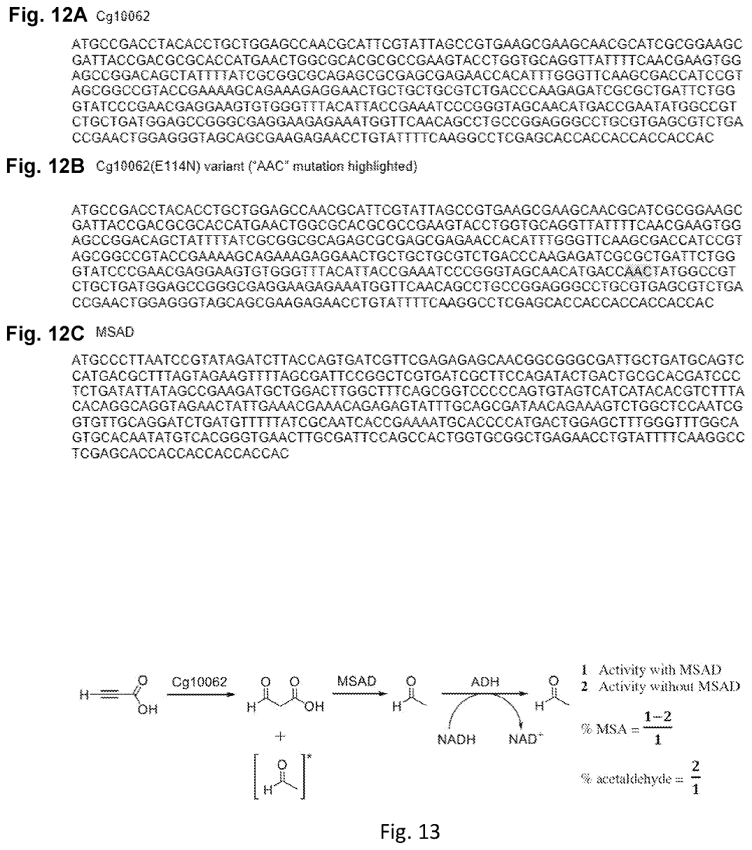

Escherichia coli strains BL21(DE3) and DH5α were obtained from Invitrogen (Carlsbad, CA). Cells were grown at 37° C. in LB media. The gene expressing Cg10062 (PDB ID: 3N4G; E.C. 3.8.1) from Corynebacterium glutamicum was codon-optimized for expression in E. coli and modified to replace the stop codon with a TEV protease recognition site (ENLYFQG) and C-terminal His6-tag (SEQ ID NO: 41) (FIG. 12A-C). The modified gene was cloned into the pET-21a(+) commercial vector, which contains a C-terminal His₆-tag, at the NdeI and XhoI restriction sites at the 5′ and 3′ positions, respectively. This plasmid was used as the parent template to engineer Cg10062 for hydratase-only activity with acetylenecarboxylate (ACA). The plasmid encoding malonate semialdehyde decarboxylase (MSAD) from Coryneform bacterium FG41 was synthesized using the same methods described above for Cg10062. The plasmid construct expressing a Hiss-tagged TEV protease (pMHTA238) was kindly provided by Professor Heedok Hong of Michigan State University. The gene encoding YdfG was amplified from E. coli W3110 genomic DNA using primers with NdeI and XhoI restriction sites at the 5′ and 3′ positions, respectively. The gene was cloned into the pET-21a(+) vector at the NdeI and XhoI sites to encode a His₆-tagged YdfG, as described above. Plasmid pET-15b 12× encoding an engineered phosphite dehydrogenase (PTDH) was used for co-factor regeneration in this study. For in vivo studies, the genes encoding Cg10062 (E114N) and YdfG were cloned into the Bgl-Brick vector pBbA1a-RFP, downstream of their own trc promoters, yielding plasmid pAS(3-HP). All plasmids and strains used in this study are listed in Table 2.

TABLE 2
Strains and plasmids used in this study.

| | | Reference |
|---|---|---|
| Strain | Genotype/Description | [Source] |
| DH5α | lacZΔM15 hsdR recA | Invitrogen |
| BL21 | fhuA2 [lon] ompT gal | NEB |
| | [dcm] ΔhsdS | |
| BL21(DE3) | fhuA2 [lon] ompT gal | NEB |
| | (λ DE3) [dcm] Δ hsdS | |

| | | Reference |
|---|---|---|
| Plasmid | Description | [Source] |
| pET-21a(+) | Ap^{R}, P_{T7}, C-terminal His₆ | Novagen |
| pBbA1a-RFP | Ap^{R}, P_{trc} | Addgene |
| pCg10062 | cg10062 in pET-21a(+) | this study |
| pYdfG | ydfg in pET-21a(+) | this study |
| pET-15b 12x | pET-15b with Opt 12x ptdh and | Addgene |
| | N-terminal His₆ | |
| pAS(3HP) | cg10062 and ydfg in pBbA1a-RFP | this study |

### Example 2: Q5 Site-directed Mutagenesis and Transformation of PCR Product

Unless otherwise indicated, the plasmid encoding wild-type Cg10062 was used as the template for Q5 site-directed mutagenesis to construct the Cg10062 variants. PCR was carried out in a Bio-Rad DNA Engine Peltier Thermal Cycler (Hercules, CA). The Q5 site-directed mutagenesis was carried out in 3 steps. Step 1 includes exponential amplification from parent template (Tables 3 and 4) using the primers listed in Table 5. Step 2 is Kinase, Ligase and DpnI (KLD) treatment (Table 6) of the resulting PCR product. The final step is the transformation of the KLD product to isolate the plasmid with the desired modification.

TABLE 3
Exponential amplification from parent template.

| | 25 μL | Final |
|---|---|---|
| Component | reaction | Concentration |
| Q5 Hot State High-Fidelity | 12.5 μL | 1X |
| 2X Master Mix | | |
| 10 μM Forward Primer | 1.25 μL | 0.5 μM |
| 10 μM Reverse Primer | 1.25 μL | 0.5 μM |
| Template DNA (5 ng/μL) | 1 μL | 5 ng |
| Deionized water | 9 μL | — |

TABLE 4
PCR conditions for Q5 site-directed mutagenesis.

| | | |
|---|---|---|
| Step | Temperature | Time |

| | | | | |
|---|---|---|---|---|
| Initial | 98° | C. | 30 | sec |
| Denaturation | 98° | C. | 10 | sec |
| 30 cycles | 50-72° | C.* | 30 | sec |
| | 72° | C. | 3 | min (30 sec/kb) |
| Final Extension | 72° | C. | 2 | min |

| | | | |
|---|---|---|---|
| Hold | 4° | C. | — |

*Annealing temperatures for each pair of primers was determined using NEBasechanger.

TABLE 5
Primers used for the construction of Cg10062
mutants. Codons used to introduce mutations
are underlined and bold.

| Enzyme | | |
|---|---|---|
| Variant | Primer | Sequence |
| Cg10062 | AS001 | CAACATGACCCAGTATGGCCGTC |
| (E114Q) | (F) | (SEQ ID NO: 77) |
| | AS002 | CTACCCGGGATTTCGGTA |
| | (R) | (SEQ ID NO: 78) |
| Cg10062 | AS003 | CAACATGACCGATTATGGCCGTC |
| (E114D) | (F) | (SEQ ID NO: 79) |
| | AS002 | CTACCCGGGATTTCGGTA |
| | (R) | (SEQ ID NO: 78) |
| Cg10062 | AS012 | TACCGACGCGGCGCATGAACTGGCGCACG |
| (H28A) | (F) | (SEQ ID NO: 80) |
| | AS013 | ATCGCTTCCGCGATGCGT |
| | (R) | (SEQ ID NO: 81) |
| Cg10062 | AS014 | AGCGACCATCGCGAGCGGCCGTAC |
| (R70A) | (F) | (SEQ ID NO: 82) |
| | AS015 | TGAACCCAAATGTGGTTC |
| | (R) | (SEQ ID NO: 83) |
| Cg10062 | AS016 | AGCGACCATCAAAAGCGGCCGTA |
| (R70K) | (F) | (SEQ ID NO: 84) |
| | AS017 | TGAACCCAAATGTGGTTCTC |
| | (R) | (SEQ ID NO: 85) |
| Cg10062 | AS018 | CCGTAGCGGCGCGACCGAAAAGC |
| (R73A) | (F) | (SEQ ID NO: 86) |
| | AS019 | ATGGTCGCTTGAACCCAA |
| | (R) | (SEQ ID NO: 87) |
| Cg10062 | AS020 | CCGTAGCGGCAAAACCGAAAAGC |
| (R73K) | (F) | (SEQ ID NO: 88) |
| | AS019 | ATGGTCGCTTGAACCCAA |
| | (R) | (SEQ ID NO: 87) |
| Cg10062 | AS021 | AGTGTGGGTTGCGATTACCGAAATCCCGGG |
| (Y103A) | (F) | (SEQ ID NO: 89) |
| | AS022 | TCCTCGTTCGGGATACCC |
| | (R) | (SEQ ID NO: 90) |
| Cg10062 | AS023 | AGTGTGGGTTTTTATTACCGAAATCCC |
| (Y103F) | (F) | (SEQ ID NO: 91) |
| | AS022 | TCCTCGTTCGGGATACCC |
| | (R) | (SEQ ID NO: 90) |
| Cg10062 | AS024 | CAACATGACCGCGTATGGCCGTCTG |
| (E114A) | (F) | (SEQ ID NO: 92) |
| | AS002 | CTACCCGGGATTTCGGTA |
| | (R) | (SEQ ID NO: 78) |
| Cg10062 | AS026 | CAACATGACCAACTATGGCCGTCTG |
| (E114N) | (F) | (SEQ ID NO: 93) |
| | AS002 | CTACCCGGGATTTCGGTA |
| | (R) | (SEQ ID NO: 78) |
| *Cg10062 | AS023 | AGTGTGGGTTTTTATTACCGAAATCCC |
| (E114D- | (F) | (SEQ ID NO: 91) |
| Y103F) | AS002 | CTACCCGGGATTTCGGTA |
| | (R) | (SEQ ID NO: 78) |

*Plasmid expressing Cg10062(E114D) was used as a template.

TABLE 6
KLD treatment of PCR product.

| | 10 μL | Final |
|---|---|---|
| Component | reaction | Concentration |
| PCR Product | 1 μL | — |
| 2X KLD Reaction Buffer | 5 μL | 1X |
| 10X KLD Enzyme Mix | 1 μL | 1X |
| Deionized water | 3 μL | — |

Transformations were carried out using a Bio-Rad Gene Pulser II electroporation system (Hercules, CA). For the transformation of KLD product, 50 μL E. coli DH5α electrocompetent cells were thawed on ice and 5 μL of the KLD product was added to the electrocompetent cells. The sample was transferred to a cold sterile Gene Pulser electroporation cuvette and the cells were pulsed at 2.5 kV (25 μF capacitance, 200Ω resistance). The cells were carefully resuspended in 1 mL SOC and shaken at 37° C. for 1 h. The cells were pelleted at 17,000×g in a microcentrifuge and the SOC was decanted. The cells were resuspended in 100 μL SOC, spread onto LB plates and incubated at 37° C. overnight.

Multiple colonies were screened by restriction digestion to identify plasmids containing the desired mutation. Single colonies were inoculated into separate culture tubes containing 5 mL of LB and the cultures were shaken at 37° C. overnight. DNA extraction from the cell pellets was carried out using a QIAprep Miniprep or Maxiprep kit following the manufacturer instructions. Isolated DNA was sequenced at the Michigan State University Research Technology Support Facility (MSU RTSF) Genomics Core and the plasmids containing the desired mutations were transformed into electrocompetent E. coli BL21(DE3) for protein expression.

### Example 3: Protein Expression, Purification and Quantification

Each plasmid encoding a gene of interest was transformed into electrocompetent E. coli BL21(DE3). A single colony was inoculated into 25 mL LB, and the cultures were shaken overnight at 37° C. The overnight culture was used to inoculate 1 L LB (in a 4 L Erlenmeyer flask), to an initial OD₆₀₀ of 0.05, and the culture was incubated at 37° C. with shaking. When an OD₆₀₀ of 0.5-0.7 was reached, IPTG was added to a final concentration of 1 mM. The culture was then shaken at 30° C. for 8-10 h. Cells were harvested by centrifugation (4500×g, 4° C., 10 mins) and stored at −20° C.

After thawing, cells were resuspended in lysis buffer (20 mM sodium phosphate pH 7.2 and 20 mM imidazole) (2 mL lysis buffer per gram of cell paste). Cells were lysed by two passages through a French Pressure cell (Thermo Scientific, Waltham, MA) at 18,000 psi. The cellular lysate was centrifuged (47,500×g, 4° C., 10 mins) and filtered through a 0.45 μm sterile syringe filter.

All enzymes were purified on an ÄKTA Start FPLC system (Cytiva) equipped with a HisTrap FF 1 mL or 5 mL nickel affinity column. The binding buffer contained 20 mM sodium phosphate pH 7.2 and 500 mM sodium chloride. The elution buffer contained 20 mM sodium phosphate pH 7.2, 500 mM sodium chloride and 500 mM imidazole. An imidazole gradient from 20 mM to 500 mM imidazole was used to elute protein over 20 column volumes. Fractions containing the protein of interest were pooled, concentrated, and desalted using Amicon Ultra-15 10K filters. All purifications yielded 50-150 mg enzyme per liter of cell culture.

Protein concentrations of cell lysates and purified enzyme were quantified using Bradford protein assay and 6 M guanidinium chloride, respectively. For quantification of cell lysates, 4 μL of crude lysate was diluted in 16 μL of deionized water and incubated with 1 mL Bradford reagent at room temperature for 10 mins prior to OD₅₉₅ measurements. The purified protein was quantified using the molar extinction coefficient of each protein at 280 nm and the molecular weight (Table 7). To prepare samples, 10 μL of the protein sample was diluted with 990 ρL of 6 M guanidinium chloride prior to measuring the absorbance at 280 nm.

TABLE 7
Parameters used for enzyme quantification.

| Enzyme | MW (Da) | ε₂₈₀ (M⁻¹ cm⁻¹) |
|---|---|---|
| Cg10062 | 19013 | 30440 |
| Cg10062(E114Q) | 19012 | 30440 |
| Cg10062(E114D) | 18999 | 30440 |
| Cg10062(R70A) | 18928 | 29160 |
| Cg10062(R73A) | 18928 | 29160 |
| Cg10062(R70K) | 18985 | 30440 |
| Cg10062(R73K) | 18985 | 30440 |
| Cg10062(E114A) | 18955 | 29160 |
| Cg10062(H28A) | 18947 | 29160 |
| Cg10062(Y103A) | 18921 | 29160 |
| Cg10062(Y103F) | 18997 | 29160 |
| Cg10062(E114N) | 18998 | 30440 |
| YdfG | 28313 | 36130 |
| MSAD | 16464 | 8250 |
| 12x Opt PTDH | 36568 | 26600 |

### Example 4: Cg10062 Novel Variant Discovery

Cg10062 from Corynebacterium glutamicum (SEQ ID NO: 1) was identified as an enzyme belonging to the tautomerase superfamily. Enzymes belonging to this superfamily have a characteristic β-α-β fold and a catalytic N-terminal proline residue. Cg10062 is a homotrimer of 149 amino acids and its native function is unknown. However, it has the ability to accept a range of acetylenic substrates, including ACA. Wild-type Cg10062 catalyzes the hydration and subsequent hydration-dependent decarboxylation to produce a mixture of malonate semialdehyde (25%) and acetaldehyde (75%). Six residues, Pro-1, His-28, Arg-70, Arg-73, Tyr-103 and Glu-114 have been identified as catalytic residues important for Cg10062 activity. Furthermore, Cg10062 does not require metal co-factors, coenzymes, or CoA substrates, making it a highly attractive candidate for ACA hydration. Two variants Cg10062 (E114Q) (SEQ ID NO: 2) and Cg10062 (E114D) (SEQ ID NO: 3) were previously described which produce MSA exclusively from ACA hydration, but both variants display significantly lower activity relative to the Cg10062.

Using a combination of modeling, site-directed mutagenesis, kinetic characterization and X-ray crystallography, a novel variant of Cg10062 was discovered with activity comparable to the wild-type, but which produced only malonic semialdehyde from ACA hydration. Cg10062 (E114N) (SEQ ID NO: 4) is a non-decarboxylating variant of Cg10062 with hydratase-only activity and produces only malonic semialdehyde. The differences in rates from the coupled enzyme assay described in the experiments above, in the absence and presence of malonate semialdehyde decarboxylase (MSAD), was used to generate a product profile for each enzyme (Table 8). Further kinetic characterization showed that Cg10062 (E114N) had a k_{cat} 1.5-fold and 3-fold higher than the E114D and E114Q, respectively. The overall catalytic efficiency of the newly discovered hydratase-only variant was comparable to that of the wild-type enzyme (Table 9).

TABLE 8
The product profile of the Cg10062 and mutants determined
from Cg10062 activity in the presence and absence of
malonate semialdehyde decarboxylase (MSAD). (*from non-
enzymatic decarboxylation of malonate semialdehyde).

| | |
|---|---|
| Cg10062 | Product Ratio (%) |

| variant | malonate semialdehyde | acetaldehyde |
|---|---|---|
| Wild-type | 19 | 81 |
| E114Q | >99 | <1* |
| E114D | >99 | <1* |
| E114D-Y103F | >99 | <1* |
| E114N | >99 | <1* |

TABLE 9
Kinetic parameters for Cg10062 variants and
other enzymes used for 3-HP synthesis.

| Enzyme | Kₘ (μM) | k_{cat} (s⁻¹) | k_{cat}/Kₘ × 10⁴ (M⁻¹s⁻¹) |
|---|---|---|---|
| Cg10062 | 66 ± 4 | 8.21 ± 0.26 | (12.4 ± 0.8) |
| Cg10062(E114Q) | 64 ± 12 | 0.66 ± 0.04 | (1.08 ± 0.21) |
| Cg10062(E114D) | 557 ± 62 | 1.12 ± 0.05 | (0.20 ± 0.02) |
| Cg10062(E114D- | 72 ± 7 | 0.34 ± 0.01 | (0.47 ± 0.05) |
| Y103F) | | | |
| Cg10062(E114N) | 45 ± 1 | 1.04 ± 0.07 | (1.19 ± 0.03) |
| YdfG | 137 ± 18 | 6.62 ± 0.43 | (4.83 ± 0.71) |
| PTDH | 778 ± 44 | 3.45 ± 0.06 | (0.44 ± 0.03) |
| MmsB | 2220 ± 192 | 101 ± 5 | (4.55 ± 0.59) |

*Steady-state kinetics of Cg10062 and variants upon incubation with ACA were monitored using a coupled enzyme assay described elsewhere, in 100 mM sodium phosphate pH 8.0 at 25° C.

### Example 5: Kinetic Characterization of Cg10062 and Variants

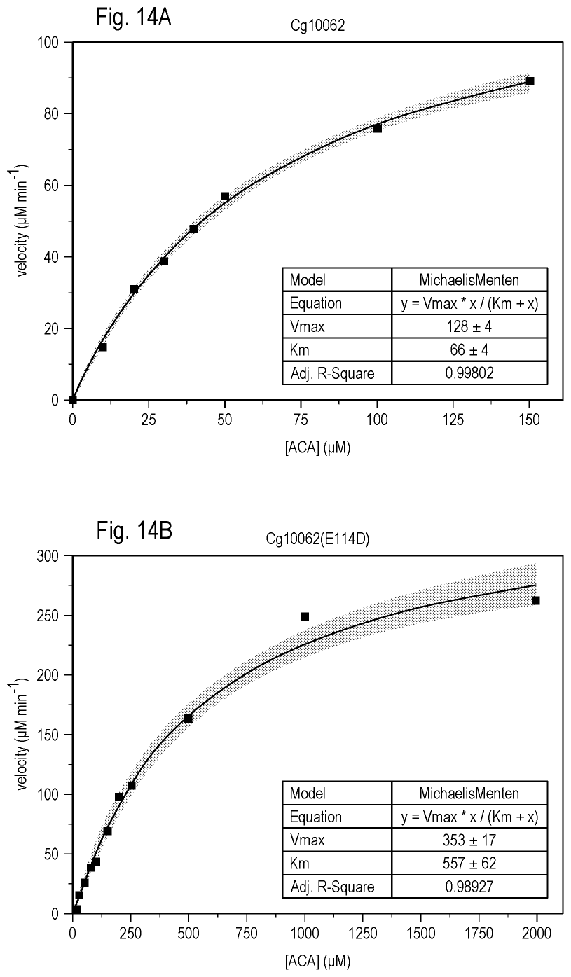

Steady-state kinetics were carried out using a Molecular Devices SpectraMax iD3 multi-mode microplate reader and Shimadzu UV2600 spectrophotometer. All assays were carried out in triplicate at 25° C. in 100 mM sodium phosphate pH 8.0 with a final volume of 200 μL, unless otherwise specified. Enzyme activity was measured using the coupled enzyme activity shown in FIG. 13. The reduction of acetaldehyde by NADH-dependent alcohol dehydrogenase (ADH) was monitored by following the oxidation of NADH at 340 nm (ε=6220 M⁻¹ cm⁻¹).

All stock solutions, except ADH, required for the kinetics assays used for determining hydratase and hydratase/decarboxylase activities were prepared in 100 mM sodium phosphate pH 8.0. The ACA stock solution was prepared by diluting the appropriate volume of ACA in sterile 100 mM sodium phosphate pH 8.0 and adjusting the pH back to 8.0 using 10 N sodium hydroxide. Stock solutions of ADH were prepared using deionized water, as recommended by the manufacturer. Initial screening assays contained NADH (0.3 mM, 10 μL of a 5 mg mL⁻¹ stock), ADH (12 U), MSAD (1.2 U), ACA pH 8 (0.5 mM, 20 μL of a 5 mM stock) and Cg10062 or variant (0.025-0.5 mg mL⁻¹). The final pH of each assay was 8.

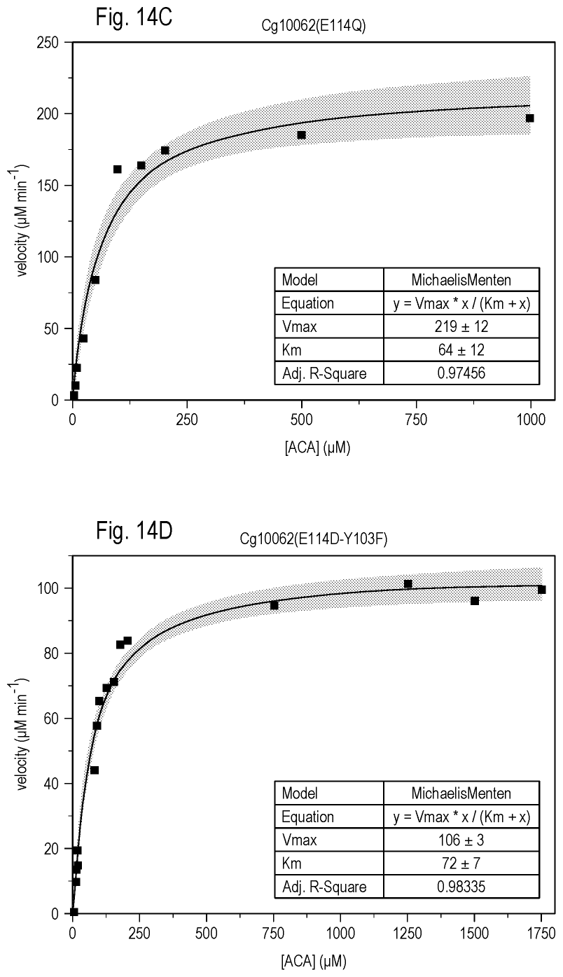

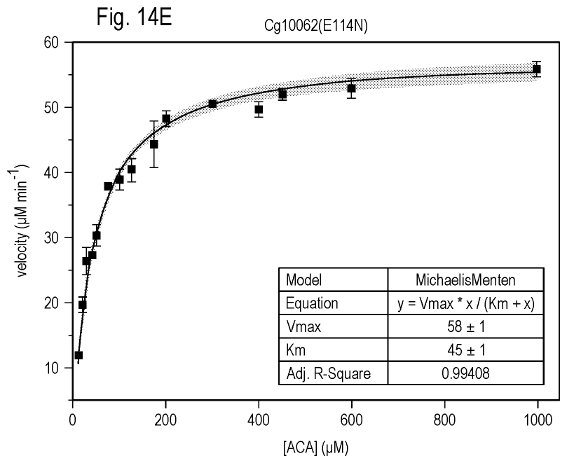

The amount of enzyme used in each assay was varied in order to observe measurable activity. Thus, the rates obtained from this experiment were not used directly to compare the enzyme activity. These activities were only used for establishing the product profile of each enzyme. The ratios of MSA and acetaldehyde formed by each enzyme was determined by the coupled enzyme assay (FIG. 13), using the differences in rates in the presence and absence of MSAD (Table 10). Variants that showed hydratase-only activity, indicated by the lack of absorbance change in the absence of MSAD, were further characterized to include measurement of kinetic parameters. The initial rates of these non-decarboxylating Cg10062 mutants relative to varied ACA concentrations (1-5000 μM) were plotted to fit the Michaelis-Menten model and analyzed using Origin 9.0 (FIG. 14A-E). All other components used for steady-state kinetics were identical to those used in the initial screening assays.

TABLE 10
Rates of Cg10062 and variants in
the presence and absence of MSAD.

| | | Rate in the | Rate in the |
|---|---|---|---|
| | Final | presence | absence |
| Cg10062 | concentration | of MSAD | of MSAD |
| variant | (mg mL⁻¹) | (μM min⁻¹) | (μM min⁻¹) |
| Wild-type | 0.025 | 146 | 120 |
| E114N | 0.025 | 67 | no activity |
| E114Q | 0.10 | 102 | no activity |
| E114D | 0.10 | 90 | no activity |
| E114D-Y103F | 0.10 | 39 | no activity |
| H28A | 0.25 | 12 | 4.5 |
| E114A | 1.50 | 15 | 11 |
| Y103A | 0.25 | 38 | 16 |
| Y103F | 0.15 | 65 | 43 |
| R70A | 0.25 | no activity | no activity |
| R70K | 0.25 | no activity | no activity |
| R73A | 0.25 | no activity | no activity |
| R73A | 0.75 | no activity | no activity |

### Example 6: ¹H NMR Characterization of Cg10062-catalyzed Hydration of ACA

Identification of products from wild-type Cg10062 and mutant-catalyzed reactions with ACA was determined by ¹H NMR spectroscopy on a 500 MHz Varian NMR spectrophotometer and analyzed using MestReNova. (H)wet1D was used for solvent suppression of the large HOD peak since all assays were carried out in 100 mM sodium phosphate pH 8.0. DMSO-d₆ (δ 2.49) was used as a lock signal and TSP (3-(trimethylsilyl) propionate-2,2,3,3-d₄ sodium salt) (δ −0.21 (s, 9H)) was used as an internal standard.

All stock solutions were prepared in 100 mM sodium phosphate pH 8.0. To prepare a 1 M stock solution of ACA pH 8, an appropriate volume of ACA was diluted in 100 mM sodium phosphate pH 8.0 and neutralized with 10 N sodium hydroxide, in a volumetric flask. ACA (111 mM, 20 μL of 5 M stock) was added in 830 μL of 100 mM sodium phosphate pH 8.0. A reaction was initiated by the addition of Cg10062 or variant (50 μL of 4.8 mg mL⁻¹). Reactions were incubated at 25° C. To examine reaction progress, aliquots (150 μL) were removed and quenched with 2 μL 5 M H₂SO₄. One sample was removed immediately following reaction initiation (t=0 h) and a second sample was quenched after 1 h. The samples were centrifuged (17,000×g, 5 mins) to remove precipitated protein. Each sample (100 μL) was combined with TSP (10 mM, 70 μL of a 100 mM stock), and DMSO-d₆ (30 μL). The final volume was adjusted to 700 μL using 100 mM sodium phosphate pH 8.0 for NMR spectroscopy.

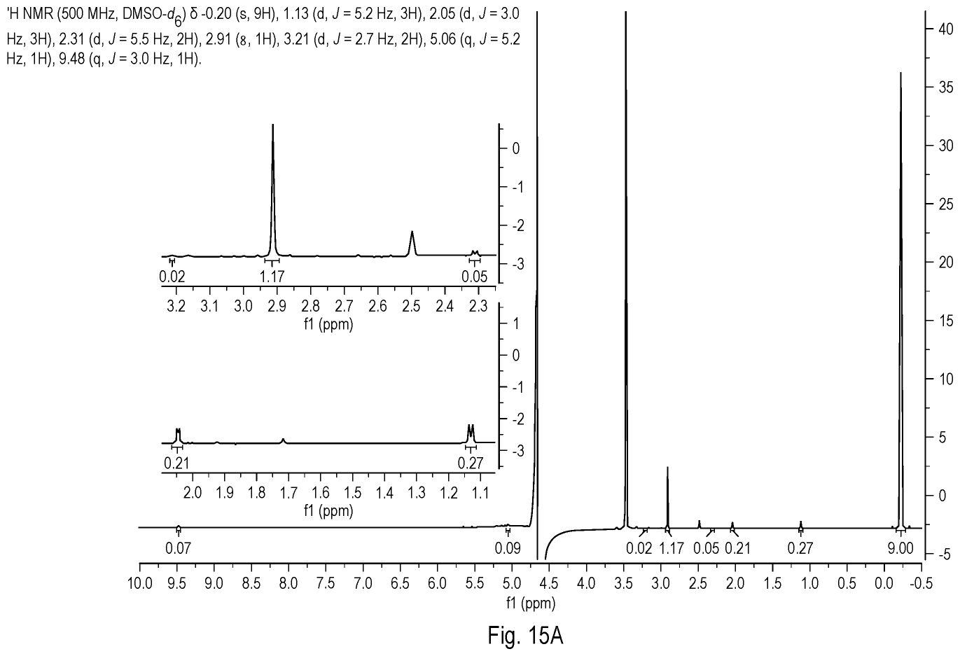

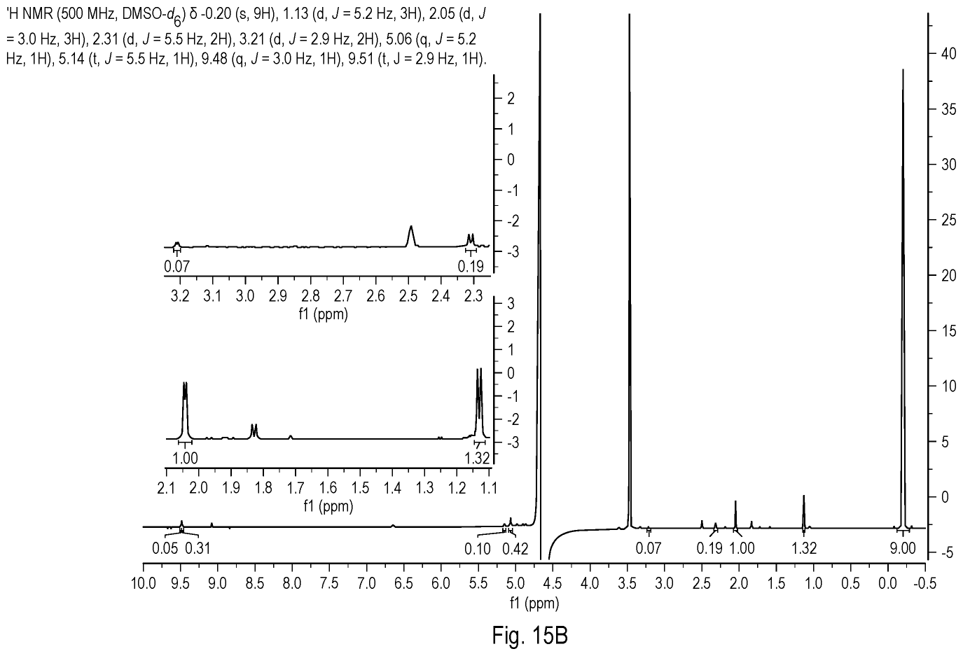

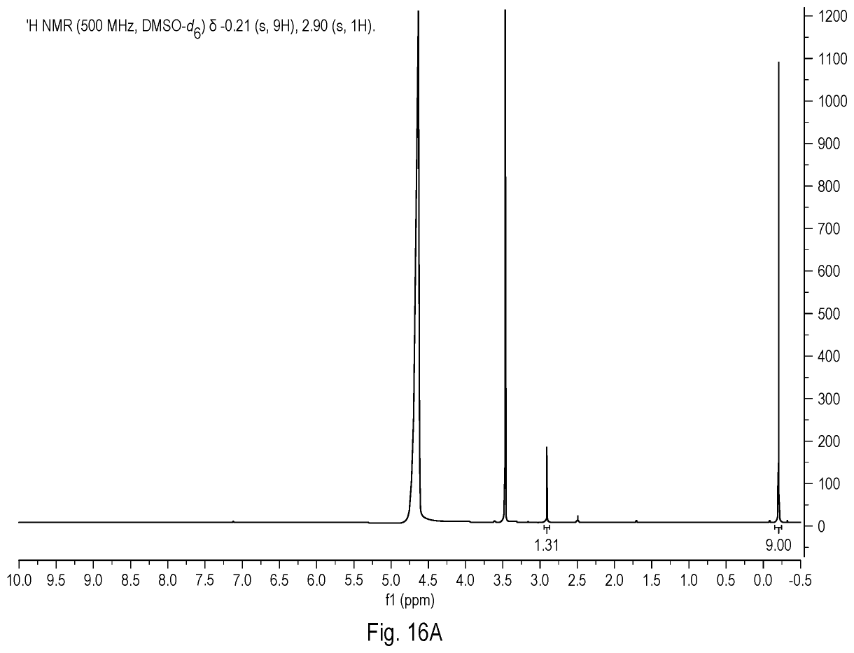

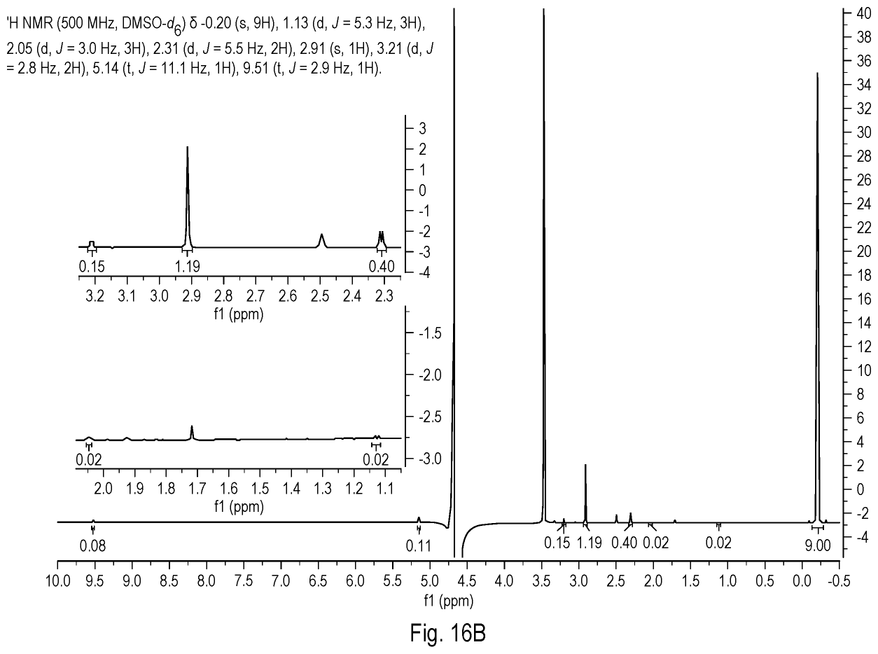

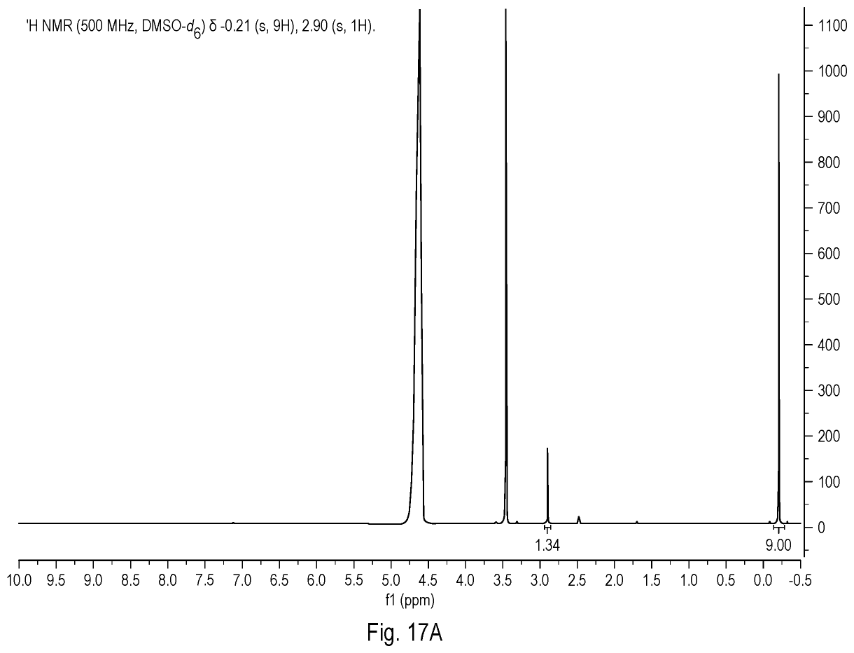

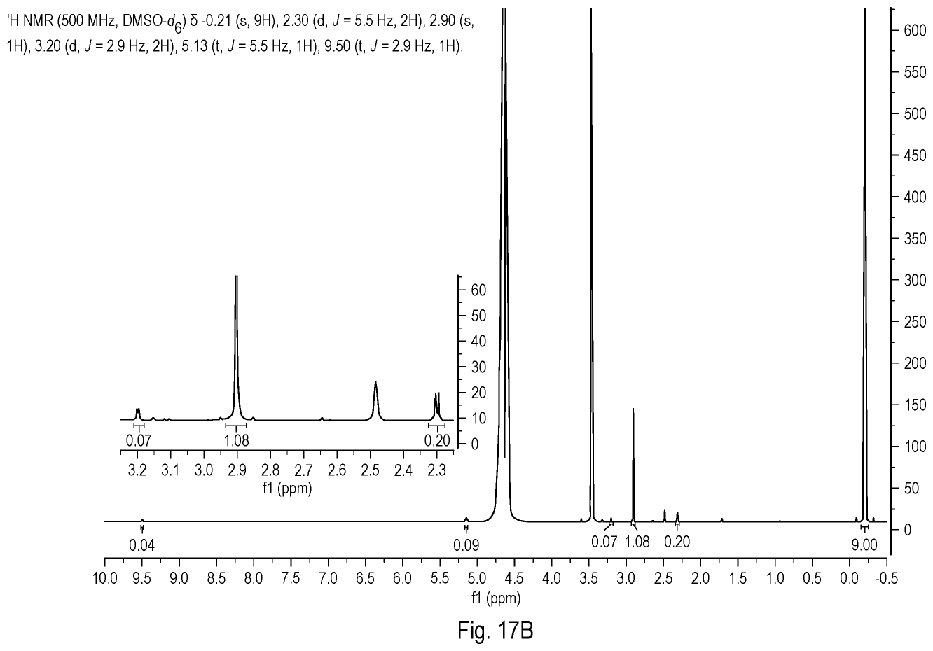

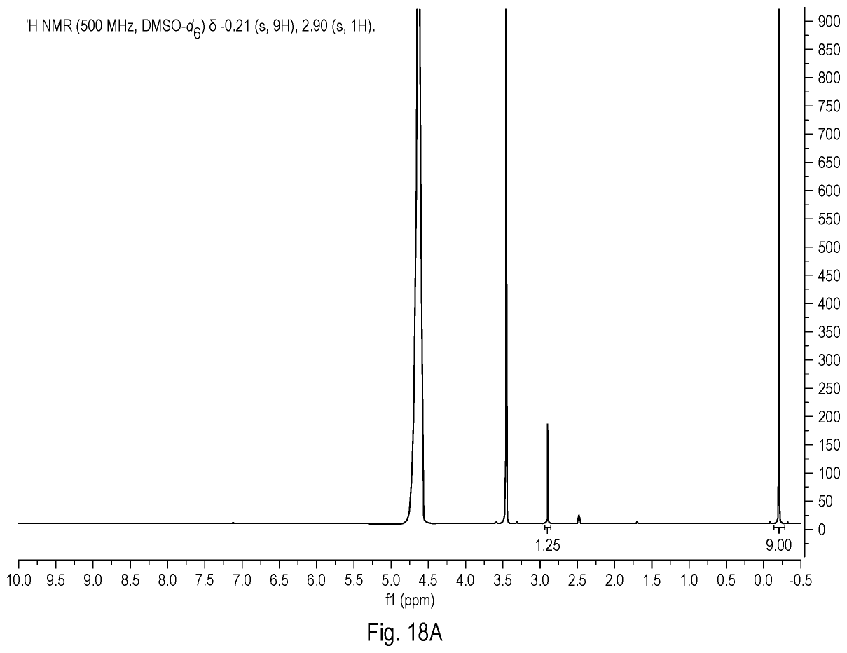

¹H NMR spectra were obtained for each sample (64 scans; 10 s) (FIGS. 15A-B, 16A-B, 17A-B and 18A-B). The resonance at δ 2.91; (s, 1H) corresponds to ACA. Resonances at δ 3.20; (d, 2H), δ 9.50; (t, 1H) and δ 2.30; (d, 2H), 5.13; (t, 1H) correspond to malonate semialdehyde and its hydrate, respectively. Resonances at δ 2.03; (d, 3H), 9.47; (q, 1H) and δ 1.12; (d, 3H), 5.05; (q, 1H) correspond to acetaldehyde and its hydrate, respectively.

### Example 7: Kinetic Characterization of YdfG

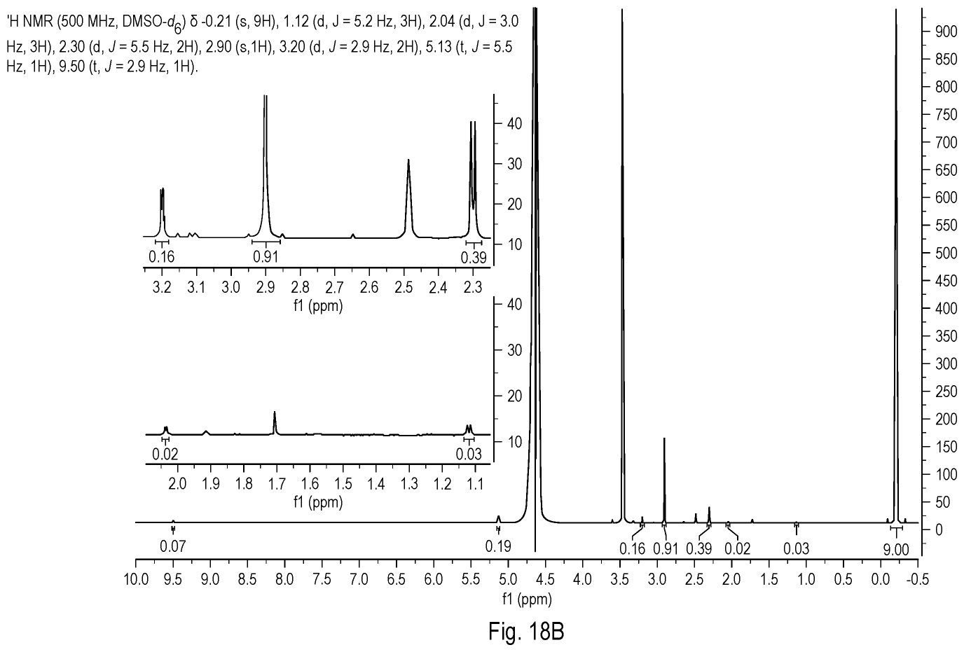

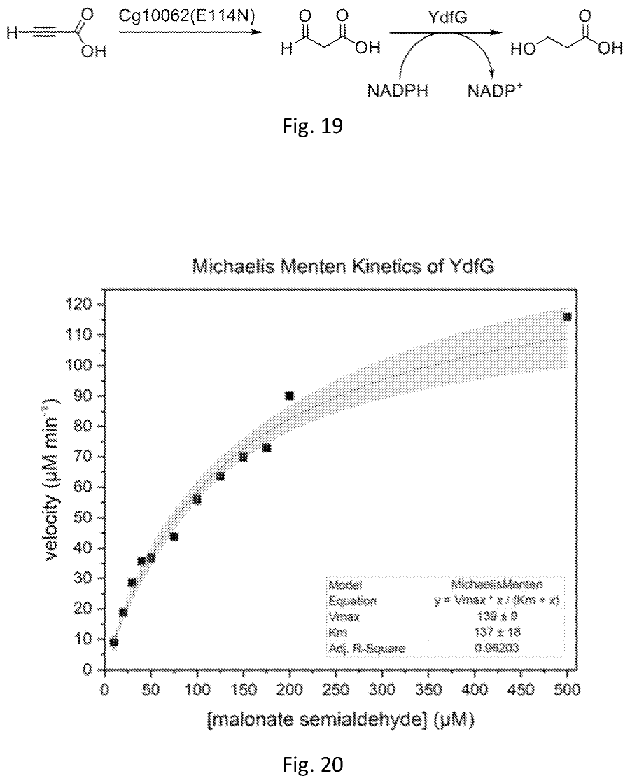

YdfG was characterized using the coupled enzyme assay show in FIG. 19. All assays were carried out in triplicate at 25° C. in 100 mM sodium phosphate pH 8.0, in a final volume of 200 μL, unless otherwise specified. All stock solutions prepared for the assays were prepared in 100 mM sodium phosphate pH 8.0. The specific activity of YdfG was measured by generating MSA in situ from the Cg10062 (E114N)-catalyzed hydration of ACA. The assay contained a large excess of Cg10062 (E114N) (2 U), YdfG (0.005 mg mL⁻¹, 10 μL of a 0.1 mg mL⁻¹ stock) and NADPH (0.3 mM, 10 μL of a 5 mg mL⁻¹ stock). The assays were initiated with the addition of ACA (10-2000 μM). See FIG. 20.

### Example 8: ¹H NMR Characterization of YdfG-catalyzed Reduction of MSA

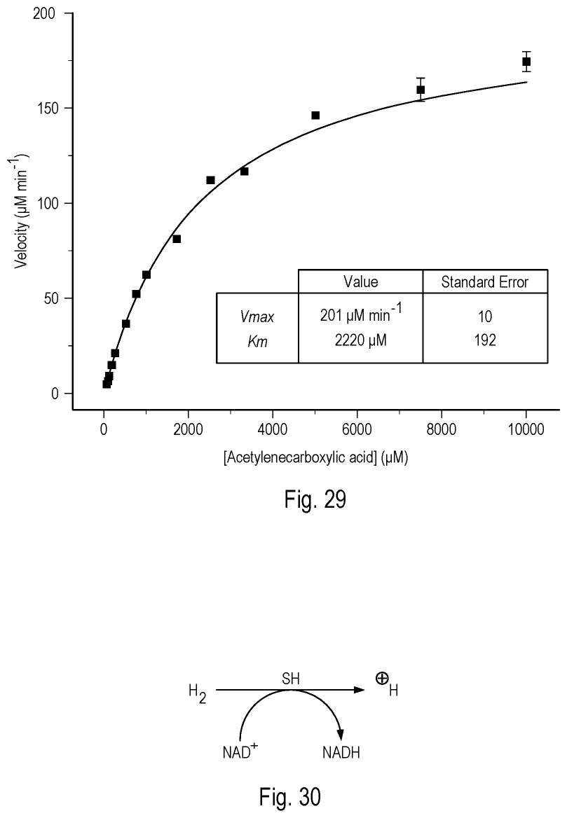

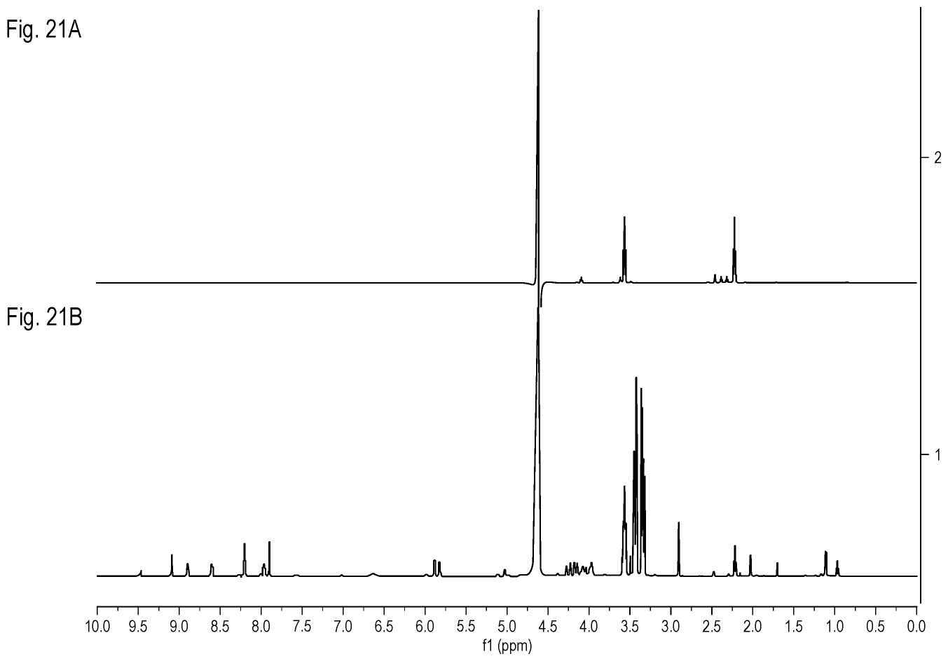

Cg10062 (E114D)-catalyzed hydration of ACA was used to produce MSA in situ. ACA (20 mM, 14 μL of 1 M stock) was combined with YdfG (20 μL of a 6 mg mL⁻¹ stock), TSP (10 mM, 70 μL of a 100 mM stock), and DMSO-d₆ (30 μL). The volume was adjusted to 680 μL with 100 mM sodium phosphate pH 8.0. The reaction was initiated with the addition of Cg10062 (E114D) (20 μL of 3 mg mL⁻¹ stock). ¹H NMR spectra were obtained after incubating the samples at 25° C. for 1 h. The resonance at δ 2.91; (s, 1H) corresponds to ACA. Resonances at δ 2.23; (t, 2H) and δ 3.58; (t, 2H) correspond to 3-hydroxypropionate. See FIG. 21A-B.

### Example 9: Kinetic Characterization of PTDH

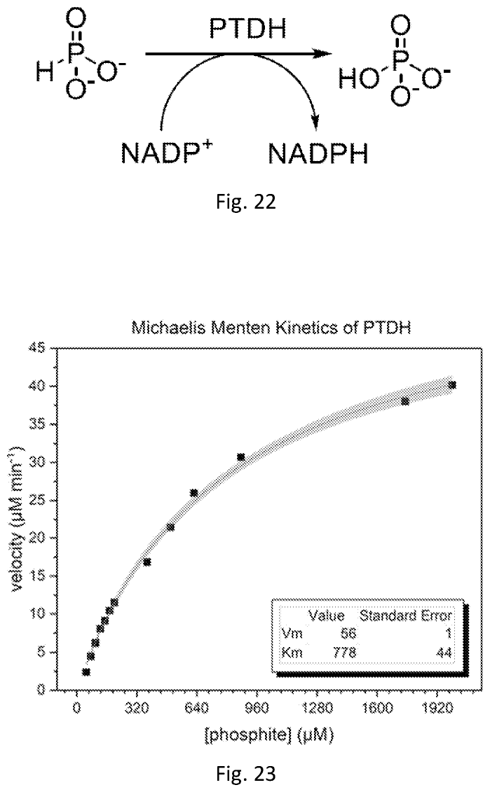

PTDH activity was measured using the assay shown in FIG. 22. All assays were carried out in triplicate at 25° C. in 100 mM sodium phosphate pH 8.0 in a final volume of 200 μL, unless otherwise specified. All stock solutions were prepared in 100 mM sodium phosphate pH 8.0, unless otherwise specified. A sodium phosphite stock solution was prepared by dissolving an appropriate amount of the solid in a volumetric flask with water.

The assay contained PTDH (0.05 mg mL⁻¹, 10 μL of a 1 mg mL⁻¹ stock) and NADP⁺ (0.3 mM, 10 μL of a 5 mg mL⁻¹ stock). The assays were initiated with the addition of sodium phosphite in varying concentrations (10-1000 μM). See. FIG. 23.

### Example 10: pH Dependence of Cg10062 (E114N), YdfG and PTDH

The pH dependence of each enzyme was measured using four different buffer systems: 100 mM citrate-phosphate, 100 mM sodium phosphate, 50 mM bis tris propane (BTP) and 100 mM sodium carbonate/bicarbonate buffers for pH 3.6-5.6, 6.0-8.0, 7.6-9.2 and 9.2-9.6, respectively. The pH dependence of each enzyme was studied using the respective enzyme assay used for kinetic characterization as described previously. All pH studies were carried out in triplicate (1 mL) on a Shimadzu UV2600 spectrophotometer at 25° C. to ensure that the final pH of each assay remained unchanged with the addition of assay components. All stock solutions were prepared in 100 mM sodium phosphate pH 8.0, unless otherwise specified and the assays were carried out in the respective buffers for each pH. For each assay, all components except the substrate were combined and prepared in 1 mL microfuge tubes and incubated at 25° C. for 30 mins.

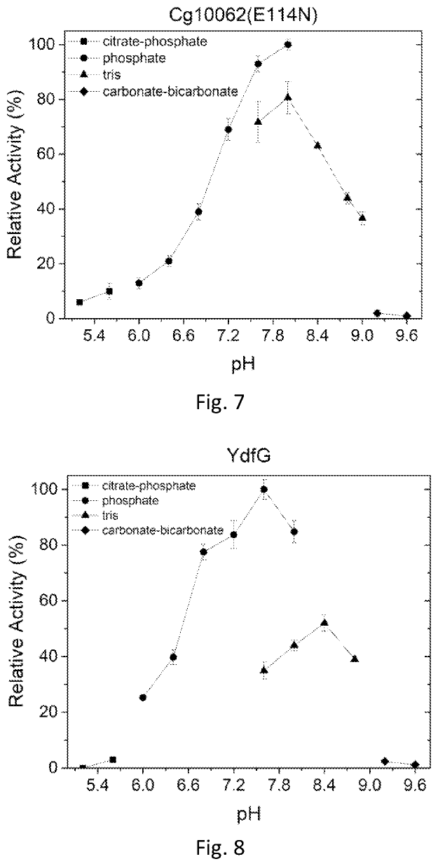

Cg10062 (E1141V) pH dependence assay: Cg10062 (E114N) (0.05 U, 10 μL of a 1 mg mL⁻¹ stock), MSAD (1.2 U), ADH (12 U) and NADH (1.2 mM, 10 μL of a 20 mg mL⁻¹ stock) was combined with 920 μL of the prepared buffers. The assays were initiated by the addition of ACA (1 mM, 10 μL of a 100 mM stock). See, FIG. 7.

YdfG pH dependence assay: YdfG (0.2 U, 10 μL of a 1 mg mL⁻¹ stock), Cg10062 (E114N) (1.5 U) and NADPH (1.2 mM, 10 μL of a 20 mg mL⁻¹ stock) was combined with 960 μL of the prepared buffers. The assays were initiated by the addition of ACA (1 mM, 10 μL of a 100 mM stock). See, FIG. 8.

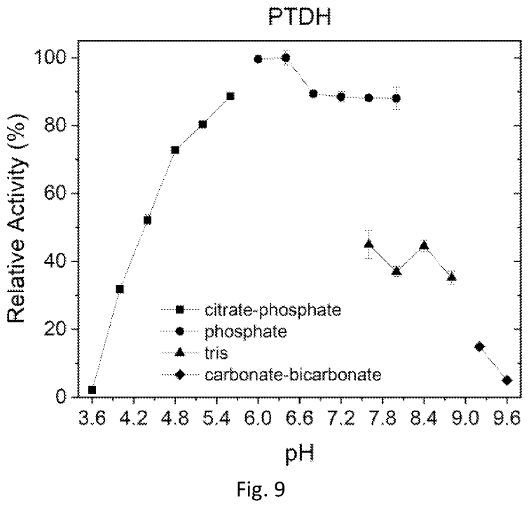

PTDH pH dependence assay. PTDH (0.05 U, 10 μL of a 5 mg mL⁻¹ stock) and NADP⁺ (1.2 mM, 10 μL of a 20 mg mL⁻¹ stock) was combined with 970 μL of the prepared buffers. The assays were initiated by the addition of sodium phosphite (10 mM, 10 μL of a 1 M stock). See, FIG. 9.

Upon testing the pH dependence of the three enzymes involved in this biocatalytic route to 3-HP, it was determined that a system maintained at pH 8.0 would provide optimal activity for the efficient conversion of ACA to 3-HP (FIG. 7-9).

### Example 11: 3-HP Synthesis in vitro with Cofactor Recycling

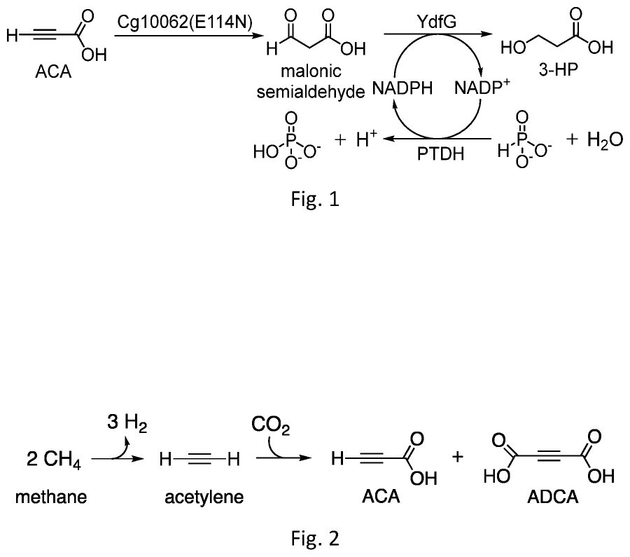

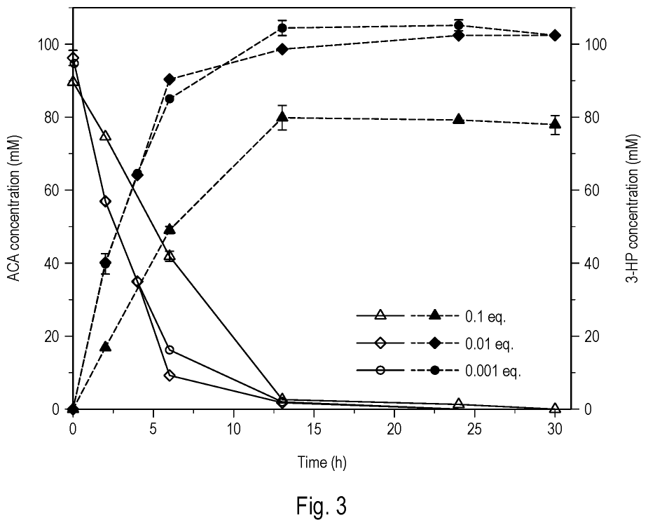

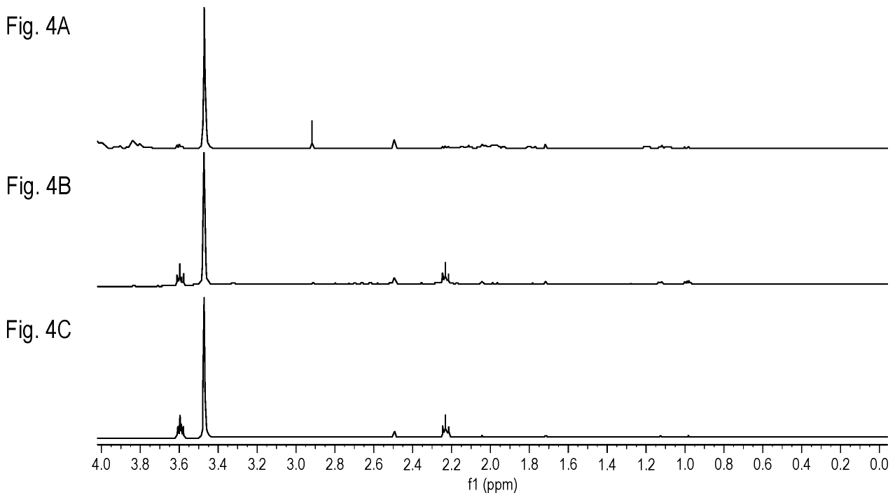

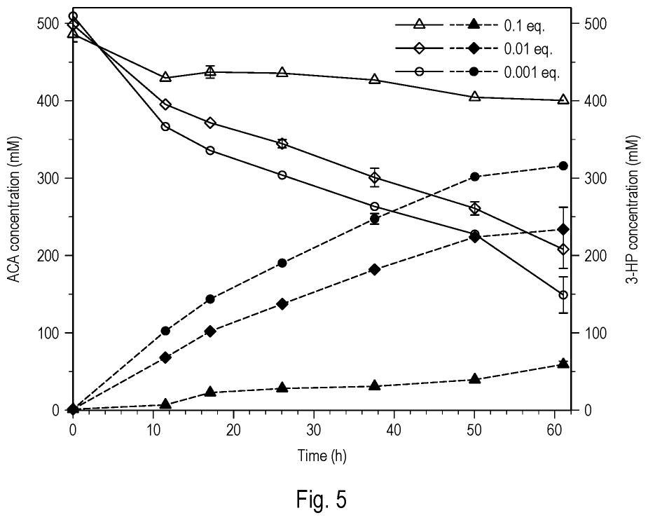

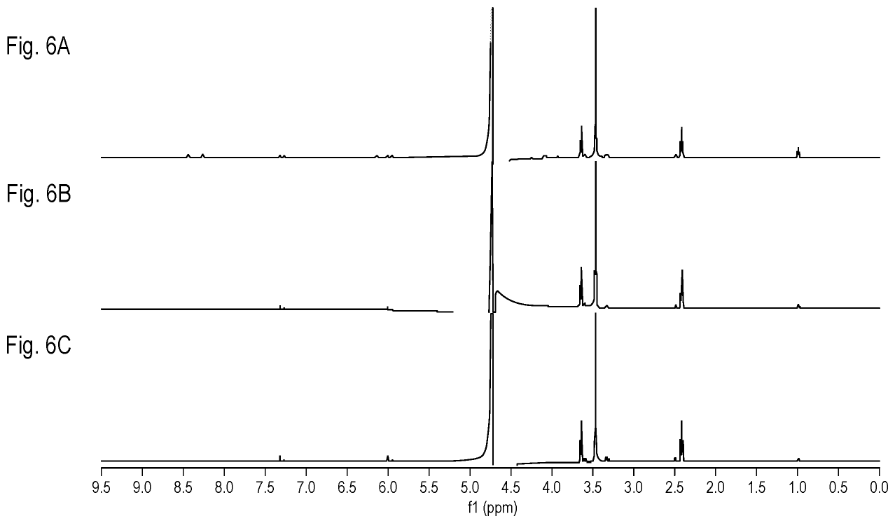

The two step synthesis of 3-HP from ACA is presented here as an original route to the target chemical. This in vitro pathway developed in this study utilizes the novel Cg10062 (E114N) hydratase-only mutant with two other enzymes, YdfG and PTDH (FIG. 1). YdfG is a NADP⁺-dependent 3-hydroxy acid dehydrogenase from E. coli and has previously been used for the in vivo production of 3-HP via the β-alanine pathway. When provided with NADPH as a cofactor, complete conversion of malonate semialdehyde to 3-HP was achieved. For our system to be applied practically, the cost of cofactor is an important consideration. NADPH is an expensive cofactor and in order for the pathway to be an efficient, cost-effective pathway, the use of sub-stoichiometric amounts of co-factor was carried out. An engineered phosphite dehydrogenase PTDH from Pseudomonas stutzeri (SEQ ID NO: 73) with the ability to reduce its non-native cofactor NADP⁺ was used to recycle cofactor for complete conversion of 100 mM ACA to 3-HP (FIG. 3). The data indicates that NADP(H) has an inhibitory effect on Cg10062 (E114N) and the hydration of ACA to MSA proceeds significantly faster at lower concentrations of NADP⁺. We were able to demonstrate successful and complete production of 100 mM 3-HP at concentrations as low as 0.001 eq NADP⁺. The formation of 3-HP in these assays were also confirmed using ¹H NMR (FIG. 4A-C). Furthermore, the assays were scaled to 500 mM ACA to demonstrate 3-HP synthesis using this pathway and was confirmed by HPLC and ¹H NMR analysis (FIGS. 5 and 6A-C). FIG. 5: Conversion of 500 mM ACA to 3-HP with cofactor recycling over a period of 61 h. FIG. 6A-C: ¹H NMR of 3-HP synthesis from 500 mM ACA with a) 0.1, b) 0.01 and c) 0.001 eq NADP(H). The conversion of 500 mM ACA to 3-HP was also demonstrated using the assay components as shown in Table 12.

Conversion of ACA to 3-HP was carried out on a 1 mL scale. All stocks solutions were prepared in 100 mM sodium phosphate pH 8.0. Ethylene glycol (20% w/v) was added to all enzyme stock solutions. For reactions containing 100 mM ACA, 4 reactions with varying amounts of NADP⁺ were carried out. The assay components for the four reactions are shown in Table 11. Each reaction was carried out in duplicate at 25° C. with constant slow mixing on a rocking platform. Reactions were initiated by the addition of ACA. Samples from each reaction were quenched at indicated timepoints for analysis by HPLC and ¹H NMR, as described below.

HPLC Analysis of 3-HP Synthesis: The conversion of ACA to 3-HP was confirmed by HPLC analysis using an Aminex HPX-87H column with 0.01 N sulfuric acid (mobile phase) and a flow rate of 0.6 mL min⁻¹ at 25° C. All samples (100 μL) were quenched and by addition of 5 μL of 18 M sulfuric acid. The final volume was adjusted to 500 μL using 0.01 N sulfuric acid. The samples were prepared for HPLC using Whatman Mini-UniPrep® G2 syringeless filters with a glass microfiber membrane.

¹H NMR Analysis of 3-HP Synthesis: ¹H NMR spectra were obtained at the beginning (t=0 h) and end of each assay. Reactions (100 μL) were combined with DMSO-d₆ (30 μL) and volume adjusted to 700 μL with 100 mM sodium phosphate buffer.

TABLE 11
Conversion of 100 mM ACA to 3-HP
with varying equivalents of NADP⁺.

| | ACA | Na₂HPO₃ | NADP⁺ | Cg10062(E114N) | YdfG | PTDH |
|---|---|---|---|---|---|---|
| Sample | (mM) | (mM) | (mM) | (U) | (U) | (U) |
| 1 eq. | 100 | 150 | 100 | 0.4 | 1.5 | 13 |
| 0.1 | 100 | 150 | 10 | 0.4 | 1.5 | 13 |
| eq. | | | | | | |
| 0.01 | 100 | 150 | 1 | 0.4 | 1.5 | 13 |
| eq. | | | | | | |
| 0.001 | 100 | 150 | 0.1 | 0.4 | 1.5 | 13 |
| eq. | | | | | | |

TABLE 12
Conversion of 500 mM ACA to 3-HP
with varying equivalents of NADP⁺.

| | ACA | Na₂HPO₃ | NADP⁺ | Cg10062(E114N) | YdfG | PTDH |
|---|---|---|---|---|---|---|
| Sample | (mM) | (mM) | (mM) | (U) | (U) | (U) |
| 0.1 eq. | 500 | 550 | 50 | 2 | 15 | 15 |
| 0.01 | 500 | 550 | 5 | 2 | 15 | 15 |
| eq. | | | | | | |
| 0.001 | 500 | 550 | 0.5 | 2 | 15 | 15 |
| eq. | | | | | | |

### Example 12: Synthesis of 3-HP In Vivo

Synthesis of 3-HP in vivo in Rich Media: A single colony of BL21/pAS(3-HP) was inoculated into 5 mL LB media and incubated at 37° C. for 12 h. The overnight culture was used to inoculate two 25 mL LB media to an initial OD₆₀₀ of 0.05. At an OD₆₀₀ of 0.5, only one of the cultures was induced by the addition of IPTG (100 mM) to a final concentration of 1 mM IPTG. A final concentration of 100 mM ACA pH 7.2 was added to both cultures when the OD₆₀₀ reached 0.5. The cultures were grown at 30° C. for 8 h. Aliquots (100 μL) of each culture was centrifuged at the time of IPTG induction (t=0 h) and 9 h after induction to remove cells. The samples were then analyzed with ¹H NMR using solvent suppression to remove HOD peak as described previously.

Synthesis of 3-HP in vivo in Minimal Media: A single colony of BL21/pAS(3-HP) was inoculated into 5 mL M9 media containing glucose and ampicillin and incubated at 37° C. for 12 h. The overnight culture was used to inoculate two 25 mL M9 cultures containing glucose and ampicillin to an initial OD₆₀₀ of 0.05. At an OD₆₀₀ of 0.5, only one of the cultures was induced by the addition of IPTG (100 mM) to a final concentration of 1 mM IPTG. Both cultures were returned to the shaker at 37° C. for 12 h. The cells were harvested and resuspended in sterile water to remove residual glucose. This step was repeated twice, and the cells were resuspended in fresh M9 media containing a final concentration of 100 mM ACA. The same cells that were previously induced with IPTG were re-induced with a final concentration of 1 mM IPTG and both cultures were grown for 72 h at 37° C.

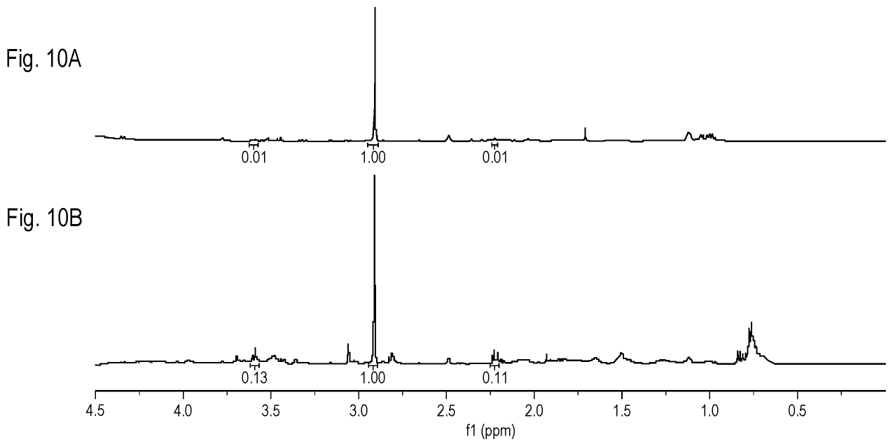

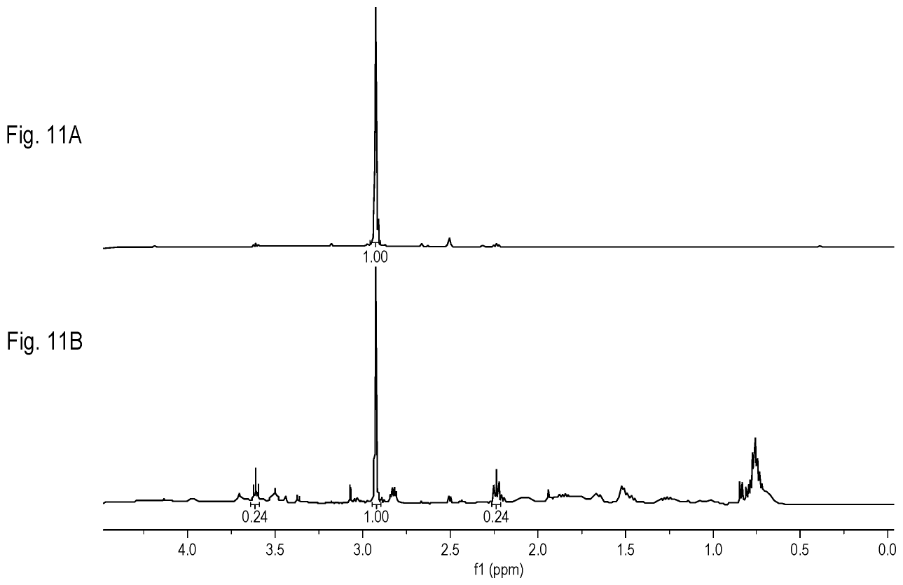

Synthesis of 3-HP in vivo with Cofactor Recycling: In preliminary studies, we were able to confirm the conversion of ACA to 3-HP using cells expressing Cg10062 (E114N) and YdfG. Trace amounts of 3-HP were observed in cultures grown in rich LB media and minimal M9 media (FIG. 10A-B and 11A-B). The amount of 3-HP formed in the uninduced minimal cultures were negligible relative to the 3-HP concentrations observed in the cultures induced with IPTG. 3-HP formation was also observed in cells grown in rich media. However, cultures that were not induced with IPTG also indicated the presence of 3-HP. This is likely a result of leaky expression of Cg10062 (E114N) and YdfG enzymes, as is commonly observed in rich media. See FIG. 10A-B. 3-HP formed from ACA in vivo in uninduced (A) and IPTG-induced (B) LB cultures. See FIG. 11A-B. 3-HP formed from ACA in vivo in IPTG-induced (B) M9 cultures. No 3-HP observed in uninduced (A) cultures.

### SH and MmsB Examples

### Media and Solutions

In addition to the media and solutions described in paragraphs [0171] and [0172], the following bacterial strains, genes and plasmids were used for the following experiments. The gene encoding MmsB was amplified from P. putida KT2440 genomic DNA and was cloned into the pET-21a(+) vector in the same way as YdfG as described in Example 1. C. necator Hf210/pGE771 was kindly provided by Professor Oliver Lenz of The Technical University of Berlin.

TABLE 13
Strains and plasmids used in the experiments below.

| | | Reference |
|---|---|---|
| Strain | Genotype/Description | [Source] |
| DH5a | lacZΔM15 hsdR recA | Invitrogen |
| BL21 | fhuA2 [lon] ompT gal | NEB |
| | [dcm] ΔhsdS | |
| BL21(DE3) | fhuA2 [lon] ompT gal | NEB |
| | (λ DE3) [dcm] Δ hsdS | |

| | | Reference |
|---|---|---|
| Plasmid | Description | [Source] |
| pET-21a(+) | Ap^{R}, P_{T7}, C-terminal His₆ | Novagen |
| pBbA1a- | Ap^{R}, P_{trc} | Addgene |
| RFP | | |
| pCg10062 | cg10062 in pET-21a(+) | this study |
| pYdfG | ydfg in pET-21a(+) | this study |
| pET-15b | pET-15b with Opt 12x ptdh and | Addgene |
| 12x | N-terminal His₆ | |
| pAS(3HP) | cg10062 and ydfg in pBbA1a-RFP | this study |
| pMmsB | mmsb in pET-21a(+) | this study |

### Example 13: SH Protein Expression, Purification and Quantification

SH was expressed and purified as described previously by Lenz, et al. Meth Enzymol; (2018); 613, 117-151, doi.org/10.1016/bs.mie.2018.10.008. Protein concentrations of cell lysates and purified enzyme were quantified as described in Example 3.

TABLE 14
Parameters used for enzyme quantification.

| Enzyme | MW (Da) | ε₂₈₀ (M⁻¹ cm⁻¹) |
|---|---|---|
| Cg10062 | 19013 | 30440 |
| Cg10062(E114Q) | 19012 | 30440 |
| Cg10062(E114D) | 18999 | 30440 |
| Cg10062(R70A) | 17142 | 29160 |
| Cg10062(R73A) | 17142 | 29160 |
| Cg10062(R70K) | 18985 | 30440 |
| Cg10062(R73K) | 18985 | 30440 |
| Cg10062(E114A) | 17169 | 29160 |
| Cg10062(H28A) | 17161 | 29160 |
| Cg10062(Y103A) | 18921 | 29160 |
| Cg10062(Y103F) | 18997 | 29160 |
| Cg10062(E114N) | 18998 | 30440 |
| YdfG | 28313 | 36130 |
| MSAD | 16464 | 8250 |
| 12x Opt PTDH | 36568 | 26600 |
| MmsB | 31386 | 17780 |

### Example 14: Kinetic Characterization of MmsB

MmsB was characterized using the coupled enzyme assay show in FIG. 28. All assays were carried out in triplicate at 25° C. in 100 mM potassium phosphate pH 8.0, at a final volume of 1 mL. All stock solutions used for the assays were prepared in 100 mM potassium phosphate pH 8.0. The specific activity of MmsB was measured by generating MSA in situ from the Cg10062 (E114N)-catalyzed hydration of ACA. The assay contained Cg10062 (E114N) (0.8 U), MmsB (0.001 mg mL−1, 10 μL of a 0.1 mg mL−1 stock) and NADH (0.1 mg/mL, 20 μL of a 5 mg mL−1 stock). ACA and Cg10062 (E114N) were mixed in buffer and left to sit 15 min before MmsB and NADH were added and oxidation of NADH was then followed at 340 nm. The initial rates of MmsB relative to varied ACA concentrations (50-10,000 μM) were plotted to fit the Michaelis-Menten model and analyzed using Origin 9.0 (FIG. 29). All other components and methods used for steady-state kinetics were identical to those described in Example 5.

### Example 15: Activity Characterization of SH

SH activity was monitored following the reduction of NAD⁺ at 365 nm (FIG. 30). The increase in absorbance at 365 nm, corresponding to the reduction of NAD+, was monitored at 0.1 s intervals and 25° C. To a cuvette, 50 mM Tris-HCl pH 8 and NAD+(40 μL, 0.004 nmol) were added. A septum was placed on the cuvette and it was tightly sealed using parafilm. H2 was bubbled through the solution for 2 minutes. A H2 filled balloon was attached to the cuvette and incubated in the UV-Vis for 2 minutes to ensure saturation. Soluble hydrogenase (SH) (10 μL, ˜0.2 U) was added through the septum via syringe to initiate the assay (2 mL final reaction volume).

### Example 16: pH Dependence of MmsB

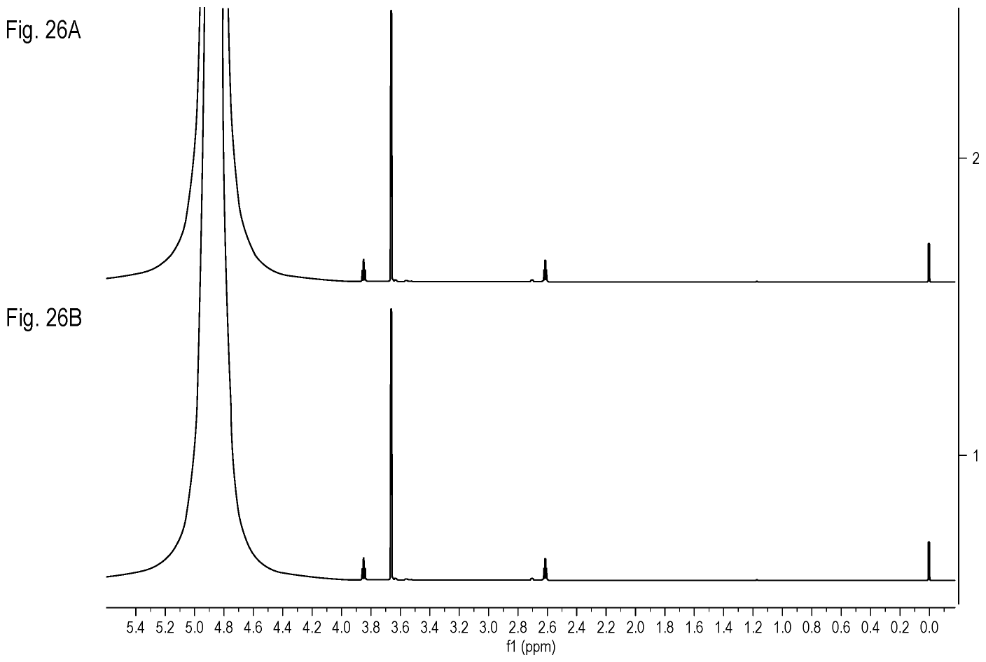

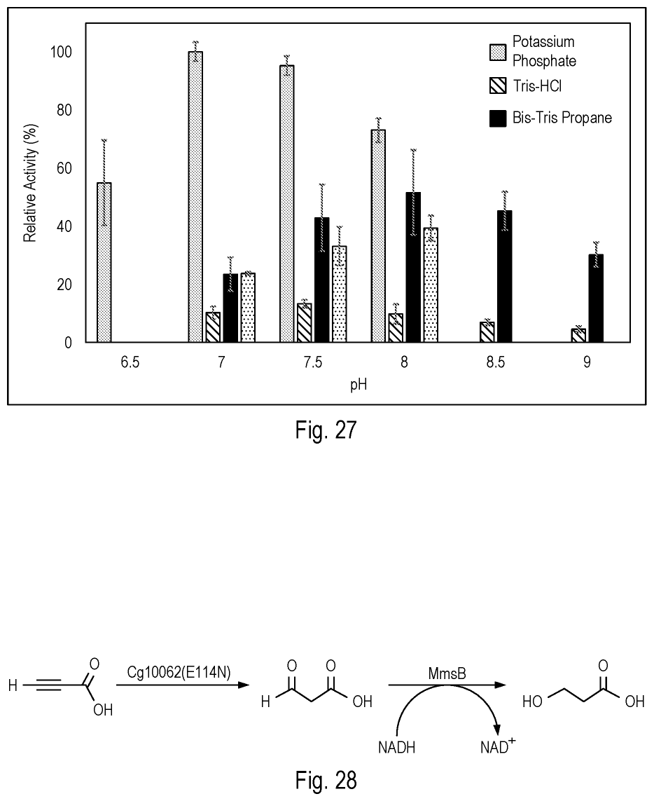

The pH dependence of MmsB was measured using four different buffer systems: 100 mM potassium phosphate, 50 mM Tris-HCl, 50 mM bis-tris propane and 50 mM HEPES buffers for pH 6.5-8.0, 7.0-9.0, 7.0-9.0 and 7.0-8.0, respectively. The pH dependence was studied using the respective enzyme assay used for kinetic characterization as described previously. All pH studies were carried out in triplicate (1 mL) on a Shimadzu UV2600 spectrophotometer at 25° C. and checked to ensure that the final pH of each assay remained unchanged with the addition of assay components. All stock solutions were prepared in water and the assays were carried out in the respective buffers for each pH. For each assay, buffer, ACA (5 mM, 50 μL of a 100 mM stock), and Cg10062 (E114N) (0.8 U) were combined and prepared in 1 mL microfuge tubes and incubated at 25° C. for at least 15 mins before MmsB (0.001 mg mL−1, 10 μL of a 0.1 mg mL−1 stock) and NADH (0.1 mg/mL, 20 μL of a 5 mg mL−1 stock) were added to initiate the reaction. Although MmsB shows highest activity at pH 7 in potassium phosphate, a system maintained at a pH of 8 was chosen due to the pH dependance of Cg10062 (E114N) and SH (FIG. 27).

### Example 17: Conversion of ACA to 3-HP with Cofactor Regeneration using MmsB and SH

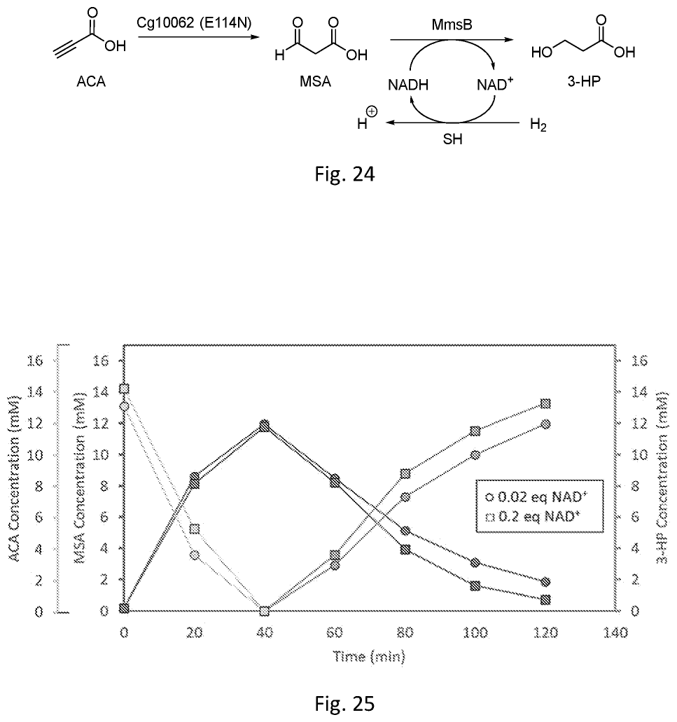

Another in vitro pathway has been developed to produce 3-HP from ACA. Cg10062 (E114N) is also used to hydrate ACA to MSA, but instead of YdfG, MmsB from P. putida KT2440 is used to reduce MSA to 3-HP, allowing for the use of NAD+ as cofactor (FIG. 24). Hydrogen gas is utilized to drive the recycling of NAD(H) using O2-tolerant and soluble NAD+-reducing hydrogenase from C. necator, allowing the possibility of using H2 formed during the dehydrodimerization of methane. Using 12.5 mM ACA, we were able to achieve complete conversion to 3-HP at an NAD+concentration as low as 0.02 eq. (FIG. 25). The conversion of ACA to 3-HP was carried out on a 4 mL scale. All stock solutions were prepared in 100 mM potassium phosphate pH 8.0. Two reactions with varying amounts of NAD⁺ were carried out. The assay components for the reactions are shown in Table 15. Each reaction was carried out in a sealed pear-shaped flask attached to a manifold. 100 mM potassium phosphate buffer pH 8, Cg10062 (E114N), and NAD⁺ were mixed and the reactions were initiated by the addition of ACA. At 40 minutes, MmsB was added and bubbling of hydrogen began. Samples from each reaction were quenched at indicated timepoints for analysis by ¹H NMR as described below.

TABLE 15
Conversion of 12.5 mM ACA to 3-HP
with varying equivalents of NAD⁺.

| | ACA | NAD⁺ | Cg10062(E114N) | MmsB | SH |
|---|---|---|---|---|---|
| Sample | (mM) | (mM) | (U) | (U) | (U) |
| 0.2 eq. | 12.5 | 2.5 | 3.1 | 0.7 | 1.5 |
| 0.02 eq | 12.5 | 0.25 | 3.1 | 0.7 | 1.5 |

### Example 18: ¹H NMR analysis of 3-HP synthesis using Cg10062 (E114N), MmsB, and SH

Samples of 490 μL were quenched with 10 μL sulfuric acid and added to 100 μL 10 mM 3-(Trimethylsilyl)propionic-2,2,3,3-d4 acid (TSP) in D2O. 1H NMR spectra were obtained using 64 scans and 10 s relaxation delays. Concentrations were calculated using TSP as internal standard. Polynomial baseline correction was used on each spectra. ¹H NMR of 3-HP synthesis from 12.5 mM ACA with NAD(H) is shown in FIG. 26A-B.

| SEQ ID | | |
|---|---|---|
| NO | Description | Sequence |
| 1 | Cg10062 (NCBI: | ATGCCGACCTACACCTGCTGGAGCCAAC |
| | MZ369159) from | GCATTCGTATTAGCCGTGAAGCGAAGCA |
| | Corynebacterium | ACGCATCGCGGAAGCGATTACCGACGCG |
| | glutamicum codon- | CACCATGAACTGGCGCACGCGCCGAAGT |
| | optimized for | ACCTGGTGCAGGTTATTTTCAACGAAGT |
| | Escherichia coli | GGAGCCGGACAGCTATTTTATCGCGGCG |
| | | CAGAGCGCGAGCGAGAACCACATTTGG |
| | | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AA |
| 2 | Cg10062(E114Q) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli | ACCTGGTGCAGGTTATTTTCAACGAAGT |
| | | GGAGCCGGACAGCTATTTTATCGCGGCG |
| | | CAGAGCGCGAGCGAGAACCACATTTGG |
| | | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCCAGTATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AA |
| 3 | Cg10062(E114D) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli | ACCTGGTGCAGGTTATTTTCAACGAAGT |
| | | GGAGCCGGACAGCTATTTTATCGCGGCG |
| | | CAGAGCGCGAGCGAGAACCACATTTGG |
| | | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGATTATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AA |
| 4 | Cg10062(E114N) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli | ACCTGGTGCAGGTTATTTTCAACGAAGT |
| | | GGAGCCGGACAGCTATTTTATCGCGGCG |
| | | CAGAGCGCGAGCGAGAACCACATTTGG |
| | | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCAACTATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AA |
| 5 | Cg10062(H28A) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | GCGCATGAACTGGCGCACGCGCCGAAGT |
| | Escherichia coli | ACCTGGTGCAGGTTATTTTCAACGAAGT |
| | | GGAGCCGGACAGCTATTTTATCGCGGCG |
| | | CAGAGCGCGAGCGAGAACCACATTTGG |
| | | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AA |
| 6 | Cg10062(R70A) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli | ACCTGGTGCAGGTTATTTTCAACGAAGT |
| | | GGAGCCGGACAGCTATTTTATCGCGGCG |
| | | CAGAGCGCGAGCGAGAACCACATTTGG |
| | | GTTCAAGCGACCATCGCGAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AA |
| 7 | Cg10062(R70K) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli | ACCTGGTGCAGGTTATTTTCAACGAAGT |
| | | GGAGCCGGACAGCTATTTTATCGCGGCG |
| | | CAGAGCGCGAGCGAGAACCACATTTGG |
| | | GTTCAAGCGACCATCAAAAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AA |
| 8 | Cg10062(R73A) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli | ACCTGGTGCAGGTTATTTTCAACGAAGT |
| | | GGAGCCGGACAGCTATTTTATCGCGGCG |
| | | CAGAGCGCGAGCGAGAACCACATTTGG |
| | | GTTCAAGCGACCATCCGTAGCGGCGCGA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AA |
| 9 | Cg10062(R73K) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli | ACCTGGTGCAGGTTATTTTCAACGAAGT |
| | | GGAGCCGGACAGCTATTTTATCGCGGCG |
| | | CAGAGCGCGAGCGAGAACCACATTTGG |
| | | GTTCAAGCGACCATCCGTAGCGGCAAAA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AA |
| 10 | Cg10062(Y103A) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli | ACCTGGTGCAGGTTATTTTCAACGAAGT |
| | | GGAGCCGGACAGCTATTTTATCGCGGCG |
| | | CAGAGCGCGAGCGAGAACCACATTTGG |
| | | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTGCGATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AA |
| 11 | Cg10062(Y103F) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli | ACCTGGTGCAGGTTATTTTCAACGAAGT |
| | | GGAGCCGGACAGCTATTTTATCGCGGCG |
| | | CAGAGCGCGAGCGAGAACCACATTTGG |
| | | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTTTATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AA |
| 12 | Cg10062(E114A) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli | ACCTGGTGCAGGTTATTTTCAACGAAGT |
| | | GGAGCCGGACAGCTATTTTATCGCGGCG |
| | | CAGAGCGCGAGCGAGAACCACATTTGG |
| | | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGCGTATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AA |
| 13 | Cg10062(E114D- | ATGCCGACCTACACCTGCTGGAGCCAAC |
| | Y103F) from | GCATTCGTATTAGCCGTGAAGCGAAGCA |
| | Corynebacterium | ACGCATCGCGGAAGCGATTACCGACGCG |
| | glutamicum codon- | CACCATGAACTGGCGCACGCGCCGAAGT |
| | optimized for | ACCTGGTGCAGGTTATTTTCAACGAAGT |
| | Escherichia coli | GGAGCCGGACAGCTATTTTATCGCGGCG |
| | | CAGAGCGCGAGCGAGAACCACATTTGG |
| | | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTTTATTACCGAAATCCCGGGTAGCA |
| | | ACATGACCGATTATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AA |
| 14 | Cis-CaaD from | ATGCCGGTTTACATGGTTTACGTTAGCC |
| | Coryneform sp. codon- | AGGACCGTCTGACCCCGAGCGCGAAGC |
| | optimized for | ACGCGGTTGCGAAGGCGATTACCGATGC |
| | expression in | GCACCGTGGTCTGACCGGCACCCAGCAC |
| | Escherichia coli | TTCCTGGCGCAAGTGAACTTTCAGGAGC |
| | | AACCGGCGGGTAACGTGTTCCTGGGTGG |
| | | CGTTCAGCAAGGTGGCGACACCATCTTT |
| | | GTTCATGGTCTGCACCGTGAGGGCCGTA |
| | | GCGCGGATCTGAAGGGCCAGCTGGCGC |
| | | AACGTATTGTTGACGATGTGAGCGTTGC |
| | | GGCGGAAATCGACCGTAAACACATTTGG |
| | | GTGTACTTCGGCGAGATGCCGGCGCAGC |
| | | AAATGGTTGAATATGGCCGTTTCCTGCC |
| | | GCAGCCGGGTCATGAGGGTGAATGGTTT |
| | | GACAACCTGAGCAGCGATGAACGTGCGT |
| | | TTATGGAGACCAATGTTGATGTGAGCCG |
| | | TACC |
| 15 | Engineered phosphite | ATGCTGCCGAAACTCGTTATAACTCACC |
| | dehydrogenase (PTDH) | GAGTACACGAAGAGATCCTGCAACTGCT |
| | from Pseudomonas | GGCGCCACATTGCGAGCTGATAACCAAC |
| | stutzeri | CAGACCGACAGCACGCTGACGCGCGAG |
| | | GAAATTCTGCGCCGCTGTCGCGATGCTC |
| | | AGGCGATGATGGCGTTCATGCCCGATCG |
| | | GGTCGATGCAGACTTTCTTCAAGCCTGC |
| | | CCTGAGCTGCGTGTAATCGGCTGCGCGC |
| | | TCAAGGGCTTCGACAATTTCGATGTGGA |
| | | CGCCTGTACTGCCCGCGGGGTCTGGCTG |
| | | ACCTTCGTGCCTGATCTGTTGACGGTCCC |
| | | GACTGCCGAGCTGGCGATCGGACTGGCG |
| | | GTGGGGCTGGGGCGGCATCTGCGGGCA |
| | | GCAGATGCGTTCGTCCGCTCTGGCAAGT |
| | | TCCGGGGCTGGCAACCACGGTTCTACGG |
| | | CACGGGGCTGGATAACGCTACGGTCGGC |
| | | TTCCTTGGCATGGGCGCCATCGGACTGG |
| | | CCATGGCTGATCGCTTGCAGGGATGGGG |
| | | CGCGACCCTGCAGTACCACGCGGCGAAG |
| | | GCTCTGGATACACAAACCGAGCAACGGC |
| | | TCGGCCTGCGCCAGGTGGCGTGCAGCGA |
| | | ACTCTTCGCCAGCTCGGACTTCATCCTGC |
| | | TGGCGCTTCCCTTGAATGCCGATACCCT |
| | | GCATCTGGTCAACGCCGAGCTGCTTGCC |
| | | CTCGTACGGCCGGGCGCTCTGCTTGTAA |
| | | ACCCCTGTCGTGGCTCGGTAGTGGATGA |
| | | AGCCGCCGTGCTCGCGGCGCTTGAGCGA |
| | | GGCCAGCTCGGCGGGTATGCGGCGGATG |
| | | TATTCGAAATGGAAGACTGGGCTCGCGC |
| | | GGACCGGCCGCAGCAGATCGATCCTGCG |
| | | CTGCTCGCGCATCCGAATACGCTGTTCA |
| | | CTCCGCACATAGGGTCGGCAGTGCGCGC |
| | | GGTGCGCCTGGAGATTGAACGTTGTGCA |
| | | GCGCAGAACATCCTCCAGGCATTGGCAG |
| | | GTGAGCGCCCAATCAACGCTGTGAACCG |
| | | TCTGCCCAAGGCCAATCCTGCCGCAGAC |
| 16 | Malonate semialdehyde | ATGCCCTTAATCCGTATAGATCTTACCA |
| | decarboxylase (MSAD) | GTGATCGTTCGAGAGAGCAACGGCGGG |
| | from Coryneform sp. | CGATTGCTGATGCAGTCCATGACGCTTT |
| | FG41 (NCBI: | AGTAGAAGTTTTAGCGATTCCGGCTCGT |
| | MZ369160) | GATCGCTTCCAGATACTGACTGCGCACG |
| | | ATCCCTCTGATATTATAGCCGAAGATGC |
| | | TGGACTTGGCTTTCAGCGGTCCCCCAGT |
| | | GTAGTCATCATACACGTCTTTACACAGG |
| | | CAGGTAGAACTATTGAAACGAAACAGA |
| | | GAGTATTTGCAGCGATAACAGAAAGTCT |
| | | GGCTCCAATCGGTGTTGCAGGATCTGAT |
| | | GTTTTTATCGCAATCACCGAAAATGCAC |
| | | CCCATGACTGGAGCTTTGGGTTTGGCAG |
| | | TGCACAATATGTCACGGGTGAACTTGCG |
| | | ATTCCAGCCACTGGTGCGGCT |
| 17 | YdfG from Escherichia | ATGATCGTTTTAGTAACTGGAGCAACGG |
| | coli | CAGGTTTTGGTGAATGCATTACTCGTCG |
| | | TTTTATTCAACAAGGGCATAAAGTTATC |
| | | GCCACTGGCCGTCGCCAGGAACGGTTGC |
| | | AGGAGTTAAAAGACGAACTGGGAGATA |
| | | ATCTGTATATCGCCCAACTGGACGTTCG |
| | | CAACCGCGCCGCTATTGAAGAGATGCTG |
| | | GCATCGCTTCCTGCCGAGTGGTGCAATA |
| | | TTGATATCCTGGTAAATAATGCCGGCCT |
| | | GGCGTTGGGCATGGAGCCTGCGCATAAA |
| | | GCCAGCGTTGAAGACTGGGAAACGATG |
| | | ATTGATACCAACAACAAAGGCCTGGTAT |
| | | ATATGACGCGCGCCGTCTTACCGGGTAT |
| | | GGTTGAACGTAATCATGGTCATATTATT |
| | | AACATTGGCTCAACGGCAGGTAGCTGGC |
| | | CGTATGCCGGTGGTAACGTTTACGGTGC |
| | | GACGAAAGCGTTTGTTCGTCAGTTTAGC |
| | | CTGAATCTGCGTACGGATCTGCATGGTA |
| | | CGGCGGTGCGCGTCACCGACATCGAACC |
| | | GGGTCTGGTGGGTGGTACCGAGTTTTCC |
| | | AATGTCCGCTTTAAAGGCGATGACGGTA |
| | | AAGCAGAAAAAACCTATCAAAATACCG |
| | | TTGCATTGACGCCAGAAGATGTCAGCGA |
| | | AGCCGTCTGGTGGGTGTCAACGCTGCCT |
| | | GCTCACGTCAATATCAATACCCTGGAAA |
| | | TGATGCCGGTTACCCAAAGCTATGCCGG |
| | | ACTGAATGTCCACCGTCAG |
| 18 | MmsB from | ATGCGTATCGCATTCATCGGCCTGGGCA |
| | Pseudomonas putida | ACATGGGCGCGCCCATGGCCCGCAACCT |
| | KT2440 | GATCAAGGCCGGGCATCAGCTGAACCTG |
| | | TTCGACCTGAACAAGGCCGTGCTGGCCG |
| | | AGCTGGCAGAACTGGGCGGGCAGATCA |
| | | GCCCCTCGCCCAAGGACGCGGCGGCCAA |
| | | CAGCGAGCTGGTGATCACCATGCTGCCG |
| | | GCCGCAGCCCATGTGCGTAGCGTGTACT |
| | | TGAACGAGGACGGCGTACTGGCCGGTAT |
| | | TCGTCCTGGCACGCCGACCGTTGACTGC |
| | | AGCACCATCGACCCGCAGACCGCACGTG |
| | | ACGTGTCCAAGGCCGCAGCGGCAAAGG |
| | | GCGTGGACATGGGGGATGCGCCGGTTTC |
| | | CGGTGGTACTGGCGGCGCGGCGGCCGGC |
| | | ACCCTGACGTTCATGGTCGGCGCCAGTA |
| | | CCGAGTTGTTCGCCAGCCTCAAGCCGGT |
| | | ACTGGAGCAGATGGGCCGCAACATCGTG |
| | | CACTGCGGGGAAGTCGGTACCGGCCAG |
| | | ATCGCCAAGATCTGCAACAACCTGCTGC |
| | | TCGGCATTTCGATGATCGGCGTGTCCGA |
| | | GGCCATGGCCCTGGGTAACGCGCTGGGT |
| | | ATCGATACCAAGGTGCTGGCCGGCATCA |
| | | TCAACAGTTCGACCGGGCGTTGCTGGAG |
| | | CTCGGACACCTACAACCCGTGGCCGGGC |
| | | ATCATCGAAACCGCACCTGCATCGCGTG |
| | | GCTACACCGGTGGCTTTGGCGCCGAACT |
| | | CATGCTCAAGGACCTGGGGTTGGCCACC |
| | | GAAGCGGCACGCCAGGCACACCAACCG |
| | | GTGATTCTCGGTGCCGTGGCCCAGCAGC |
| | | TGTACCAGGCCATGAGCCTGCGAGGCGA |
| | | GGGTGGCAAGGACTTCTCGGCCATCGTC |
| | | GAGGGTTATCGCAAGAAGGAT |
| 19 | PTDH N-terminal His₆- | ATGGGCAGCAGCCATCATCATCATCATC |
| | tag from pET-15b | ACAGCAGCGGCCTGGTGCCGCGCGGCA |
| | vector | GCCAT |
| 20 | TEV protease | GAGAACCTGTATTTTCAAGGCCTCGAGC |
| | recognition site and C- | ACCACCACCACCACCAC |
| | terminal His₆-tag | |
| 21 | Cg10062 from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVYITEIPGSNMTEYGRLLM |
| | Escherichia coli | EPGEEEKWFNSLPEGLRERLTELEGSSE |
| 22 | Cg10062(E114Q) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVYITEIPGSNMTQYGRLLM |
| | Escherichia coli | EPGEEEKWFNSLPEGLRERLTELEGSSE |
| 23 | Cg10062(E114D) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVYITEIPGSNMTDYGRLLM |
| | Escherichia coli | EPGEEEKWFNSLPEGLRERLTELEGSSE |
| 24 | Cg10062(E114N) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVYITEIPGSNMTNYGRLLM |
| | Escherichia coli | EPGEEEKWFNSLPEGLRERLTELEGSSE |
| 25 | Cg10062(H28A) from | PTYTCWSQRIRISREAKQRIAEAITDAAHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVYITEIPGSNMTEYGRLLM |
| | Escherichia coli | EPGEEEKWFNSLPEGLRERLTELEGSSE |
| 26 | Cg10062(R70A) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIASGRTEKQKEELLLRLTQEIA |
| | optimized for | LILGIPNEEVWVYITEIPGSNMTEYGRLLM |
| | Escherichia coli | EPGEEEKWFNSLPEGLRERLTELEGSSE |
| 27 | Cg10062(R70K) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIKSGRTEKQKEELLLRLTQEIA |
| | optimized for | LILGIPNEEVWVYITEIPGSNMTEYGRLLM |
| | Escherichia coli | EPGEEEKWFNSLPEGLRERLTELEGSSE |
| 28 | Cg10062(R73A) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGATEKQKEELLLRLTQEIA |
| | optimized for | LILGIPNEEVWVYITEIPGSNMTEYGRLLM |
| | Escherichia coli | EPGEEEKWFNSLPEGLRERLTELEGSSE |
| 29 | Cg10062(R73K) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGKTEKQKEELLLRLTQEIA |
| | optimized for | LILGIPNEEVWVYITEIPGSNMTEYGRLLM |
| | Escherichia coli | EPGEEEKWFNSLPEGLRERLTELEGSSE |
| 30 | Cg10062(Y103A) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVAITEIPGSNMTEYGRLLM |
| | Escherichia coli | EPGEEEKWFNSLPEGLRERLTELEGSSE |
| 31 | Cg10062(Y103F) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVFITEIPGSNMTEYGRLLM |
| | Escherichia coli | EPGEEEKWFNSLPEGLRERLTELEGSSE |
| 32 | Cg10062(E114A) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVYITEIPGSNMTAYGRLLM |
| | Escherichia coli | EPGEEEKWFNSLPEGLRERLTELEGSSE |
| 33 | Cg10062(E114D- | PTYTCWSQRIRISREAKQRIAEAITDAHHE |
| | Y103F) from | LAHAPKYLVQVIFNEVEPDSYFIAAQSASE |
| | Corynebacterium | NHIWVQATIRSGRTEKQKEELLLRLTQEIA |
| | glutamicum codon- | LILGIPNEEVWVFITEIPGSNMTDYGRLLM |
| | optimized for | EPGEEEKWFNSLPEGLRERLTELEGSSE |
| | Escherichia coli | |
| 34 | Cis-CaaD from | PVYMVYVSQDRLTPSAKHAVAKAITDAH |
| | Coryneform sp. codon- | RGLTGTQHFLAQVNFQEQPAGNVFLGGV |
| | optimized for | QQGGDTIFVHGLHREGRSADLKGQLAQRI |
| | expression in | VDDVSVAAEIDRKHIWVYFGEMPAQQMV |
| | Escherichia coli | EYGRFLPQPGHEGEWFDNLSSDERAFMET |
| | | NVDVSRT |
| 35 | Engineered phosphite | MLPKLVITHRVHEEILQLLAPHCELITNQT |
| | dehydrogenase (PTDH) | DSTLTREEILRRCRDAQAMMAFMPDRVD |
| | from Pseudomonas | ADFLQACPELRVIGCALKGFDNFDVDACT |
| | stutzeri | ARGVWLTFVPDLLTVPTAELAIGLAVGLG |
| | | RHLRAADAFVRSGKFRGWQPRFYGTGLD |
| | | NATVGFLGMGAIGLAMADRLQGWGATL |
| | | QYHAAKALDTQTEQRLGLRQVACSELFAS |
| | | SDFILLALPLNADTLHLVNAELLALVRPGA |
| | | LLVNPCRGSVVDEAAVLAALERGQLGGY |
| | | AADVFEMEDWARADRPQQIDPALLAHPN |
| | | TLFTPHIGSAVRAVRLEIERCAAQNILQAL |
| | | AGERPINAVNRLPKANPAAD |
| 36 | Malonate semialdehyde | PLIRIDLTSDRSREQRRAIADAVHDALVEV |
| | decarboxylase (MSAD) | LAIPARDRFQILTAHDPSDIIAEDAGLGFQR |
| | from Coryneform sp. | SPSVVIIHVFTQAGRTIETKQRVFAAITESL |
| | FG41 | APIGVAGSDVFIAITENAPHDWSFGFGSAQ |
| | | YVTGELAIPATGAA |
| 37 | YdfG from Escherichia | MIVLVTGATAGFGECITRRFIQQGHKVIAT |
| | coli | GRRQERLQELKDELGDNLYIAQLDVRNRA |
| | | AIEEMLASLPAEWCNIDILVNNAGLALGM |
| | | EPAHKASVEDWETMIDTNNKGLVYMTRA |
| | | VLPGMVERNHGHIINIGSTAGSWPYAGGN |
| | | VYGATKAFVRQFSLNLRTDLHGTAVRVT |
| | | DIEPGLVGGTEFSNVRFKGDDGKAEKTYQ |
| | | NTVALTPEDVSEAVWWVSTLPAHVNINTL |
| | | EMMPVTQSYAGLNVHRQ |
| 38 | MmsB from | MRIAFIGLGNMGAPMARNLIKAGHQLNLF |
| | Pseudomonas putida | DLNKAVLAELAELGGQISPSPKDAAANSE |
| | KT2440 | LVITMLPAAAHVRSVYLNEDGVLAGIRPG |
| | | TPTVDCSTIDPQTARDVSKAAAAKGVDM |
| | | GDAPVSGGTGGAAAGTLTFMVGASTELF |
| | | ASLKPVLEQMGRNIVHCGEVGTGQIAKIC |
| | | NNLLLGISMIGVSEAMALGNALGIDTKVL |
| | | AGIINSSTGRCWSSDTYNPWPGIIETAPASR |
| | | GYTGGFGAELMLKDLGLATEAARQAHQP |
| | | VILGAVAQQLYQAMSLRGEGGKDFSAIVE |
| | | GYRKKD |
| 39 | PTDH N-terminal His₆- | MGSSHHHHHHSSGLVPRGSH |
| | tag from pET-15b | |
| | vector | |
| 40 | TEV protease | ENLYFQGLEHHHHHH |
| | recognition site and C- | |
| | terminal His₆-tag | |
| 41 | Cg10062 (NCBI: | ATGCCGACCTACACCTGCTGGAGCCAAC |
| | MZ369159) from | GCATTCGTATTAGCCGTGAAGCGAAGCA |
| | Corynebacterium | ACGCATCGCGGAAGCGATTACCGACGCG |
| | glutamicum codon- | CACCATGAACTGGCGCACGCGCCGAAGT |
| | optimized for | ACCTGGTGCAGGTTATTTTCAACGAAGT |
| | Escherichia coli plus | GGAGCCGGACAGCTATTTTATCGCGGCG |
| | TEV protease | CAGAGCGCGAGCGAGAACCACATTTGG |
| | recognition site and C- | GTTCAAGCGACCATCCGTAGCGGCCGTA |
| | terminal His₆-tag | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AAGAGAACCTGTATTTTCAAGGCCTCGA |
| | | GCACCACCACCACCACCAC |
| 42 | Cg10062(E114Q) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli plus | ACCTGGTGCAGGTTATTTTCAACGAAGT |
| | TEV protease | GGAGCCGGACAGCTATTTTATCGCGGCG |
| | recognition site and C- | CAGAGCGCGAGCGAGAACCACATTTGG |
| | terminal His₆-tag | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCCAGTATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AAGAGAACCTGTATTTTCAAGGCCTCGA |
| | | GCACCACCACCACCACCAC |
| 43 | Cg10062(E114D) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli plus | ACCTGGTGCAGGTTATTTTCAACGAAGT |
| | TEV protease | GGAGCCGGACAGCTATTTTATCGCGGCG |
| | recognition site and C- | CAGAGCGCGAGCGAGAACCACATTTGG |
| | terminal His₆-tag | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGATTATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AAGAGAACCTGTATTTTCAAGGCCTCGA |
| | | GCACCACCACCACCACCAC |
| 44 | Cg10062(E114N) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli plus | ACCTGGTGCAGGTTATTTTCAACGAAGT |
| | TEV protease | GGAGCCGGACAGCTATTTTATCGCGGCG |
| | recognition site and C- | CAGAGCGCGAGCGAGAACCACATTTGG |
| | terminal His₆-tag | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCAACTATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AAGAGAACCTGTATTTTCAAGGCCTCGA |
| | | GCACCACCACCACCACCAC |
| 45 | Cg10062(H28A) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | GCGCATGAACTGGCGCACGCGCCGAAGT |
| | Escherichia coli plus | ACCTGGTGCAGGTTATTTTCAACGAAGT |
| | TEV protease | GGAGCCGGACAGCTATTTTATCGCGGCG |
| | recognition site and C- | CAGAGCGCGAGCGAGAACCACATTTGG |
| | terminal His₆-tag | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AAGAGAACCTGTATTTTCAAGGCCTCGA |
| | | GCACCACCACCACCACCAC |
| 46 | Cg10062(R70A) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli plus | ACCTGGTGCAGGTTATTTTCAACGAAGT |
| | TEV protease | GGAGCCGGACAGCTATTTTATCGCGGCG |
| | recognition site and C- | CAGAGCGCGAGCGAGAACCACATTTGG |
| | terminal His₆-tag | GTTCAAGCGACCATCGCGAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AAGAGAACCTGTATTTTCAAGGCCTCGA |
| | | GCACCACCACCACCACCAC |
| 47 | Cg10062(R70K) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli plus | ACCTGGTGCAGGTTATTTTCAACGAAGT |
| | TEV protease | GGAGCCGGACAGCTATTTTATCGCGGCG |
| | recognition site and C- | CAGAGCGCGAGCGAGAACCACATTTGG |
| | terminal His₆-tag | GTTCAAGCGACCATCAAAAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AAGAGAACCTGTATTTTCAAGGCCTCGA |
| | | GCACCACCACCACCACCAC |
| 48 | Cg10062(R73A) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli plus | ACCTGGTGCAGGTTATTTTCAACGAAGT |
| | TEV protease | GGAGCCGGACAGCTATTTTATCGCGGCG |
| | recognition site and C- | CAGAGCGCGAGCGAGAACCACATTTGG |
| | terminal His₆-tag | GTTCAAGCGACCATCCGTAGCGGCGCGA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AAGAGAACCTGTATTTTCAAGGCCTCGA |
| | | GCACCACCACCACCACCAC |
| 49 | Cg10062(R73K) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli plus | ACCTGGTGCAGGTTATTTTCAACGAAGT |
| | TEV protease | GGAGCCGGACAGCTATTTTATCGCGGCG |
| | recognition site and C- | CAGAGCGCGAGCGAGAACCACATTTGG |
| | terminal His₆-tag | GTTCAAGCGACCATCCGTAGCGGCAAAA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AAGAGAACCTGTATTTTCAAGGCCTCGA |
| | | GCACCACCACCACCACCAC |
| 50 | Cg10062(Y103A) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli plus | ACCTGGTGCAGGTTATTTTCAACGAAGT |
| | TEV protease | GGAGCCGGACAGCTATTTTATCGCGGCG |
| | recognition site and C- | CAGAGCGCGAGCGAGAACCACATTTGG |
| | terminal His₆-tag | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTGCGATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AAGAGAACCTGTATTTTCAAGGCCTCGA |
| | | GCACCACCACCACCACCAC |
| 51 | Cg10062(Y103F) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli plus | ACCTGGTGCAGGTTATTTTCAACGAAGT |
| | TEV protease | GGAGCCGGACAGCTATTTTATCGCGGCG |
| | recognition site and C- | CAGAGCGCGAGCGAGAACCACATTTGG |
| | terminal His₆-tag | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTTTATTACCGAAATCCCGGGTAGCA |
| | | ACATGACCGAATATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AAGAGAACCTGTATTTTCAAGGCCTCGA |
| | | GCACCACCACCACCACCAC |
| 52 | Cg10062(E114A) from | ATGCCGACCTACACCTGCTGGAGCCAAC |
| | Corynebacterium | GCATTCGTATTAGCCGTGAAGCGAAGCA |
| | glutamicum codon- | ACGCATCGCGGAAGCGATTACCGACGCG |
| | optimized for | CACCATGAACTGGCGCACGCGCCGAAGT |
| | Escherichia coli plus | ACCTGGTGCAGGTTATTTTCAACGAAGT |
| | TEV protease | GGAGCCGGACAGCTATTTTATCGCGGCG |
| | recognition site and C- | CAGAGCGCGAGCGAGAACCACATTTGG |
| | terminal His₆-tag | GTTCAAGCGACCATCCGTAGCGGCCGTA |
| | | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTACATTACCGAAATCCCGGGTAGCA |
| | | ACATGACCGCGTATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AAGAGAACCTGTATTTTCAAGGCCTCGA |
| | | GCACCACCACCACCACCAC |
| 53 | Cg10062(E114D- | ATGCCGACCTACACCTGCTGGAGCCAAC |
| | Y103F) from | GCATTCGTATTAGCCGTGAAGCGAAGCA |
| | Corynebacterium | ACGCATCGCGGAAGCGATTACCGACGCG |
| | glutamicum codon- | CACCATGAACTGGCGCACGCGCCGAAGT |
| | optimized for | ACCTGGTGCAGGTTATTTTCAACGAAGT |
| | Escherichia coli plus | GGAGCCGGACAGCTATTTTATCGCGGCG |
| | TEV protease | CAGAGCGCGAGCGAGAACCACATTTGG |
| | recognition site and C- | GTTCAAGCGACCATCCGTAGCGGCCGTA |
| | terminal His₆-tag | CCGAAAAGCAGAAAGAGGAACTGCTGC |
| | | TGCGTCTGACCCAAGAGATCGCGCTGAT |
| | | TCTGGGTATCCCGAACGAGGAAGTGTGG |
| | | GTTTTTATTACCGAAATCCCGGGTAGCA |
| | | ACATGACCGATTATGGCCGTCTGCTGAT |
| | | GGAGCCGGGCGAGGAAGAGAAATGGTT |
| | | CAACAGCCTGCCGGAGGGCCTGCGTGAG |
| | | CGTCTGACCGAACTGGAGGGTAGCAGCG |
| | | AAGAGAACCTGTATTTTCAAGGCCTCGA |
| | | GCACCACCACCACCACCAC |
| 54 | Cis-CaaD from | ATGCCGGTTTACATGGTTTACGTTAGCC |
| | Coryneform sp. codon- | AGGACCGTCTGACCCCGAGCGCGAAGC |
| | optimized for | ACGCGGTTGCGAAGGCGATTACCGATGC |
| | Escherichia coli plus | GCACCGTGGTCTGACCGGCACCCAGCAC |
| | TEV protease | TTCCTGGCGCAAGTGAACTTTCAGGAGC |
| | recognition site and C- | AACCGGCGGGTAACGTGTTCCTGGGTGG |
| | terminal His₆-tag | CGTTCAGCAAGGTGGCGACACCATCTTT |
| | | GTTCATGGTCTGCACCGTGAGGGCCGTA |
| | | GCGCGGATCTGAAGGGCCAGCTGGCGC |
| | | AACGTATTGTTGACGATGTGAGCGTTGC |
| | | GGCGGAAATCGACCGTAAACACATTTGG |
| | | GTGTACTTCGGCGAGATGCCGGCGCAGC |
| | | AAATGGTTGAATATGGCCGTTTCCTGCC |
| | | GCAGCCGGGTCATGAGGGTGAATGGTTT |
| | | GACAACCTGAGCAGCGATGAACGTGCGT |
| | | TTATGGAGACCAATGTTGATGTGAGCCG |
| | | TACCGAGAACCTGTATTTTCAAGGCCTC |
| | | GAGCACCACCACCACCACCAC |
| 55 | Engineered phosphite | ATGGGCAGCAGCCATCATCATCATCATC |
| | dehydrogenase (PTDH) | ACAGCAGCGGCCTGGTGCCGCGCGGCA |
| | from Pseudomonas | GCCATATGCTGCCGAAACTCGTTATAAC |
| | stutzeri plus N-terminal | TCACCGAGTACACGAAGAGATCCTGCAA |
| | His₆-tag from pET-15b | CTGCTGGCGCCACATTGCGAGCTGATAA |
| | vector | CCAACCAGACCGACAGCACGCTGACGC |
| | | GCGAGGAAATTCTGCGCCGCTGTCGCGA |
| | | TGCTCAGGCGATGATGGCGTTCATGCCC |
| | | GATCGGGTCGATGCAGACTTTCTTCAAG |
| | | CCTGCCCTGAGCTGCGTGTAATCGGCTG |
| | | CGCGCTCAAGGGCTTCGACAATTTCGAT |
| | | GTGGACGCCTGTACTGCCCGCGGGGTCT |
| | | GGCTGACCTTCGTGCCTGATCTGTTGAC |
| | | GGTCCCGACTGCCGAGCTGGCGATCGGA |
| | | CTGGCGGTGGGGCTGGGGCGGCATCTGC |
| | | GGGCAGCAGATGCGTTCGTCCGCTCTGG |
| | | CAAGTTCCGGGGCTGGCAACCACGGTTC |
| | | TACGGCACGGGGCTGGATAACGCTACGG |
| | | TCGGCTTCCTTGGCATGGGCGCCATCGG |
| | | ACTGGCCATGGCTGATCGCTTGCAGGGA |
| | | TGGGGCGCGACCCTGCAGTACCACGCGG |
| | | CGAAGGCTCTGGATACACAAACCGAGC |
| | | AACGGCTCGGCCTGCGCCAGGTGGCGTG |
| | | CAGCGAACTCTTCGCCAGCTCGGACTTC |
| | | ATCCTGCTGGCGCTTCCCTTGAATGCCG |
| | | ATACCCTGCATCTGGTCAACGCCGAGCT |
| | | GCTTGCCCTCGTACGGCCGGGCGCTCTG |
| | | CTTGTAAACCCCTGTCGTGGCTCGGTAG |
| | | TGGATGAAGCCGCCGTGCTCGCGGCGCT |
| | | TGAGCGAGGCCAGCTCGGCGGGTATGCG |
| | | GCGGATGTATTCGAAATGGAAGACTGGG |
| | | CTCGCGCGGACCGGCCGCAGCAGATCGA |
| | | TCCTGCGCTGCTCGCGCATCCGAATACG |
| | | CTGTTCACTCCGCACATAGGGTCGGCAG |
| | | TGCGCGCGGTGCGCCTGGAGATTGAACG |
| | | TTGTGCAGCGCAGAACATCCTCCAGGCA |
| | | TTGGCAGGTGAGCGCCCAATCAACGCTG |
| | | TGAACCGTCTGCCCAAGGCCAATCCTGC |
| | | CGCAGAC |
| 56 | Malonate semialdehyde | ATGCCCTTAATCCGTATAGATCTTACCA |
| | decarboxylase (MSAD) | GTGATCGTTCGAGAGAGCAACGGCGGG |
| | from Coryneform sp. | CGATTGCTGATGCAGTCCATGACGCTTT |
| | FG41 (NCBI: | AGTAGAAGTTTTAGCGATTCCGGCTCGT |
| | MZ369160) plus TEV | GATCGCTTCCAGATACTGACTGCGCACG |
| | protease recognition site | ATCCCTCTGATATTATAGCCGAAGATGC |
| | and C-terminal His₆-tag | TGGACTTGGCTTTCAGCGGTCCCCCAGT |
| | | GTAGTCATCATACACGTCTTTACACAGG |
| | | CAGGTAGAACTATTGAAACGAAACAGA |
| | | GAGTATTTGCAGCGATAACAGAAAGTCT |
| | | GGCTCCAATCGGTGTTGCAGGATCTGAT |
| | | GTTTTTATCGCAATCACCGAAAATGCAC |
| | | CCCATGACTGGAGCTTTGGGTTTGGCAG |
| | | TGCACAATATGTCACGGGTGAACTTGCG |
| | | ATTCCAGCCACTGGTGCGGCTGAGAACC |
| | | TGTATTTTCAAGGCCTCGAGCACCACCA |
| | | CCACCACCAC |
| 57 | YdfG from Escherichia | ATGATCGTTTTAGTAACTGGAGCAACGG |
| | coli plus TEV protease | CAGGTTTTGGTGAATGCATTACTCGTCG |
| | recognition site and C- | TTTTATTCAACAAGGGCATAAAGTTATC |
| | terminal His₆-tag | GCCACTGGCCGTCGCCAGGAACGGTTGC |
| | | AGGAGTTAAAAGACGAACTGGGAGATA |
| | | ATCTGTATATCGCCCAACTGGACGTTCG |
| | | CAACCGCGCCGCTATTGAAGAGATGCTG |
| | | GCATCGCTTCCTGCCGAGTGGTGCAATA |
| | | TTGATATCCTGGTAAATAATGCCGGCCT |
| | | GGCGTTGGGCATGGAGCCTGCGCATAAA |
| | | GCCAGCGTTGAAGACTGGGAAACGATG |
| | | ATTGATACCAACAACAAAGGCCTGGTAT |
| | | ATATGACGCGCGCCGTCTTACCGGGTAT |
| | | GGTTGAACGTAATCATGGTCATATTATT |
| | | AACATTGGCTCAACGGCAGGTAGCTGGC |
| | | CGTATGCCGGTGGTAACGTTTACGGTGC |
| | | GACGAAAGCGTTTGTTCGTCAGTTTAGC |
| | | CTGAATCTGCGTACGGATCTGCATGGTA |
| | | CGGCGGTGCGCGTCACCGACATCGAACC |
| | | GGGTCTGGTGGGTGGTACCGAGTTTTCC |
| | | AATGTCCGCTTTAAAGGCGATGACGGTA |
| | | AAGCAGAAAAAACCTATCAAAATACCG |
| | | TTGCATTGACGCCAGAAGATGTCAGCGA |
| | | AGCCGTCTGGTGGGTGTCAACGCTGCCT |
| | | GCTCACGTCAATATCAATACCCTGGAAA |
| | | TGATGCCGGTTACCCAAAGCTATGCCGG |
| | | ACTGAATGTCCACCGTCAGGAGAACCTG |
| | | TATTTTCAAGGCCTCGAGCACCACCACC |
| | | ACCACCAC |
| 58 | MmsB from | ATGCGTATCGCATTCATCGGCCTGGGCA |
| | Pseudomonas putida | ACATGGGCGCGCCCATGGCCCGCAACCT |
| | KT2440 plus TEV | GATCAAGGCCGGGCATCAGCTGAACCTG |
| | protease recognition site | TTCGACCTGAACAAGGCCGTGCTGGCCG |
| | and C-terminal His₆-tag | AGCTGGCAGAACTGGGCGGGCAGATCA |
| | | GCCCCTCGCCCAAGGACGCGGCGGCCAA |
| | | CAGCGAGCTGGTGATCACCATGCTGCCG |
| | | GCCGCAGCCCATGTGCGTAGCGTGTACT |
| | | TGAACGAGGACGGCGTACTGGCCGGTAT |
| | | TCGTCCTGGCACGCCGACCGTTGACTGC |
| | | AGCACCATCGACCCGCAGACCGCACGTG |
| | | ACGTGTCCAAGGCCGCAGCGGCAAAGG |
| | | GCGTGGACATGGGGGATGCGCCGGTTTC |
| | | CGGTGGTACTGGCGGCGCGGCGGCCGGC |
| | | ACCCTGACGTTCATGGTCGGCGCCAGTA |
| | | CCGAGTTGTTCGCCAGCCTCAAGCCGGT |
| | | ACTGGAGCAGATGGGCCGCAACATCGTG |
| | | CACTGCGGGGAAGTCGGTACCGGCCAG |
| | | ATCGCCAAGATCTGCAACAACCTGCTGC |
| | | TCGGCATTTCGATGATCGGCGTGTCCGA |
| | | GGCCATGGCCCTGGGTAACGCGCTGGGT |
| | | ATCGATACCAAGGTGCTGGCCGGCATCA |
| | | TCAACAGTTCGACCGGGCGTTGCTGGAG |
| | | CTCGGACACCTACAACCCGTGGCCGGGC |
| | | ATCATCGAAACCGCACCTGCATCGCGTG |
| | | GCTACACCGGTGGCTTTGGCGCCGAACT |
| | | CATGCTCAAGGACCTGGGGTTGGCCACC |
| | | GAAGCGGCACGCCAGGCACACCAACCG |
| | | GTGATTCTCGGTGCCGTGGCCCAGCAGC |
| | | TGTACCAGGCCATGAGCCTGCGAGGCGA |
| | | GGGTGGCAAGGACTTCTCGGCCATCGTC |
| | | GAGGGTTATCGCAAGAAGGATGAGAAC |
| | | CTGTATTTTCAAGGCCTCGAGCACCACC |
| | | ACCACCACCAC |
| 59 | Cg10062 from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVYITEIPGSNMTEYGRLLM |
| | Escherichia coli plus | EPGEEEKWFNSLPEGLRERLTELEGSSEEN |
| | TEV protease | LYFQGLEHHHHHH |
| | recognition site and C- | |
| | terminal His₆-tag | |
| 60 | Cg10062(E114Q) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVYITEIPGSNMTQYGRLLM |
| | Escherichia coli plus | EPGEEEKWFNSLPEGLRERLTELEGSSEEN |
| | TEV protease | LYFQGLEHHHHHH |
| | recognition site and C- | |
| | terminal His₆-tag | |
| 61 | Cg10062(E114D) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVYITEIPGSNMTDYGRLLM |
| | Escherichia coli plus | EPGEEEKWFNSLPEGLRERLTELEGSSEEN |
| | TEV protease | LYFQGLEHHHHHH |
| | recognition site and C- | |
| | terminal His₆-tag | |
| 62 | Cg10062(E114N) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVYITEIPGSNMTNYGRLLM |
| | Escherichia coli plus | EPGEEEKWFNSLPEGLRERLTELEGSSEEN |
| | TEV protease | LYFQGLEHHHHHH |
| | recognition site and C- | |
| | terminal His₆-tag | |
| 63 | Cg10062(H28A) from | PTYTCWSQRIRISREAKQRIAEAITDAAHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVYITEIPGSNMTEYGRLLM |
| | Escherichia coli plus | EPGEEEKWFNSLPEGLRERLTELEGSSEEN |
| | TEV protease | LYFQGLEHHHHHH |
| | recognition site and C- | |
| | terminal His₆-tag | |
| 64 | Cg10062(R70A) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIASGRTEKQKEELLLRLTQEIA |
| | optimized for | LILGIPNEEVWVYITEIPGSNMTEYGRLLM |
| | Escherichia coli plus | EPGEEEKWFNSLPEGLRERLTELEGSSEEN |
| | TEV protease | LYFQGLEHHHHHH |
| | recognition site and C- | |
| | terminal His₆-tag | |
| 65 | Cg10062(R70K) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIKSGRTEKQKEELLLRLTQEIA |
| | optimized for | LILGIPNEEVWVYITEIPGSNMTEYGRLLM |
| | Escherichia coli plus | EPGEEEKWFNSLPEGLRERLTELEGSSEEN |
| | TEV protease | LYFQGLEHHHHHH |
| | recognition site and C- | |
| | terminal His₆-tag | |
| 66 | Cg10062(R73A) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGATEKQKEELLLRLTQEIA |
| | optimized for | LILGIPNEEVWVYITEIPGSNMTEYGRLLM |
| | Escherichia coli plus | EPGEEEKWFNSLPEGLRERLTELEGSSEEN |
| | TEV protease | LYFQGLEHHHHHH |
| | recognition site and C- | |
| | terminal His₆-tag | |
| 67 | Cg10062(R73K) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGKTEKQKEELLLRLTQEIA |
| | optimized for | LILGIPNEEVWVYITEIPGSNMTEYGRLLM |
| | Escherichia coli plus | EPGEEEKWFNSLPEGLRERLTELEGSSEEN |
| | TEV protease | LYFQGLEHHHHHH |
| | recognition site and C- | |
| | terminal His₆-tag | |
| 68 | Cg10062(Y103A) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVAITEIPGSNMTEYGRLLM |
| | Escherichia coli plus | EPGEEEKWFNSLPEGLRERLTELEGSSEEN |
| | TEV protease | LYFQGLEHHHHHH |
| | recognition site and C- | |
| | terminal His₆-tag | |
| 69 | Cg10062(Y103F) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVFITEIPGSNMTEYGRLLM |
| | Escherichia coli plus | EPGEEEKWFNSLPEGLRERLTELEGSSEEN |
| | TEV protease | LYFQGLEHHHHHH |
| | recognition site and C- | |
| | terminal His₆-tag | |
| 70 | Cg10062(E114A) from | PTYTCWSQRIRISREAKQRIAEAITDAHHE |
| | Corynebacterium | LAHAPKYLVQVIFNEVEPDSYFIAAQSASE |
| | glutamicum codon- | NHIWVQATIRSGRTEKQKEELLLRLTQEIA |
| | optimized for | LILGIPNEEVWVYITEIPGSNMTAYGRLLM |
| | Escherichia coli plus | EPGEEEKWFNSLPEGLRERLTELEGSSEEN |
| | TEV protease | LYFQGLEHHHHHH |
| | recognition site and C- | |
| | terminal His₆-tag | |
| 71 | Cg10062(E114D- | PTYTCWSQRIRISREAKQRIAEAITDAHHE |
| | Y103F) from | LAHAPKYLVQVIFNEVEPDSYFIAAQSASE |
| | Corynebacterium | NHIWVQATIRSGRTEKQKEELLLRLTQEIA |
| | glutamicum codon- | LILGIPNEEVWVFITEIPGSNMTDYGRLLM |
| | optimized for | EPGEEEKWFNSLPEGLRERLTELEGSSEEN |
| | Escherichia coli plus | LYFQGLEHHHHHH |
| | TEV protease | |
| | recognition site and C- | |
| | terminal His₆-tag | |
| 72 | Cis-CaaD from | PVYMVYVSQDRLTPSAKHAVAKAITDAH |
| | Coryneform sp. codon- | RGLTGTQHFLAQVNFQEQPAGNVFLGGV |
| | optimized for | QQGGDTIFVHGLHREGRSADLKGQLAQRI |
| | Escherichia coli plus | VDDVSVAAEIDRKHIWVYFGEMPAQQMV |
| | TEV protease | EYGRFLPQPGHEGEWFDNLSSDERAFMET |
| | recognition site and C- | NVDVSRTENLYFQGLEHHHHHH |
| | terminal His₆-tag | |
| 73 | Engineered phosphite | MGSSHHHHHHSSGLVPRGSHMLPKLVITH |
| | dehydrogenase (PTDH) | RVHEEILQLLAPHCELITNQTDSTLTREEIL |
| | from Pseudomonas | RRCRDAQAMMAFMPDRVDADFLQACPEL |
| | stutzeri plus N-terminal | RVIGCALKGFDNFDVDACTARGVWLTFV |
| | His₆-tag from pET-15b | PDLLTVPTAELAIGLAVGLGRHLRAADAF |
| | vector | VRSGKFRGWQPRFYGTGLDNATVGFLGM |
| | | GAIGLAMADRLQGWGATLQYHAAKALD |
| | | TQTEQRLGLRQVACSELFASSDFILLALPL |
| | | NADTLHLVNAELLALVRPGALLVNPCRGS |
| | | VVDEAAVLAALERGQLGGYAADVFEMED |
| | | WARADRPQQIDPALLAHPNTLFTPHIGSA |
| | | VRAVRLEIERCAAQNILQALAGERPINAVN |
| | | RLPKANPAAD |
| 74 | Malonate semialdehyde | PLIRIDLTSDRSREQRRAIADAVHDALVEV |
| | decarboxylase (MSAD) | LAIPARDRFQILTAHDPSDIIAEDAGLGFQR |
| | from Coryneform sp. | SPSVVIIHVFTQAGRTIETKQRVFAAITESL |
| | FG41 plus TEV | APIGVAGSDVFIAITENAPHDWSFGFGSAQ |
| | protease recognition site | YVTGELAIPATGAAENLYFQGLEHHHHHH |
| | and C-terminal His₆-tag | |
| 75 | YdfG from Escherichia | MIVLVTGATAGFGECITRRFIQQGHKVIAT |
| | coli plus TEV protease | GRRQERLQELKDELGDNLYIAQLDVRNRA |
| | recognition site and C- | AIEEMLASLPAEWCNIDILVNNAGLALGM |
| | terminal His₆-tag | EPAHKASVEDWETMIDTNNKGLVYMTRA |
| | | VLPGMVERNHGHIINIGSTAGSWPYAGGN |
| | | VYGATKAFVRQFSLNLRTDLHGTAVRVT |
| | | DIEPGLVGGTEFSNVRFKGDDGKAEKTYQ |
| | | NTVALTPEDVSEAVWWVSTLPAHVNINTL |
| | | EMMPVTQSYAGLNVHRQENLYFQGLEHH |
| | | HHHH |
| 76 | MmsB from | MRIAFIGLGNMGAPMARNLIKAGHQLNLF |
| | Pseudomonas putida | DLNKAVLAELAELGGQISPSPKDAAANSE |
| | KT2440 plus TEV | LVITMLPAAAHVRSVYLNEDGVLAGIRPG |
| | protease recognition site | TPTVDCSTIDPQTARDVSKAAAAKGVDM |
| | and C-terminal His₆-tag | GDAPVSGGTGGAAAGTLTFMVGASTELF |
| | | ASLKPVLEQMGRNIVHCGEVGTGQIAKIC |
| | | NNLLLGISMIGVSEAMALGNALGIDTKVL |
| | | AGIINSSTGRCWSSDTYNPWPGIIETAPASR |
| | | GYTGGFGAELMLKDLGLATEAARQAHQP |
| | | VILGAVAQQLYQAMSLRGEGGKDFSAIVE |
| | | GYRKKDENLYFQGLEHHHHHH |
| 77 | AS001 (F) Primer | CAACATGACCCAGTATGGCCGTC |
| 78 | AS002 (R) Primer | CTACCCGGGATTTCGGTA |
| 79 | AS003 (F) Primer | CAACATGACCGATTATGGCCGTC |
| 80 | AS012 (F) Primer | TACCGACGCGGCGCATGAACTGGCGCAC |
| | | G |
| 81 | AS013 (R) Primer | ATCGCTTCCGCGATGCGT |
| 82 | AS014 (F) Primer | AGCGACCATCGCGAGCGGCCGTAC |
| 83 | AS015 (R) Primer | TGAACCCAAATGTGGTTC |
| 84 | AS016 (F) Primer | AGCGACCATCAAAAGCGGCCGTA |
| 85 | AS017 (R) Primer | TGAACCCAAATGTGGTTCTC |
| 86 | AS018 (F) Primer | CCGTAGCGGCGCGACCGAAAAGC |
| 87 | AS019 (R) Primer | ATGGTCGCTTGAACCCAA |
| 88 | AS020 (F) Primer | CCGTAGCGGCAAAACCGAAAAGC |
| 89 | AS021 (F) Primer | AGTGTGGGTTGCGATTACCGAAATCCCG |
| | | GG |
| 90 | AS022 (R) Primer | TCCTCGTTCGGGATACCC |
| 91 | AS023 (F) Primer | AGTGTGGGTTTTTATTACCGAAATCCC |
| 92 | AS024 (F) Primer | CAACATGACCGCGTATGGCCGTCTG |
| 93 | AS026 (F) Primer | CAACATGACCAACTATGGCCGTCTG |

What is claimed is:

1. A method of making 3-hydroxypropionic acid (3-HP) or an anion or salt thereof, the method comprising
hydrating acetylenecarboxylic acid (ACA) or an anion or salt thereof by reacting the ACA or the anion or salt thereof with an ACA-hydrating enzyme to form a reaction product comprising malonic semialdehyde (MSA) or an anion or salt thereof; and
reacting the reaction product comprising MSA or the anion or salt thereof with a pair of oxidoreductases in an oxidation-reduction (redox) reaction to produce 3-HP or the anion or salt thereof; wherein the pair of oxidoreductases cycle a cofactor, such as NADPH or NADH.

2. The method of claim 1, wherein the ACA-hydrating enzyme is a tautomerase.

3. The method of claim 2, wherein the tautomerase is substantially free of decarboxylase activity.

4. The method of claim 2, wherein the tautomerase comprises Cg10062 (wild-type) or a variant thereof capable of hydrating ACA or the anion or salt thereof; or cis-CaaD or a variant thereof capable of hydrating ACA or the anion or salt thereof.

5. The method of claim 4, wherein the Cg10062 or variant thereof has at least 85% sequence identity to SEQ ID NO: 21 or SEQ ID NO: 59.

6. The method of claim 4, wherein the cis-CaaD or variant thereof has at least 85% sequence identity to SEQ ID NO: 34 or SEQ ID NO: 72.

7. The method of claim 4, wherein the variant of Cg10062 comprises at least one mutation at an amino acid position corresponding to amino acid position 28, 70, 73, 103, and 114.

8. The method of claim 7, wherein the variant of Cg10062 has one or more mutations selected from the group consisting of H28A, R70A, R70K, R73A, R73K, Y103A, Y103F, E114A, E114D, E114N, and E114Q.

9. The method of claim 8, wherein the variant of Cg10062 has the E114N mutation.

10. The method of claim 1, wherein the pair of oxidoreductases that cycle the cofactor are YdfG and PTDH or MmsB and SH.

11. The method of claim 10, wherein the YdfG has at least 85% sequence identity to SEQ ID NO: 37 or 75, the PTDH has at least 85% sequence identity to SEQ ID NO: 35 or 73, and the MmsB has at least 85% sequence identity to SEQ ID NO: 38 or 76.

12. The method of claim 1, where the reaction product comprising MSA or the anion or salt thereof comprises about 95% or more MSA or the anion or salt thereof and about 5% or less of other reaction products and is substantially free of acetaldehyde and CO₂.

13. The method of claim 1, further comprising synthesizing the ACA or the anion or salt thereof by dehydrodimerization of CH₄ to produce acetylene and reacting the acetylene with CO₂ to produce the ACA or the anion or salt thereof.

14. A method of making 3-HP or an anion or salt thereof, the method comprising adding ACA or an anion or salt thereof to a cell culture comprising a recombinant microorganism and a carbon source, wherein the recombinant microorganism is genetically engineered to express an ACA-hydrating enzyme and an oxidoreductase.

15. The method of claim 14, wherein the ACA-hydrating enzyme is a tautomerase.

16. The method of claim 15, wherein the tautomerase is substantially free of decarboxylase activity.

17. The method of claim 15, wherein the tautomerase comprises Cg10062 (wild-type) or a variant thereof capable of hydrating ACA or the anion or salt thereof; or cis-CaaD or a variant thereof capable of hydrating ACA or the anion or salt thereof.

18. The method of claim 17, wherein the Cg10062 or variant thereof has at least 85% sequence identity to SEQ ID NO: 21 or SEQ ID NO: 59.

19. The method of claim 17, wherein the cis-CaaD or variant thereof has at least 85% sequence identity to SEQ ID NO: 34 or SEQ ID NO: 72.

20. The method of claim 17, wherein the variant of Cg10062 comprises at least one mutation at an amino acid position corresponding to amino acid position 28, 70, 73, 103, and 114.

21. The method of claim 20, wherein the variant of Cg10062 has one or more mutations selected from the group consisting of H28A, R70A, R70K, R73A, R73K, Y103A, Y103F, E114A, E114D, E114N, and E114Q.

22. The method of claim 21, wherein the variant of Cg10062 has the E114N mutation.

23. The method of claim 14, wherein the oxidoreductase is YdfG.

24. The method of claim 23, wherein the YdfG has at least 85% sequence identity to SEQ ID NO: 37 or 75.

25. The method of claim 14, further comprising isolating the 3-HP or the anion or salt thereof from the cell culture.

26. A composition produced by reacting ACA or an anion or salt thereof with an ACA-hydrating enzyme, wherein the composition comprises at least 95% MSA or an anion or salt thereof and less than 5% acetaldehyde and CO₂.

27. A non-naturally occurring variant tautomerase comprising an amino acid sequence of SEQ ID NO: 24 or SEQ ID NO: 62.

28. A vector comprising a nucleotide sequence encoding a variant tautomerase comprising an amino acid sequence of SEQ ID NO: 24 or SEQ ID NO: 62.

29. A recombinant cell genetically engineered to express a variant tautomerase comprising the amino acid sequence of SEQ ID NO: 24 or SEQ ID NO: 62.

30. The recombinant cell of claim 29 genetically engineered to additionally express one or more oxidoreductases comprising an amino acid sequence having 85% sequence identity to SEQ ID NO: 35, 37, 38, 73, 75, or 76.


SEQUENCE LISTING (93 sequences)

SEQ ID NO: 1 - DNA, 447 bp, synthetic construct
  misc_feature 1..447: note = Cg10062 (NCBI: MZ369159) from Corynebacterium glutamicum codon-optimized for Escherichia coli
  source 1..447: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttacattaccgaaatcccgggtagcaacatgaccgaatatggccgtctgctgatggagccgggcgaggaagagaaatggttcaacagcctgccggagggcctgcgtgagcgtctgaccgaactggagggtagcagcgaa

SEQ ID NO: 2 - DNA, 447 bp, synthetic construct
  misc_feature 1..447: note = Cg10062(E114Q) from Corynebacterium glutamicum codon-optimized for Escherichia coli
  source 1..447: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttacattaccgaaatcccgggtagcaacatgacccagtatggccgtctgctgatggagccgggcgaggaagagaaatggttcaacagcctgccggagggcctgcgtgagcgtctgaccgaactggagggtagcagcgaa

SEQ ID NO: 3 - DNA, 447 bp, synthetic construct
  misc_feature 1..447: note = Cg10062(E114D) from Corynebacterium glutamicum codon-optimized for Escherichia coli
  source 1..447: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttacattaccgaaatcccgggtagcaacatgaccgattatggccgtctgctgatggagccgggcgaggaagagaaatggttcaacagcctgccggagggcctgcgtgagcgtctgaccgaactggagggtagcagcgaa

SEQ ID NO: 4 - DNA, 447 bp, synthetic construct
  misc_feature 1..447: note = Cg10062(E114N) from Corynebacterium glutamicum codon-optimized for Escherichia coli
  source 1..447: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttacattaccgaaatcccgggtagcaacatgaccaactatggccgtctgctgatggagccgggcgaggaagagaaatggttcaacagcctgccggagggcctgcgtgagcgtctgaccgaactggagggtagcagcgaa

SEQ ID NO: 5 - DNA, 447 bp, synthetic construct
  misc_feature 1..447: note = Cg10062(H28A) from Corynebacterium glutamicum codon-optimized for Escherichia coli
  source 1..447: mol_type = other DNA
atgccgacctacacctgctggagccaacgcattcgtattagccgtgaagcgaagcaacgcatcgcggaagcgattaccgacgcggcgcatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttacattaccgaaatcccgggtagcaacatgaccgaatatggccgtctgctgatggagccgggcgaggaagagaaatggttcaacagcctgccggagggcctgcgtgagcgtctgaccgaactggagggtagcagcgaa

SEQ ID NO: 6 - DNA, 447 bp, synthetic construct
  misc_feature 1..447: note = Cg10062(R70A) from Corynebacterium glutamicum codon-optimized for Escherichia coli
  source 1..447: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatcgcgagcggccgtaccgaaaagcagaaagaggaactgctgctgcgtctgacccaagagatcgcgctgattctgggtatcccgaacgaggaagtgtgggtttacattaccgaaatcccgggtagcaacatgaccgaatatggccgtctgctgatggagccgggcgaggaagagaaatggttcaacagcctgccggagggcctgcgtgagcgtctgaccgaactggagggtagcagcgaa

SEQ ID NO: 7 - DNA, 447 bp, synthetic construct
  misc_feature 1..447: note = Cg10062(R70K) from Corynebacterium glutamicum codon-optimized for Escherichia coli
  source 1..447: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatcaaaagcggccgtaccgaaaagcagaaagaggaactgctgctgcgtctgacccaagagatcgcgctgattctgggtatcccgaacgaggaagtgtgggtttacattaccgaaatcccgggtagcaacatgaccgaatatggccgtctgctgatggagccgggcgaggaagagaaatggttcaacagcctgccggagggcctgcgtgagcgtctgaccgaactggagggtagcagcgaa

SEQ ID NO: 8 - DNA, 447 bp, synthetic construct
  misc_feature 1..447: note = Cg10062(R73A) from Corynebacterium glutamicum codon-optimized for Escherichia coli
  source 1..447: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggcgcgaccgaaaagcagaaagaggaactgctgctgcgtctgacccaagagatcgcgctgattctgggtatcccgaacgaggaagtgtgggtttacattaccgaaatcccgggtagcaacatgaccgaatatggccgtctgctgatggagccgggcgaggaagagaaatggttcaacagcctgccggagggcctgcgtgagcgtctgaccgaactggagggtagcagcgaa

SEQ ID NO: 9 - DNA, 447 bp, synthetic construct
  misc_feature 1..447: note = Cg10062(R73K) from Corynebacterium glutamicum codon-optimized for Escherichia coli
  source 1..447: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggcaaaaccgaaaagcagaaagaggaactgctgctgcgtctgacccaagagatcgcgctgattctgggtatcccgaacgaggaagtgtgggtttacattaccgaaatcccgggtagcaacatgaccgaatatggccgtctgctgatggagccgggcgaggaagagaaatggttcaacagcctgccggagggcctgcgtgagcgtctgaccgaactggagggtagcagcgaa

SEQ ID NO: 10 - DNA, 447 bp, synthetic construct
  misc_feature 1..447: note = Cg10062(Y103A) from Corynebacterium glutamicum codon-optimized for Escherichia coli
  source 1..447: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggttgcgattaccgaaatcccgggtagcaacatgaccgaatatggccgtctgctgatggagccgggcgaggaagagaaatggttcaacagcctgccggagggcctgcgtgagcgtctgaccgaactggagggtagcagcgaa

SEQ ID NO: 11 - DNA, 447 bp, synthetic construct
  misc_feature 1..447: note = Cg10062(Y103F) from Corynebacterium glutamicum codon-optimized for Escherichia coli
  source 1..447: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttttattaccgaaatcccgggtagcaacatgaccgaatatggccgtctgctgatggagccgggcgaggaagagaaatggttcaacagcctgccggagggcctgcgtgagcgtctgaccgaactggagggtagcagcgaa

SEQ ID NO: 12 - DNA, 447 bp, synthetic construct
  misc_feature 1..447: note = Cg10062(E114A) from Corynebacterium glutamicum codon-optimized for Escherichia coli
  source 1..447: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttacattaccgaaatcccgggtagcaacatgaccgcgtatggccgtctgctgatggagccgggcgaggaagagaaatggttcaacagcctgccggagggcctgcgtgagcgtctgaccgaactggagggtagcagcgaa

SEQ ID NO: 13 - DNA, 447 bp, synthetic construct
  misc_feature 1..447: note = Cg10062(E114D-Y103F) from Corynebacterium glutamicum codon-optimized for Escherichia coli
  source 1..447: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttttattaccgaaatcccgggtagcaacatgaccgattatggccgtctgctgatggagccgggcgaggaagagaaatggttcaacagcctgccggagggcctgcgtgagcgtctgaccgaactggagggtagcagcgaa

SEQ ID NO: 14 - DNA, 450 bp, synthetic construct
  misc_feature 1..450: note = Cis-CaaD from Coryneform sp. codon-optimized for expression in Escherichia coli
  source 1..450: mol_type = other DNA
atgccggtttacatggtttacgttagccaggaccgtctgaccccgagcgcgaagcacgcggttgcgaaggcgattaccgatgcgcaccgtggtctgaccggcacccagcacttcctggcgcaagtgaactttcaggagcaaccggcgggtaacgtgttcctgggtggcgttcagcaaggtggcgacaccatctttgttcatggtctgcaccgtgagggccgtagcgcggatctgaagggccagctggcgcaacgtattgttgacgatgtgagcgttgcggcggaaatcgaccgtaaacacatttgggtgtacttcggcgagatgccggcgcagcaaatggttgaatatggccgtttcctgccgcagccgggtcatgagggtgaatggtttgacaacctgagcagcgatgaacgtgcgtttatggagaccaatgttgatgtgagccgtacc

SEQ ID NO: 15 - DNA, 1008 bp, synthetic construct
  misc_feature 1..1008: note = Engineered phosphite dehydrogenase (PTDH) from Pseudomonas stutzeri
  source 1..1008: mol_type = other DNA
atgctgccgaaactcgttataactcaccgagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttccggggctggcaaccacggttctacggcacggggctggataacgctacggtcggcttccttggcatgggcgccatcggactggccatggctgatcgcttgcagggatggggcgcgaccctgcagtaccacgcggcgaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccaatcctgccgcagac

SEQ ID NO: 16 - DNA, 411 bp, Coryneform sp.
  source 1..411: mol_type = unassigned DNA; strain = FG41
atgcccttaatccgtatagatcttaccagtgatcgttcgagagagcaacggcgggcgattgctgatgcagtccatgacgctttagtagaagttttagcgattccggctcgtgatcgcttccagatactgactgcgcacgatccctctgatattatagccgaagatgctggacttggctttcagcggtcccccagtgtagtcatcatacacgtctttacacaggcaggtagaactattgaaacgaaacagagagtatttgcagcgataacagaaagtctggctccaatcggtgttgcaggatctgatgtttttatcgcaatcaccgaaaatgcaccccatgactggagctttgggtttggcagtgcacaatatgtcacgggtgaacttgcgattccagccactggtgcggct

SEQ ID NO: 17 - DNA, 744 bp, Escherichia coli
  source 1..744: mol_type = unassigned DNA
atgatcgttttagtaactggagcaacggcaggttttggtgaatgcattactcgtcgttttattcaacaagggcataaagttatcgccactggccgtcgccaggaacggttgcaggagttaaaagacgaactgggagataatctgtatatcgcccaactggacgttcgcaaccgcgccgctattgaagagatgctggcatcgcttcctgccgagtggtgcaatattgatatcctggtaaataatgccggcctggcgttgggcatggagcctgcgcataaagccagcgttgaagactgggaaacgatgattgataccaacaacaaaggcctggtatatatgacgcgcgccgtcttaccgggtatggttgaacgtaatcatggtcatattattaacattggctcaacggcaggtagctggccgtatgccggtggtaacgtttacggtgcgacgaaagcgtttgttcgtcagtttagcctgaatctgcgtacggatctgcatggtacggcggtgcgcgtcaccgacatcgaaccgggtctggtgggtggtaccgagttttccaatgtccgctttaaaggcgatgacggtaaagcagaaaaaacctatcaaaataccgttgcattgacgccagaagatgtcagcgaagccgtctggtgggtgtcaacgctgcctgctcacgtcaatatcaataccctggaaatgatgccggttacccaaagctatgccggactgaatgtccaccgtcag

SEQ ID NO: 18 - DNA, 885 bp, Pseudomonas putida
  source 1..885: mol_type = unassigned DNA; strain = KT2440
atgcgtatcgcattcatcggcctgggcaacatgggcgcgcccatggcccgcaacctgatcaaggccgggcatcagctgaacctgttcgacctgaacaaggccgtgctggccgagctggcagaactgggcgggcagatcagcccctcgcccaaggacgcggcggccaacagcgagctggtgatcaccatgctgccggccgcagcccatgtgcgtagcgtgtacttgaacgaggacggcgtactggccggtattcgtcctggcacgccgaccgttgactgcagcaccatcgacccgcagaccgcacgtgacgtgtccaaggccgcagcggcaaagggcgtggacatgggggatgcgccggtttccggtggtactggcggcgcggcggccggcaccctgacgttcatggtcggcgccagtaccgagttgttcgccagcctcaagccggtactggagcagatgggccgcaacatcgtgcactgcggggaagtcggtaccggccagatcgccaagatctgcaacaacctgctgctcggcatttcgatgatcggcgtgtccgaggccatggccctgggtaacgcgctgggtatcgataccaaggtgctggccggcatcatcaacagttcgaccgggcgttgctggagctcggacacctacaacccgtggccgggcatcatcgaaaccgcacctgcatcgcgtggctacaccggtggctttggcgccgaactcatgctcaaggacctggggttggccaccgaagcggcacgccaggcacaccaaccggtgattctcggtgccgtggcccagcagctgtaccaggccatgagcctgcgaggcgagggtggcaaggacttctcggccatcgtcgagggttatcgcaagaaggat

SEQ ID NO: 19 - DNA, 60 bp, synthetic construct
  misc_feature 1..60: note = PTDH N-terminal His6-tag from pET-15b vector
  source 1..60: mol_type = other DNA
atgggcagcagccatcatcatcatcatcacagcagcggcctggtgccgcgcggcagccat

SEQ ID NO: 20 - DNA, 45 bp, synthetic construct
  misc_feature 1..45: note = TEV protease recognition site and C-terminal His6-tag
  source 1..45: mol_type = other DNA
gagaacctgtattttcaaggcctcgagcaccaccaccaccaccac

SEQ ID NO: 21 - protein, 148 aa, synthetic construct
  REGION 1..148: note = Cg10062 from Corynebacterium glutamicum codon-optimized for Escherichia coli
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVYITEIPGSNMTEYGRLLMEPGEEEKWFNSLPEGLRERLTELEGSSE

SEQ ID NO: 22 - protein, 148 aa, synthetic construct
  REGION 1..148: note = Cg10062(E114Q) from Corynebacterium glutamicum codon-optimized for Escherichia coli
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVYITEIPGSNMTQYGRLLMEPGEEEKWFNSLPEGLRERLTELEGSSE

SEQ ID NO: 23 - protein, 148 aa, synthetic construct
  REGION 1..148: note = Cg10062(E114D) from Corynebacterium glutamicum codon-optimized for Escherichia coli
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVYITEIPGSNMTDYGRLLMEPGEEEKWFNSLPEGLRERLTELEGSSE

SEQ ID NO: 24 - protein, 148 aa, synthetic construct
  REGION 1..148: note = Cg10062(E114N) from Corynebacterium glutamicum codon-optimized for Escherichia coli
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVYITEIPGSNMTNYGRLLMEPGEEEKWFNSLPEGLRERLTELEGSSE

SEQ ID NO: 25 - protein, 148 aa, synthetic construct
  REGION 1..148: note = Cg10062(H28A) from Corynebacterium glutamicum codon-optimized for Escherichia coli
PTYTCWSQRIRISREAKQRIAEAITDAAHELAHAPKYLVQVIFNEVEPDSYFIAAQSASENHIWVQATIRSGRTEKQKEELLLRLTQEIALILGIPNEEVWVYITEIPGSNMTEYGRLLMEPGEEEKWFNSLPEGLRERLTELEGSSE

SEQ ID NO: 26 - protein, 148 aa, synthetic construct
  REGION 1..148: note = Cg10062(R70A) from Corynebacterium glutamicum codon-optimized for Escherichia coli
PTYTCWSQRIRISREAKQRIAEAITDAHHELAHAPKYLVQVIFNEVEPDSYFIAAQSASENHIWVQATIASGRTEKQKEELLLRLTQEIALILGIPNEEVWVYITEIPGSNMTEYGRLLMEPGEEEKWFNSLPEGLRERLTELEGSSE

SEQ ID NO: 27 - protein, 148 aa, synthetic construct
  REGION 1..148: note = Cg10062(R70K) from Corynebacterium glutamicum codon-optimized for Escherichia coli
PTYTCWSQRIRISREAKQRIAEAITDAHHELAHAPKYLVQVIFNEVEPDSYFIAAQSASENHIWVQATIKSGRTEKQKEELLLRLTQEIALILGIPNEEVWVYITEIPGSNMTEYGRLLMEPGEEEKWFNSLPEGLRERLTELEGSSE

SEQ ID NO: 28 - protein, 148 aa, synthetic construct
  REGION 1..148: note = Cg10062(R73A) from Corynebacterium glutamicum codon-optimized for Escherichia coli
PTYTCWSQRIRISREAKQRIAEAITDAHHELAHAPKYLVQVIFNEVEPDSYFIAAQSASENHIWVQATIRSGATEKQKEELLLRLTQEIALILGIPNEEVWVYITEIPGSNMTEYGRLLMEPGEEEKWFNSLPEGLRERLTELEGSSE

SEQ ID NO: 29 - protein, 148 aa, synthetic construct
  REGION 1..148: note = Cg10062(R73K) from Corynebacterium glutamicum codon-optimized for Escherichia coli
PTYTCWSQRIRISREAKQRIAEAITDAHHELAHAPKYLVQVIFNEVEPDSYFIAAQSASENHIWVQATIRSGKTEKQKEELLLRLTQEIALILGIPNEEVWVYITEIPGSNMTEYGRLLMEPGEEEKWFNSLPEGLRERLTELEGSSE

SEQ ID NO: 30 - protein, 148 aa, synthetic construct
  REGION 1..148: note = Cg10062(Y103A) from Corynebacterium glutamicum codon-optimized for Escherichia coli
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVAITEIPGSNMTEYGRLLMEPGEEEKWFNSLPEGLRERLTELEGSSE

SEQ ID NO: 31 - protein, 148 aa, synthetic construct
  REGION 1..148: note = Cg10062(Y103F) from Corynebacterium glutamicum codon-optimized for Escherichia coli
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVFITEIPGSNMTEYGRLLMEPGEEEKWFNSLPEGLRERLTELEGSSE

SEQ ID NO: 32 - protein, 148 aa, synthetic construct
  REGION 1..148: note = Cg10062(E114A) from Corynebacterium glutamicum codon-optimized for Escherichia coli
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVYITEIPGSNMTAYGRLLMEPGEEEKWFNSLPEGLRERLTELEGSSE

SEQ ID NO: 33 - protein, 148 aa, synthetic construct
  REGION 1..148: note = Cg10062(E114D-Y103F) from Corynebacterium glutamicum codon-optimized for Escherichia coli
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVFITEIPGSNMTDYGRLLMEPGEEEKWFNSLPEGLRERLTELEGSSE

SEQ ID NO: 34 - protein, 149 aa, synthetic construct
  REGION 1..149: note = Cis-CaaD from Coryneform sp. codon-optimized for expression in Escherichia coli
PVYMVYVSQDRLTPSAKHAVAKAITDAHRGLTGTQHFLAQVNFQEQPAGNVFLGGVQQGGDTIFVHGLHREGRSADLKGQLAQRIVDDVSVAAEIDRKHIWVYFGEMPAQQMVEYGRFLPQPGHEGEWFDNLSSDERAFMETNVDVSRT

SEQ ID NO: 35 - protein, 336 aa, synthetic construct
  REGION 1..336: note = Engineered phosphite dehydrogenase (PTDH) from Pseudomonas stutzeri
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGAIGLAMADRLQGWGATLQYHAAKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKANPAAD

SEQ ID NO: 36 - protein, 136 aa, Coryneform sp.
  source 1..136: note = strain= FG41
PLIRIDLTSDRSREQRRAIADAVHDALVEVLAIPARDRFQILTAHDPSDIIAEDAGLGFQRSPSVVIIHVFTQAGRTIETKQRVFAAITESLAPIGVAGSDVFIAITENAPHDWSFGFGSAQYVTGELAIPATGAA

SEQ ID NO: 37 - protein, 248 aa, Escherichia coli
MIVLVTGATAGFGECITRRFIQQGHKVIATGRRQERLQELKDELGDNLYIAQLDVRNRAAIEEMLASLPAEWCNIDILVNNAGLALGMEPAHKASVEDWETMIDTNNKGLVYMTRAVLPGMVERNHGHIINIGSTAGSWPYAGGNVYGATKAFVRQFSLNLRTDLHGTAVRVTDIEPGLVGGTEFSNVRFKGDDGKAEKTYQNTVALTPEDVSEAVWWVSTLPAHVNINTLEMMPVTQSYAGLNVHRQ

SEQ ID NO: 38 - protein, 295 aa, Pseudomonas putida
  source 1..295: note = strain = KT2440
MRIAFIGLGNMGAPMARNLIKAGHQLNLFDLNKAVLAELAELGGQISPSPKDAAANSELVITMLPAAAHVRSVYLNEDGVLAGIRPGTPTVDCSTIDPQTARDVSKAAAAKGVDMGDAPVSGGTGGAAAGTLTFMVGASTELFASLKPVLEQMGRNIVHCGEVGTGQIAKICNNLLLGISMIGVSEAMALGNALGIDTKVLAGIINSSTGRCWSSDTYNPWPGIIETAPASRGYTGGFGAELMLKDLGLATEAARQAHQPVILGAVAQQLYQAMSLRGEGGKDFSAIVEGYRKKD

SEQ ID NO: 39 - protein, 20 aa, synthetic construct
  REGION 1..20: note = PTDH N-terminal His6-tag from pET-15b vector
MGSSHHHHHHSSGLVPRGSH

SEQ ID NO: 40 - protein, 15 aa, synthetic construct
  REGION 1..15: note = TEV protease recognition site and C-terminal His6-tag
ENLYFQGLEHHHHHH

SEQ ID NO: 41 - DNA, 492 bp, synthetic construct
  misc_feature 1..492: note = Cg10062 (NCBI: MZ369159) from Corynebacterium glutamicum codon-optimized for Escherichia coli plus TEV protease recognition site and C-terminal His6-tag
  source 1..492: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttacattaccgaaatcccgggtagcaacatgaccgaatatggccgtctgctgatggagccgggcgaggaagagaaatggttcaacagcctgccggagggcctgcgtgagcgtctgaccgaactggagggtagcagcgaagagaacctgtattttcaaggcctcgagcaccaccaccaccaccac

SEQ ID NO: 42 - DNA, 492 bp, synthetic construct
  misc_feature 1..492: note = Cg10062(E114Q) from Corynebacterium glutamicum codon-optimized for Escherichia coli plus TEV protease recognition site and C-terminal His6-tag
  source 1..492: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttacattaccgaaatcccgggtagcaacatgacccagtatggccgtctgctgatggagccgggcgaggaagagaaatggttcaacagcctgccggagggcctgcgtgagcgtctgaccgaactggagggtagcagcgaagagaacctgtattttcaaggcctcgagcaccaccaccaccaccac

SEQ ID NO: 43 - DNA, 492 bp, synthetic construct
  misc_feature 1..492: note = Cg10062(E114D) from Corynebacterium glutamicum codon-optimized for Escherichia coli plus TEV protease recognition site and C-terminal His6-tag
  source 1..492: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttacattaccgaaatcccgggtagcaacatgaccgattatggccgtctgctgatggagccgggcgaggaagagaaatggttcaacagcctgccggagggcctgcgtgagcgtctgaccgaactggagggtagcagcgaagagaacctgtattttcaaggcctcgagcaccaccaccaccaccac

SEQ ID NO: 44 - DNA, 492 bp, synthetic construct
  misc_feature 1..492: note = Cg10062(E114N) from Corynebacterium glutamicum codon-optimized for Escherichia coli plus TEV protease recognition site and C-terminal His6-tag
  source 1..492: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttacattaccgaaatcccgggtagcaacatgaccaactatggccgtctgctgatggagccgggcgaggaagagaaatggttcaacagcctgccggagggcctgcgtgagcgtctgaccgaactggagggtagcagcgaagagaacctgtattttcaaggcctcgagcaccaccaccaccaccac

SEQ ID NO: 45 - DNA, 492 bp, synthetic construct
  misc_feature 1..492: note = Cg10062(H28A) from Corynebacterium glutamicum codon-optimized for Escherichia coli plus TEV protease recognition site and C-terminal His6-tag
  source 1..492: mol_type = other DNA
atgccgacctacacctgctggagccaacgcattcgtattagccgtgaagcgaagcaacgcatcgcggaagcgattaccgacgcggcgcatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttacattaccgaaatcccgggtagcaacatgaccgaatatggccgtctgctgatggagccgggcgaggaagagaaatggttcaacagcctgccggagggcctgcgtgagcgtctgaccgaactggagggtagcagcgaagagaacctgtattttcaaggcctcgagcaccaccaccaccaccac

SEQ ID NO: 46 - DNA, 492 bp, synthetic construct
  misc_feature 1..492: note = Cg10062(R70A) from Corynebacterium glutamicum codon-optimized for Escherichia coli plus TEV protease recognition site and C-terminal His6-tag
  source 1..492: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatcgcgagcggccgtaccgaaaagcagaaagaggaactgctgctgcgtctgacccaagagatcgcgctgattctgggtatcccgaacgaggaagtgtgggtttacattaccgaaatcccgggtagcaacatgaccgaatatggccgtctgctgatggagccgggcgaggaagagaaatggttcaacagcctgccggagggcctgcgtgagcgtctgaccgaactggagggtagcagcgaagagaacctgtattttcaaggcctcgagcaccaccaccaccaccac

SEQ ID NO: 47 - DNA, 492 bp, synthetic construct
  misc_feature 1..492: note = Cg10062(R70K) from Corynebacterium glutamicum codon-optimized for Escherichia coli plus TEV protease recognition site and C-terminal His6-tag
  source 1..492: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatcaaaagcggccgtaccgaaaagcagaaagaggaactgctgctgcgtctgacccaagagatcgcgctgattctgggtatcccgaacgaggaagtgtgggtttacattaccgaaatcccgggtagcaacatgaccgaatatggccgtctgctgatggagccgggcgaggaagagaaatggttcaacagcctgccggagggcctgcgtgagcgtctgaccgaactggagggtagcagcgaagagaacctgtattttcaaggcctcgagcaccaccaccaccaccac

SEQ ID NO: 48 - DNA, 492 bp, synthetic construct
  misc_feature 1..492: note = Cg10062(R73A) from Corynebacterium glutamicum codon-optimized for Escherichia coli plus TEV protease recognition site and C-terminal His6-tag
  source 1..492: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggcgcgaccgaaaagcagaaagaggaactgctgctgcgtctgacccaagagatcgcgctgattctgggtatcccgaacgaggaagtgtgggtttacattaccgaaatcccgggtagcaacatgaccgaatatggccgtctgctgatggagccgggcgaggaagagaaatggttcaacagcctgccggagggcctgcgtgagcgtctgaccgaactggagggtagcagcgaagagaacctgtattttcaaggcctcgagcaccaccaccaccaccac

SEQ ID NO: 49 - DNA, 492 bp, synthetic construct
  misc_feature 1..492: note = Cg10062(R73K) from Corynebacterium glutamicum codon-optimized for Escherichia coli plus TEV protease recognition site and C-terminal His6-tag
  source 1..492: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggcaaaaccgaaaagcagaaagaggaactgctgctgcgtctgacccaagagatcgcgctgattctgggtatcccgaacgaggaagtgtgggtttacattaccgaaatcccgggtagcaacatgaccgaatatggccgtctgctgatggagccgggcgaggaagagaaatggttcaacagcctgccggagggcctgcgtgagcgtctgaccgaactggagggtagcagcgaagagaacctgtattttcaaggcctcgagcaccaccaccaccaccac

SEQ ID NO: 50 - DNA, 492 bp, synthetic construct
  misc_feature 1..492: note = Cg10062(Y103A) from Corynebacterium glutamicum codon-optimized for Escherichia coli plus TEV protease recognition site and C-terminal His6-tag
  source 1..492: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggttgcgattaccgaaatcccgggtagcaacatgaccgaatatggccgtctgctgatggagccgggcgaggaagagaaatggttcaacagcctgccggagggcctgcgtgagcgtctgaccgaactggagggtagcagcgaagagaacctgtattttcaaggcctcgagcaccaccaccaccaccac

SEQ ID NO: 51 - DNA, 492 bp, synthetic construct
  misc_feature 1..492: note = Cg10062(Y103F) from Corynebacterium glutamicum codon-optimized for Escherichia coli plus TEV protease recognition site and C-terminal His6-tag
  source 1..492: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttttattaccgaaatcccgggtagcaacatgaccgaatatggccgtctgctgatggagccgggcgaggaagagaaatggttcaacagcctgccggagggcctgcgtgagcgtctgaccgaactggagggtagcagcgaagagaacctgtattttcaaggcctcgagcaccaccaccaccaccac

SEQ ID NO: 52 - DNA, 492 bp, synthetic construct
  misc_feature 1..492: note = Cg10062(E114A) from Corynebacterium glutamicum codon-optimized for Escherichia coli plus TEV protease recognition site and C-terminal His6-tag
  source 1..492: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttacattaccgaaatcccgggtagcaacatgaccgcgtatggccgtctgctgatggagccgggcgaggaagagaaatggttcaacagcctgccggagggcctgcgtgagcgtctgaccgaactggagggtagcagcgaagagaacctgtattttcaaggcctcgagcaccaccaccaccaccac

SEQ ID NO: 53 - DNA, 492 bp, synthetic construct
  misc_feature 1..492: note = Cg10062(E114D-Y103F) from Corynebacterium glutamicum codon-optimized for Escherichia coli plus TEV protease recognition site and C-terminal His6-tag
  source 1..492: mol_type = other DNA
atgccgacctacacctgctggagccaacgcattcgtattagccgtgaagcgaagcaacgcatcgcggaagcgattaccgacgcgcaccatgaactggcgcacgcgccgaagtacctggtgcaggttattttcaacgaagtggagccggacagctattttatcgcggcgcagagcgcgagcgagaaccacatttgggttcaagcgaccatccgtagcggccgtaccgaaaagcagaaagaggaactgctgctgcgtctgacccaagagatcgcgctgattctgggtatcccgaacgaggaagtgtgggtttttattaccgaaatcccgggtagcaacatgaccgattatggccgtctgctgatggagccgggcgaggaagagaaatggttcaacagcctgccggagggcctgcgtgagcgtctgaccgaactggagggtagcagcgaagagaacctgtattttcaaggcctcgagcaccaccaccaccaccac

SEQ ID NO: 54 - DNA, 495 bp, synthetic construct
  misc_feature 1..495: note = Cis-CaaD from Coryneform sp. codon-optimized for Escherichia coli plus TEV protease recognition site and C-terminal His6-tag
  source 1..495: mol_type = other DNA
atgccggtttacatggtttacgttagccaggaccgtctgaccccgagcgcgaagcacgcggttgcgaaggcgattaccgatgcgcaccgtggtctgaccggcacccagcacttcctggcgcaagtgaactttcaggagcaaccggcgggtaacgtgttcctgggtggcgttcagcaaggtggcgacaccatctttgttcatggtctgcaccgtgagggccgtagcgcggatctgaagggccagctggcgcaacgtattgttgacgatgtgagcgttgcggcggaaatcgaccgtaaacacatttgggtgtacttcggcgagatgccggcgcagcaaatggttgaatatggccgtttcctgccgcagccgggtcatgagggtgaatggtttgacaacctgagcagcgatgaacgtgcgtttatggagaccaatgttgatgtgagccgtaccgagaacctgtattttcaaggcctcgagcaccaccaccaccaccac

SEQ ID NO: 55 - DNA, 1068 bp, synthetic construct
  misc_feature 1..1068: note = Engineered phosphite dehydrogenase (PTDH) from Pseudomonas stutzeri plus N-terminal His6-tag from pET-15b vector
  source 1..1068: mol_type = other DNA
atgggcagcagccatcatcatcatcatcacagcagcggcctggtgccgcgcggcagccatatgctgccgaaactcgttataactcaccgagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttccggggctggcaaccacggttctacggcacggggctggataacgctacggtcggcttccttggcatgggcgccatcggactggccatggctgatcgcttgcagggatggggcgcgaccctgcagtaccacgcggcgaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccaatcctgccgcagac

SEQ ID NO: 56 - DNA, 456 bp, synthetic construct
  misc_feature 1..456: note = Malonate semialdehyde decarboxylase (MSAD) from Coryneform sp. FG41 (NCBI: MZ369160) plus TEV protease recognition site and C-terminal His6-tag
  source 1..456: mol_type = other DNA
atgcccttaatccgtatagatcttaccagtgatcgttcgagagagcaacggcgggcgattgctgatgcagtccatgacgctttagtagaagttttagcgattccggctcgtgatcgcttccagatactgactgcgcacgatccctctgatattatagccgaagatgctggacttggctttcagcggtcccccagtgtagtcatcatacacgtctttacacaggcaggtagaactattgaaacgaaacagagagtatttgcagcgataacagaaagtctggctccaatcggtgttgcaggatctgatgtttttatcgcaatcaccgaaaatgcaccccatgactggagctttgggtttggcagtgcacaatatgtcacgggtgaacttgcgattccagccactggtgcggctgagaacctgtattttcaaggcctcgagcaccaccaccaccaccac

SEQ ID NO: 57 - DNA, 789 bp, synthetic construct
  misc_feature 1..789: note = YdfG from Escherichia coli plus TEV protease recognition site and C-terminal His6-tag
  source 1..789: mol_type = other DNA
atgatcgttttagtaactggagcaacggcaggttttggtgaatgcattactcgtcgttttattcaacaagggcataaagttatcgccactggccgtcgccaggaacggttgcaggagttaaaagacgaactgggagataatctgtatatcgcccaactggacgttcgcaaccgcgccgctattgaagagatgctggcatcgcttcctgccgagtggtgcaatattgatatcctggtaaataatgccggcctggcgttgggcatggagcctgcgcataaagccagcgttgaagactgggaaacgatgattgataccaacaacaaaggcctggtatatatgacgcgcgccgtcttaccgggtatggttgaacgtaatcatggtcatattattaacattggctcaacggcaggtagctggccgtatgccggtggtaacgtttacggtgcgacgaaagcgtttgttcgtcagtttagcctgaatctgcgtacggatctgcatggtacggcggtgcgcgtcaccgacatcgaaccgggtctggtgggtggtaccgagttttccaatgtccgctttaaaggcgatgacggtaaagcagaaaaaacctatcaaaataccgttgcattgacgccagaagatgtcagcgaagccgtctggtgggtgtcaacgctgcctgctcacgtcaatatcaataccctggaaatgatgccggttacccaaagctatgccggactgaatgtccaccgtcaggagaacctgtattttcaaggcctcgagcaccaccaccaccaccac

SEQ ID NO: 58 - DNA, 930 bp, synthetic construct
  misc_feature 1..930: note = MmsB from Pseudomonas putida KT2440 plus TEV protease recognition site and C-terminal His6-tag
  source 1..930: mol_type = other DNA
atgcgtatcgcattcatcggcctgggcaacatgggcgcgcccatggcccgcaacctgatcaaggccgggcatcagctgaacctgttcgacctgaacaaggccgtgctggccgagctggcagaactgggcgggcagatcagcccctcgcccaaggacgcggcggccaacagcgagctggtgatcaccatgctgccggccgcagcccatgtgcgtagcgtgtacttgaacgaggacggcgtactggccggtattcgtcctggcacgccgaccgttgactgcagcaccatcgacccgcagaccgcacgtgacgtgtccaaggccgcagcggcaaagggcgtggacatgggggatgcgccggtttccggtggtactggcggcgcggcggccggcaccctgacgttcatggtcggcgccagtaccgagttgttcgccagcctcaagccggtactggagcagatgggccgcaacatcgtgcactgcggggaagtcggtaccggccagatcgccaagatctgcaacaacctgctgctcggcatttcgatgatcggcgtgtccgaggccatggccctgggtaacgcgctgggtatcgataccaaggtgctggccggcatcatcaacagttcgaccgggcgttgctggagctcggacacctacaacccgtggccgggcatcatcgaaaccgcacctgcatcgcgtggctacaccggtggctttggcgccgaactcatgctcaaggacctggggttggccaccgaagcggcacgccaggcacaccaaccggtgattctcggtgccgtggcccagcagctgtaccaggccatgagcctgcgaggcgagggtggcaaggacttctcggccatcgtcgagggttatcgcaagaaggatgagaacctgtattttcaaggcctcgagcaccaccaccaccaccac

SEQ ID NO: 59 - protein, 163 aa, synthetic construct
  REGION 1..163: note = Cg10062 from Corynebacterium glutamicum codon-optimized for Escherichia coli plus TEV protease recognition site and C-terminal His6-tag
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVYITEIPGSNMTEYGRLLMEPGEEEKWFNSLPEGLRERLTELEGSSEENLYFQGLEHHHHHH

SEQ ID NO: 60 - protein, 163 aa, synthetic construct
  REGION 1..163: note = Cg10062(E114Q) from Corynebacterium glutamicum codon-optimized for Escherichia coli plus TEV protease recognition site and C-terminal His6-tag
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVYITEIPGSNMTQYGRLLMEPGEEEKWFNSLPEGLRERLTELEGSSEENLYFQGLEHHHHHH

SEQ ID NO: 61 - protein, 163 aa, synthetic construct
  REGION 1..163: note = Cg10062(E114D) from Corynebacterium glutamicum codon-optimized for Escherichia coli plus TEV protease recognition site and C-terminal His6-tag
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVYITEIPGSNMTDYGRLLMEPGEEEKWFNSLPEGLRERLTELEGSSEENLYFQGLEHHHHHH

SEQ ID NO: 62 - protein, 163 aa, synthetic construct
  REGION 1..163: note = Cg10062(E114N) from Corynebacterium glutamicum codon-optimized for Escherichia coli plus TEV protease recognition site and C-terminal His6-tag
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVYITEIPGSNMTNYGRLLMEPGEEEKWFNSLPEGLRERLTELEGSSEENLYFQGLEHHHHHH

SEQ ID NO: 63 - protein, 163 aa, synthetic construct
  REGION 1..163: note = Cg10062(H28A) from Corynebacterium glutamicum codon-optimized for Escherichia coli plus TEV protease recognition site and C-terminal His6-tag
PTYTCWSQRIRISREAKQRIAEAITDAAHELAHAPKYLVQVIFNEVEPDSYFIAAQSASENHIWVQATIRSGRTEKQKEELLLRLTQEIALILGIPNEEVWVYITEIPGSNMTEYGRLLMEPGEEEKWFNSLPEGLRERLTELEGSSEENLYFQGLEHHHHHH

SEQ ID NO: 64 - protein, 163 aa, synthetic construct
  REGION 1..163: note = Cg10062(R70A) from Corynebacterium glutamicum codon-optimized for Escherichia coli plus TEV protease recognition site and C-terminal His6-tag
PTYTCWSQRIRISREAKQRIAEAITDAHHELAHAPKYLVQVIFNEVEPDSYFIAAQSASENHIWVQATIASGRTEKQKEELLLRLTQEIALILGIPNEEVWVYITEIPGSNMTEYGRLLMEPGEEEKWFNSLPEGLRERLTELEGSSEENLYFQGLEHHHHHH

SEQ ID NO: 65 - protein, 163 aa, synthetic construct
  REGION 1..163: note = Cg10062(R70K) from Corynebacterium glutamicum codon-optimized for Escherichia coli plus TEV protease recognition site and C-terminal His6-tag
PTYTCWSQRIRISREAKQRIAEAITDAHHELAHAPKYLVQVIFNEVEPDSYFIAAQSASENHIWVQATIKSGRTEKQKEELLLRLTQEIALILGIPNEEVWVYITEIPGSNMTEYGRLLMEPGEEEKWFNSLPEGLRERLTELEGSSEENLYFQGLEHHHHHH

SEQ ID NO: 66 - protein, 163 aa, synthetic construct
  REGION 1..163: note = Cg10062(R73A) from Corynebacterium glutamicum codon-optimized for Escherichia coli plus TEV protease recognition site and C-terminal His6-tag
PTYTCWSQRIRISREAKQRIAEAITDAHHELAHAPKYLVQVIFNEVEPDSYFIAAQSASENHIWVQATIRSGATEKQKEELLLRLTQEIALILGIPNEEVWVYITEIPGSNMTEYGRLLMEPGEEEKWFNSLPEGLRERLTELEGSSEENLYFQGLEHHHHHH

SEQ ID NO: 67 - protein, 163 aa, synthetic construct
  REGION 1..163: note = Cg10062(R73K) from Corynebacterium glutamicum codon-optimized for Escherichia coli plus TEV protease recognition site and C-terminal His6-tag
PTYTCWSQRIRISREAKQRIAEAITDAHHELAHAPKYLVQVIFNEVEPDSYFIAAQSASENHIWVQATIRSGKTEKQKEELLLRLTQEIALILGIPNEEVWVYITEIPGSNMTEYGRLLMEPGEEEKWFNSLPEGLRERLTELEGSSEENLYFQGLEHHHHHH

SEQ ID NO: 68 - protein, 163 aa, synthetic construct
  REGION 1..163: note = Cg10062(Y103A) from Corynebacterium glutamicum codon-optimized for Escherichia coli plus TEV protease recognition site and C-terminal His6-tag
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVAITEIPGSNMTEYGRLLMEPGEEEKWFNSLPEGLRERLTELEGSSEENLYFQGLEHHHHHH

SEQ ID NO: 69 - protein, 163 aa, synthetic construct
  REGION 1..163: note = Cg10062(Y103F) from Corynebacterium glutamicum codon-optimized for Escherichia coli plus TEV protease recognition site and C-terminal His6-tag
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVFITEIPGSNMTEYGRLLMEPGEEEKWFNSLPEGLRERLTELEGSSEENLYFQGLEHHHHHH

SEQ ID NO: 70 - protein, 163 aa, synthetic construct
  REGION 1..163: note = Cg10062(E114A) from Corynebacterium glutamicum codon-optimized for Escherichia coli plus TEV protease recognition site and C-terminal His6-tag
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVYITEIPGSNMTAYGRLLMEPGEEEKWFNSLPEGLRERLTELEGSSEENLYFQGLEHHHHHH

SEQ ID NO: 71 - protein, 163 aa, synthetic construct
  REGION 1..163: note = Cg10062(E114D-Y103F) from Corynebacterium glutamicum codon-optimized for Escherichia coli plus TEV protease recognition site and C-terminal His6-tag
PTYTCWSQRIRISREAKQRIAEAITDAHHELAHAPKYLVQVIFNEVEPDSYFIAAQSASENHIWVQATIRSGRTEKQKEELLLRLTQEIALILGIPNEEVWVFITEIPGSNMTDYGRLLMEPGEEEKWFNSLPEGLRERLTELEGSSEENLYFQGLEHHHHHH

SEQ ID NO: 72 - protein, 164 aa, synthetic construct
  REGION 1..164: note = Cis-CaaD from Coryneform sp. codon-optimized for Escherichia coli plus TEV protease recognition site and C-terminal His6-tag
PVYMVYVSQDRLTPSAKHAVAKAITDAHRGLTGTQHFLAQVNFQEQPAGNVFLGGVQQGGDTIFVHGLHREGRSADLKGQLAQRIVDDVSVAAEIDRKHIWVYFGEMPAQQMVEYGRFLPQPGHEGEWFDNLSSDERAFMETNVDVSRTENLYFQGLEHHHHHH

SEQ ID NO: 73 - protein, 356 aa, synthetic construct
  REGION 1..356: note = Engineered phosphite dehydrogenase (PTDH) from Pseudomonas stutzeri plus N-terminal His6-tag from pET-15b vector
MGSSHHHHHHSSGLVPRGSHMLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGAIGLAMADRLQGWGATLQYHAAKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKANPAAD

SEQ ID NO: 74 - protein, 151 aa, synthetic construct
  REGION 1..151: note = Malonate semialdehyde decarboxylase (MSAD) from Coryneform sp. FG41 plus TEV protease recognition site and C-terminal His6-tag
PLIRIDLTSDRSREQRRAIADAVHDALVEVLAIPARDRFQILTAHDPSDIIAEDAGLGFQRSPSVVIIHVFTQAGRTIETKQRVFAAITESLAPIGVAGSDVFIAITENAPHDWSFGFGSAQYVTGELAIPATGAAENLYFQGLEHHHHHH

SEQ ID NO: 75 - protein, 263 aa, synthetic construct
  REGION 1..263: note = YdfG from Escherichia coli plus TEV protease recognition site and C-terminal His6-tag
MIVLVTGATAGFGECITRRFIQQGHKVIATGRRQERLQELKDELGDNLYIAQLDVRNRAAIEEMLASLPAEWCNIDILVNNAGLALGMEPAHKASVEDWETMIDTNNKGLVYMTRAVLPGMVERNHGHIINIGSTAGSWPYAGGNVYGATKAFVRQFSLNLRTDLHGTAVRVTDIEPGLVGGTEFSNVRFKGDDGKAEKTYQNTVALTPEDVSEAVWWVSTLPAHVNINTLEMMPVTQSYAGLNVHRQENLYFQGLEHHHHHH

SEQ ID NO: 76 - protein, 310 aa, synthetic construct
  REGION 1..310: note = MmsB from Pseudomonas putida KT2440 plus TEV protease recognition site and C-terminal His6-tag
MRIAFIGLGNMGAPMARNLIKAGHQLNLFDLNKAVLAELAELGGQISPSPKDAAANSELVITMLPAAAHVRSVYLNEDGVLAGIRPGTPTVDCSTIDPQTARDVSKAAAAKGVDMGDAPVSGGTGGAAAGTLTFMVGASTELFASLKPVLEQMGRNIVHCGEVGTGQIAKICNNLLLGISMIGVSEAMALGNALGIDTKVLAGIINSSTGRCWSSDTYNPWPGIIETAPASRGYTGGFGAELMLKDLGLATEAARQAHQPVILGAVAQQLYQAMSLRGEGGKDFSAIVEGYRKKDENLYFQGLEHHHHHH

SEQ ID NO: 77 - DNA, 23 bp, synthetic construct
  misc_feature 1..23: note = AS001 (F) Primer
  source 1..23: mol_type = other DNA
caacatgacccagtatggccgtc

SEQ ID NO: 78 - DNA, 18 bp, synthetic construct
  misc_feature 1..18: note = AS002 (R) Primer
  source 1..18: mol_type = other DNA
ctacccgggatttcggta

SEQ ID NO: 79 - DNA, 23 bp, synthetic construct
  misc_feature 1..23: note = AS003 (F) Primer
  source 1..23: mol_type = other DNA
caacatgaccgattatggccgtc

SEQ ID NO: 80 - DNA, 29 bp, synthetic construct
  misc_feature 1..29: note = AS012 (F) Primer
  source 1..29: mol_type = other DNA
taccgacgcggcgcatgaactggcgcacg

SEQ ID NO: 81 - DNA, 18 bp, synthetic construct
  misc_feature 1..18: note = AS013 (R) Primer
  source 1..18: mol_type = other DNA
atcgcttccgcgatgcgt

SEQ ID NO: 82 - DNA, 24 bp, synthetic construct
  misc_feature 1..24: note = AS014 (F) Primer
  source 1..24: mol_type = other DNA
agcgaccatcgcgagcggccgtac

SEQ ID NO: 83 - DNA, 18 bp, synthetic construct
  misc_feature 1..18: note = AS015 (R) Primer
  source 1..18: mol_type = other DNA
tgaacccaaatgtggttc

SEQ ID NO: 84 - DNA, 23 bp, synthetic construct
  misc_feature 1..23: note = AS016 (F) Primer
  source 1..23: mol_type = other DNA
agcgaccatcaaaagcggccgta

SEQ ID NO: 85 - DNA, 20 bp, synthetic construct
  misc_feature 1..20: note = AS017 (R) Primer
  source 1..20: mol_type = other DNA
tgaacccaaatgtggttctc

SEQ ID NO: 86 - DNA, 23 bp, synthetic construct
  misc_feature 1..23: note = AS018 (F) Primer
  source 1..23: mol_type = other DNA
ccgtagcggcgcgaccgaaaagc

SEQ ID NO: 87 - DNA, 18 bp, synthetic construct
  misc_feature 1..18: note = AS019 (R) Primer
  source 1..18: mol_type = other DNA
atggtcgcttgaacccaa

SEQ ID NO: 88 - DNA, 23 bp, synthetic construct
  misc_feature 1..23: note = AS020 (F) Primer
  source 1..23: mol_type = other DNA
ccgtagcggcaaaaccgaaaagc

SEQ ID NO: 89 - DNA, 30 bp, synthetic construct
  misc_feature 1..30: note = AS021 (F) Primer
  source 1..30: mol_type = other DNA
agtgtgggttgcgattaccgaaatcccggg

SEQ ID NO: 90 - DNA, 18 bp, synthetic construct
  misc_feature 1..18: note = AS022 (R) Primer
  source 1..18: mol_type = other DNA
tcctcgttcgggataccc

SEQ ID NO: 91 - DNA, 27 bp, synthetic construct
  misc_feature 1..27: note = AS023 (F) Primer
  source 1..27: mol_type = other DNA
agtgtgggtttttattaccgaaatccc

SEQ ID NO: 92 - DNA, 25 bp, synthetic construct
  misc_feature 1..25: note = AS024 (F) Primer
  source 1..25: mol_type = other DNA
caacatgaccgcgtatggccgtctg

SEQ ID NO: 93 - DNA, 25 bp, synthetic construct
  misc_feature 1..25: note = AS026 (F) Primer
  source 1..25: mol_type = other DNA
caacatgaccaactatggccgtctg